(19) United States
(12) Patent Application Publication
(10) Pub. No.: US 2018/0186823 A1
(43) Pub. Date: Jul. 5, 2018

(54) PLATINUM COMPOUNDS, COMPOSITIONS, AND USES THEREOF

(71) Applicant: PLACON THERAPEUTICS, INC., Watertown, MA (US)

(72) Inventors: Sudhakar Kadiyala, Newton, MA (US); Benoît Moreau, Newton, MA (US); Mark T. Bilodeau, Concord, MA (US); Kerry Whalen, Waltham, MA (US); Sukhjeet Singh, Lexington (CA); Richard Wooster, Natick, MA (US); Charles-Andre Lemelin, North Chelmsford, MA (US)

(21) Appl. No.: 15/737,073

(22) PCT Filed: Jun. 22, 2016
(86) PCT No.: PCT/US2016/038728

§ 371 Date: Dec. 15, 2017

Related U.S. Application Data
(60) Provisional application No. 62/183,434, filed on Jun. 23, 2015.

(51) Int. Cl. C07F 15/00; A61P 35/00

(52) U.S. Cl. CPC C07F 15/0093; A61P 35/00

(57) ABSTRACT

The present teachings relate to compounds and compositions comprising a platinum (IV) complex having at least one reacting group for reacting with a functional group on a protein, engineered protein, antibody, antibody fragment, peptide, agonist, antagonist, aptamer or any moiety which may be capable of recognizing a selected target cell population, and/or derivatives/analogs/mimics thereof. The platinum (IV) complex may comprise at least one active agent.

### REFERENCED TO RELATED APPLICATIONS

The present application claims priority to U.S. Provisional Patent Application No. 62/183,434, filed Jun. 23, 2015, entitled PLATINUM CompoundS, COMPOSITIONS, AND USES THEREOF, the contents of which are herein incorporated by reference in their entirety.

### FIELD OF THE INVENTION

The invention relates to platinum based compounds.

### BACKGROUND OF THE INVENTION

Platinum-based drugs are among the most active and widely used anticancer agents. Cisplatin is one of the few FDA-approved, platinum-based cancer chemotherapeutics. Although cisplatin is effective against a number of solid tumors, especially testicular and ovarian cancer, its clinical use has been limited because of its toxic effects as well as the intrinsic and acquired resistance of some tumors to this drug.

To overcome these limitations, platinum analogs with lower toxicity and greater activity in cisplatin-resistant tumors have been developed and tested, resulting in the approval of carboplatin and oxaliplatin in the United States. For example, carboplatin has the advantage of being less nephrotoxic, but its cross-resistance with cisplatin has limited its application in otherwise cisplatin-treatable diseases.

Oxaliplatin, however, exhibits a different anticancer spectrum from that of cisplatin. It has been approved as the first or second line therapy in combination with 5-fluorouracil/leucovorin for advanced colorectal cancer, for which cisplatin and carboplatin are essentially inactive. These platinum drugs have platinum in the 2+ oxidative state (Pt(II)) and are not orally active.

Platinum complexes in the 4+ oxidative state (Pt(IV) complexes) provide several advantages. Platinum(IV) complexes are substantially inactive in the 4+ oxidation state but become activated upon reduction to the platinum(II) state. As such Pt(IV) complexes constitute prodrugs of Pt(II) drugs that are activated in tumor cells. The two additional coordination sites (the axial sites) can also be modified to change the pharmacokinetic properties of the complexes. For example, the two axial sites, as well as the four equatorial sites, can include ligands that have a Michael acceptor. The inclusion of a Michael acceptor as disclosed in the present teachings may increase the Pt concentration in tumor cells and, in certain instances, may increase the efficacy in treating a disease or a condition discussed herein. In certain instances, Pt(IV) complexes of the present teachings can be orally active and/or have a reduced long-term toxicity.

### SUMMARY OF THE INVENTION

The present teachings relate to compositions, for example, for reducing, disrupting, or inhibiting the growth of a cancer cell or inducing the death of a cancer cell.

The composition can include a platinum (IV) compound. In various embodiments, the platinum (IV) compound includes a suitable reacting group for reacting with a functional group on a protein, engineered protein, antibody, antibody fragment, peptide, agonist, antagonist, aptamer or any moiety which may be capable of recognizing a selected target cell population, and/or derivatives/analogs/mimics thereof. Such compounds are referred to herein as Pt(IV)M. The reacting group may be a Michael acceptor. For example, a Michael acceptor can be introduced by a linker between platinum and the Michael acceptor and/or alkylating functionality. In various embodiments, one of or both the axial ligands each comprises one or more Michael acceptors and/or alkylating functionality. In some embodiments, the protein is albumin and the reacting group binds to albumin.

The Pt(IV)M compound has a general structure of:

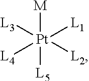

wherein M is a ligand comprising any suitable reacting group for reacting with a functional group on a protein; L1, L2, L3, L4, L5 may be any suitable ligand for Pt(IV). M may further comprise a linker between the reacting group and platinum. M may be at the any of the two axial sites, as well as the four equatorial sites of platinum.

In another aspect, the present application provides a Pt(IV)M compound, wherein the compound comprises at least one active agent as a ligand of Pt(IV). The active agent may be convalently connected to Pt(IV) with any suitable linker. The linker may be a cleavable linker that can be cleaved hydolytically, reductively, enzymatically, or in a pH-dependent manner. The Pt(IV)M compound comprising at least one active agent has a general structure of:

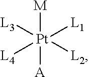

wherein M is a ligand comprising any suitable group for reacting with a function group on a protein; A is a ligand comprising an active agent; L1, L2, L3, and L4 may be any suitable ligand for Pt(IV). M may further comprise a linker between the reacting group and platinum. M may be at the any of the two axial sites, as well as the four equatorial sites of platinum. A may further comprise a linker between the active agent and platinum. A may be at the any of the two axial sites, as well as the four equatorial sites of platinum.

In another aspect, the present application provides a combination of a Pt(IV)M compound and at least one other active agent. The at least one other active agent may be a cytostatic agent. The Pt(IV)M compound and the at least one other active agent may be administered simultaneously or sequentially.

The present teachings also provide compositions including a Pt(IV)M compound as described herein and methods of using a Pt(IV)M compound or a composition as described herein. In various embodiments, the methods of the present teachings are useful for the prevention or treatment of diseases that benefit from increased cell death or decreased cell proliferation. For example, the method of the present teachings can be used to increase cancer cell death or decrease cancer cell proliferation. The increased cancer cell death or decreased cancer proliferation can occur, for example, outside the body (in vitro) or inside the body (in vivo).

Certain embodiments of the present teachings also provide for use of a compound as described herein as a medicament for treating or preventing a disease and/or in the manufacture of such a medicament, e.g., for use in the treatment of a disease. Some embodiments provide the use of a Pt(IV)M compound as described herein for use as a medicament. In certain embodiments, the teachings provide a compound or composition as described herein for the treatment of disease, e.g. for the treatment of a cancer. In certain embodiments, the teachings provide a compound or composition as described herein for the treatment of a tumor, wherein the tumor cells express one or more KRAS mutantations. Some embodiments provide the use of a Pt(IV)M compound as described herein in combination with at least one other active agent or another therapy.

### BRIEF DESCRIPTION OF DRAWINGS

FIG. 1 is a graph illustrating growth curves of A2780 tumors in nude mouse xenografts when mice were dosed with two control drugs, vehicle or three Pt(IV)M of the present teachings.

FIG. 2 is a graph illustrating growth curves of A2780 tumors in nude mouse xenografts when the mice were dosed with two control drugs, vehicle or three Pt(IV)M of the present teachings.

FIG. 3 is a graph illustrating growth curves of A2780 tumors in nude mouse xenografts when the mice were dosed with two control drugs, vehicle or two Pt(IV)M of the present teachings.

FIG. 4 is a graph illustrating growth curves of A2780 tumors in nude mouse xenografts when the mice were dosed with two control drugs, vehicle or a Pt(IV)M of the present teachings.

FIG. 5 is a graph illustrating growth curves of Calu-6 tumors in nude mouse xenografts when the mice were dosed with two control drugs, vehicle or a Pt(IV)M of the present teachings.

FIG. 6 is a graph illustrating growth curves of A2780 tumors in nude mouse xenografts when the mice were dosed with two control drugs, vehicle or two Pt(IV)M of the present teachings.

FIG. 7 is a graph illustrating growth curves of Calu-6 tumors in nude mouse xenografts when the mice were dosed with two control drugs, vehicle or two Pt(IV)M of the present teachings.

FIG. 8 is a graph depicting platinum levels in tumor when platinum (IV) was dosed in the form of eight exemplary compounds of the present teachings and two comparison compounds to tumor-bearing nude mice via intravenous administration.

FIG. 9 is an liquid chromatography-inductively coupled plasma mass spectrometry (LC-ICPMS) chromatogram showing the retention time of a Pt(IV)M of the present teachings.

FIG. 10 is an LC-ICPMS chromatogram showing the retention time of a product of the incubation of a Pt(IV)M of the present teachings with commercial albumin.

FIG. 11 is an LC-ICPMS chromatogram showing the retention time of a product of the incubation of a Pt(IV)M of the present teachings with rat serum.

FIG. 12 is a graph illustrating growth curves of KRAS mutant Calu-6 tumors when the mice were dosed with a control drug, vehicle or a Pt(IV)M of the present teachings.

FIG. 13 is a graph illustrating albumin uptake in KRAS mutant cells and KRAS WT cells in vitro.

FIG. 14 shows uptake of fluorescently labeled albumin in KRAS mutant cells and KRAS WT cells in vitro.

FIG. 15 is a graph illustrating growth curves of KRAS wild type BxPC-3 pancreatic cancer model when the mice were dosed with a control drug, vehicle or a Pt(IV)M of the present teachings.

FIG. 16 is a graph illustrating growth curves of KRAS mutant Miapaca-2 pancreatic cancer model when the mice were dosed with a control drug, vehicle or a Pt(IV)M of the present teachings.

FIG. 17 shows TGI % of cisplatin, oxliplatin, bismaleimide compounds and Pt(IV)M monomaleimide compounds.

FIG. 18 shows platinum accumulation in plasma and tumor with a single dose of a Pt(IV)M monomaleimide compound and cisplatin.

FIG. 19 shows platinum accumulation and DNA platination in plasma and tumor with two doses of a Pt(IV)M monomaleimide compound and cisplatin.

FIG. 20 compares tumor volume in ovarian cancer model A2780 with treatment with cisplatin and a Pt(IV)M monomaleimide compound.

FIG. 21 shows post-studt platinum levels in ovarian cancer model A2780 with treatment with cisplatin and a Pt(IV)M monomaleimide compound.

FIG. 22 compares tumor volume after multiple doses of cisplatin and a Pt(IV)M monomaleimide compound in lung cancer model NCI-H520 for 32 days.

FIG. 23 shows platinum levels after multiple doses of cisplatin and a Pt(IV)M monomaleimide compound in lung cancer model NCI-H520 for 32 days.

FIG. 24 shows cell dedifferentiation images from day 10 of NCI-H520 study.

FIG. 25 shows TUNEL apoptosis images from day 10 of NCI-H520 study.

FIG. 26 shows platinum concentrations in rats over a period of up to 100 hours after treatment with cisplatin and a Pt(IV)M monomaleimide compound.

FIG. 27 shows platinum concentrations in dogs over a period of up to 400 hours after treatment with cisplatin and a Pt(IV)M monomaleimide compound.

FIG. 28 shows RBC partitioning and protein partitioning after treatment with a Pt(IV)M monomaleimide compound.

FIG. 29 shows average tumor volumes and platinum levels in the tumors in MX-1 breast cancer model treated with cisplatin and a Pt(IV)M monomaleimide compound.

FIG. 30 shows levels of blood markers of kidney damage after treatments with cisplatin and a Pt(IV)M monomaleimide compound.

### DETAILED DESCRIPTION

Applicants have discovered that Pt(IV) compounds having a suitable reacting group for reacting with a functional group on a protein, engineered protein, antibody, antibody fragment, peptide, agonist, antagonist, aptamer or any moiety which may be capable of recognizing a selected target cell population, and/or derivatives/analogs/mimics thereof, are effective inhibitors of cellular proliferation and tumor growth. Such compounds are referred to herein as Pt(IV)M compounds.

The term “reacting group” as used herein refers to a functional group of the Pt(IV) compounds that may react with a functional group on a protein, engineered protein, antibody, antibody fragment, peptide, agonist, antagonist, aptamer or any moiety which may be capable of recognizing a selected target cell population, and/or derivatives/analogs/mimics thereof. The functional group on a protein, engineered protein, antibody, antibody fragment, peptide, agonist, antagonist, aptamer or any moiety which may be capable of recognizing a selected target cell population, and/or derivatives/analogs/mimics thereof, may be amino groups, hydroxyl groups or thiol groups.

Non-limiting examples of a reacting group include an activated disulfide group, a vinylcarbonyl group, a vinyl acetylene group, an epoxide, an aziridine group or an acetylene group. The groups may be substituted, where appropriate. The reacting group may also be any of:

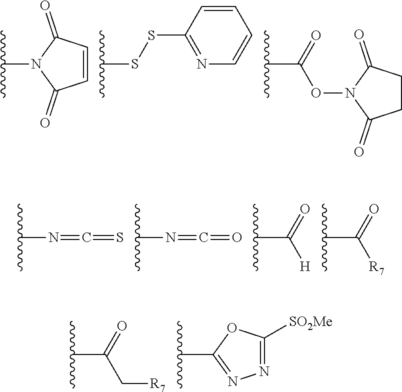

where R⁷ is Cl, Br, F, mesylate, tosylate, O-(4-nitrophenyl), O-pentafluorophenyl.

The protein, engineered protein, antibody, antibody fragment, peptide, agonist, antagonist, aptamer or any moiety which may be capable of recognizing a selected target cell population, and/or derivatives/analogs/mimics thereof may be any moiety disclosed in EP 0554708 to Willner et al. (BMS), the contents of which are incorporated herein by reference in their entirety. For example, the protein, engineered protein, antibody, antibody fragment, peptide, agonist, antagonist, aptamer or any moiety which may be capable of recognizing a selected target cell population, and/or derivatives/analogs/mimics thereof may be non-immunoreactive, such as but not limited to transferrin, epidermal growth factors (“EGF”), bombesin, gastrin, gastrin-releasing peptide, platelet-derived growth factor, IL-2, IL-6, tumor growth factors (“TGF”), such as TGF-α and TGF-β, vaccinia growth factor (“VGF”), insulin and insulin-like growth factors I and II. Non-peptidyl moieties may include, for example, steroids, carbohydrates and lectins. The protein, engineered protein, antibody, antibody fragment, peptide, agonist, antagonist, aptamer or any moiety which may be capable of recognizing a selected target cell population, and/or derivatives/analogs/mimics thereof may also be non-immunoreactive such as but not limited to an antigen-recognizing immunoglobulin (also referred to as “antibody”), or antigen-recognizing fragment thereof. The immunoglobulins may be immunoglobulins which can recognize a tumor-associated antigen. As used, “immunoglobulin” may refer to any recognized class or subclass of immunoglobulins such as IgG, IgA, IgM, IgD, or IgE. The immunoglobulin can be derived from any species such as human, murine, or rabbit origin. Further, the immunoglobulin may be polyclonal, monoclonal, chimeric, bifunctional or hybrid.

In some embodiments, the protein is albumin or derivatives/analogs/mimics thereof. In some embodiments, the engineered protein may be a recombinant albumin (rAlbumin) such as the recombinant albumin disclosed in US 20090280534 to Christensen et al. (Novozymes), the contents of which are incorporated herein by reference in their entirety.

The reacting group may be a Michael acceptor and/or alkylating functionality. In some embodiments, the Pt(IV)M compounds comprise a maleimide group and/or derivatives thereof.

“Michael acceptor”, as used herein, refers to an α,β-unsaturated electrophile, such as, but not limited to, an α,β-un saturated carbonyl derivative or an α,β-unsaturated nitrile: “Electrophile” means able to accept an electron pair; “α,β-unsaturated electrophile” means the compound class that includes, but is not limited to, α,β-unsaturated carbonyl derivative, α,β-unsaturated nitrile, α,β-unsaturated sulfone, or other vinyl derivative substituted with a strong electron withdrawing group, such as, but not limited to, a nitro group; “α,β-unsaturated carbonyl derivative” means the compound class that includes, but is not limited to, -unsaturated ketone, quinone or derivative thereof, α,β-unsaturated aldehyde, α,β-unsaturated carboxylic acid derivative, such as, but not limited to, an ester, an amide, a substituted amide, or a maleimide or a derivative thereof.

Toxicity may further be measured relative to a subject's weight loss where weight loss over 15%, over 20% or over 30% of the body weight is indicative of toxicity. Other metrics of toxicity may also be measured such as patient presentation metrics including lethargy and general malaiase. Neutropenia or thrombopenia may also be metrics of toxicity.

Pharmacologic indicators of toxicity include elevated AST/ALT levels, neurotoxicity, kidney damage, GI damage and the like.

Furthermore, in some embodiments, such compounds are effective for inhibiting tumor growth, whether measured as a net value of size (weight, surface area or volume) or as a rate over time, in multiple types of tumors.

In some embodiments the size of a tumor is reduced by 60% or more. In some embodiments, the size of a tumor is reduced by at least 20%, at least 30%, at least 40%, at least 50%, at least 60%, at least 70%, at least 80%, at least 90%, at least 95%, at least 96%, at least 97%, at least 98%, at least 99%, at least 100%, by a measure of weight, and/or area and/or volume.

In some embodiments, the RECIST (Response Evaluation Criteria In Solid Tumors) criteria are used to characterize the effects of the compounds of the invention on solid tumors. The guidelines for gauging tumors were updated and published in the European Journal of Cancer (EJC) in January 2009 (Eisenhauer, et al., European Journal of Cancer: 45 (2009) 228-247), the contents of which are incorporated herein by reference in their entirety. Any of the RECIST metrics may be used to characterize the effects of the compounds of the invention on tumors including but not limited to response, assessment and measurement criteria.

It has been surprisingly found that the relative ability of compounds of the invention to inhibit in vitro cell proliferation is not predictive of their relative ability to inhibit tumor growth, i.e., their relative ability to inhibit tumor growth is greater than their relative ability to inhibit cell proliferation in vitro.

Without wishing to be bound to any theory, the effective delivery of a Pt(IV)M compound may be related to the covalent attachment of the compound to a protein such as albumin. Conjugation to albumin prevents rapid clearance and delivers stable and inactive form of platinum to tumor sites. The compound-albumin bond may be cleaved at a tumor site, creating an active platinum compound, e.g., a Pt(II) compound. Trafficking of a Pt(IV)M compound by albumin is being studied with MIA PaCa-2 and BxPC-3 cell lines (Commisso et al., Nature, vol. 497:633-637 (2013), the contents of which are incorporated herein by reference in their entirety).

In some embodiments, a Pt(IV)M compound as described herein is administered to a subject who has a tumor comprising cells that express one or more KRAS mutations. A subject's tumor may be assayed for KRAS mutations using methods known in the art, for example, see Anderson, 2011, Expert Rev Mol Diagn. 11:635-642 and Thierry et al., 2014, Nature Medicine 20:430-435, the contents of each of which are incorporated herein by reference in their entirety. If the tumor has a KRAS mutation, the tumor is likely to be responsive to treatment by the Pt(IV)M compounds disclosed herein. In some embodiments, the tumor is directly assayed for the presence of a KRAS mutation. In some embodiments, a non-tumor tissue, e.g., plasma DNA is assayed for the presence of a KRAS mutation.

This finding is also important because some tumors containing cells that express one or more KRAS mutants are not sensitive to certain treatments. For example, colorectal cancer patients are tested for the presence of KRAS mutations because the presence of certain of these mutations predicts resistance to therapies directed against EGFR (Siena et al., 2009, J Natl Cancer Inst 101:1308-24, the contents of which are incorporated herein by reference in their entirety). Such patients are candidates for treatments with a Pt(IV)M compound described herein.

For convenience, before further description of the present teachings, certain definitions of terms employed in the specification and claims are collected here. These definitions should be read in light of the remainder of the disclosure and as understood by a person of ordinary skill in the art. Unless defined otherwise, all technical and scientific terms used herein have the same meaning as commonly understood by a person of ordinary skill in the art.

The articles “a” and “an,” as used herein, should be understood to mean “at least one,” unless clearly indicated to the contrary.

The phrase “and/or,” as used herein, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements).

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements.

In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein, the phrase “at least one” in reference to a list of one or more elements should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, all transitional phrases such as “comprising,” “including,” “carrying,” “having,” “containing,” “involving,” “holding,” and the like are to be understood to be open-ended, i.e., to mean including but not limited to.

Only the transitional phrases “consisting of” and “consisting essentially of” shall be closed or semi-closed transitional phrases, respectively, as set forth in the United States Patent Office Manual of Patent Examining Procedures.

As used herein, a “subject” or a “patient” refers to any mammal (e.g., a human), such as a mammal that may be susceptible to a disease or disorder, for example, tumorigenesis or cancer. Examples include a human, a non-human primate, a cow, a horse, a pig, a sheep, a goat, a dog, a cat, or a rodent such as a mouse, a rat, a hamster, or a guinea pig. In various embodiments, a subject refers to one that has been or will be the object of treatment, observation, or experiment. For example, a subject can be a subject diagnosed with cancer or otherwise known to have cancer or one selected for treatment, observation, or experiment on the basis of a known cancer in the subject.

As used herein, “treatment” or “treating” refers to amelioration of a disease or disorder, or at least one sign or symptom thereof. “Treatment” or “treating” can refer to reducing the progression of a disease or disorder, as determined by, e.g., stabilization of at least one sign or symptom or a reduction in the rate of progression as determined by a reduction in the rate of progression of at least one sign or symptom. In another embodiment, “treatment” or “treating” refers to delaying the onset of a disease or disorder.

As used herein, “prevention” or “preventing” refers to a reduction of the risk of acquiring or having a sign or symptom a given disease or disorder, i.e., prophylactic treatment.

The phrase “therapeutically effective amount” as used herein means that amount of a compound, material, or composition comprising a compound of the present teachings that is effective for producing a desired therapeutic effect. Accordingly, a therapeutically effective amount treats or prevents a disease or a disorder, e.g., ameliorates at least one sign or symptom of the disorder. In various embodiments, the disease or disorder is a cancer.

A dash (“—”) that is not between two letters or symbols is used to indicate a point of attachment for a substituent. For example, —CONH₂ is attached through the carbon atom (C).

By “optional” or “optionally,” it is meant that the subsequently described event or circumstance may or may not occur, and that the description includes instances where the event or circumstance occurs and instances in which it does not. For example, “optionally substituted aryl” encompasses both “aryl” and “substituted aryl” as defined herein. It will be understood by those ordinarily skilled in the art, with respect to any group containing one or more substituents, that such groups are not intended to introduce any substitution or substitution patterns that are sterically impractical, synthetically non-feasible, and/or inherently unstable.

The term “alkyl” as used herein refers to a saturated straight or branched hydrocarbon, such as a straight or branched group of 1-22, 1-8, 1-6, or 1-4 carbon atoms, referred to herein as (C₁-C₂₂)alkyl, (C₁-C₈)alkyl, (C₁-C₆)alkyl, and (C₁-C₄)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “alkenyl” as used herein refers to an unsaturated straight or branched hydrocarbon having at least one carbon-carbon double bond (shown, for example, as “=”), such as a straight or branched group of 2-22, 2-8, 2-6, or 2-4 carbon atoms, referred to herein as (C₂-C₂₂)alkenyl, (C₂-C₈)alkenyl, (C₂-C₆)alkenyl, and (C₂-C₄)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkynyl” as used herein refers to an unsaturated straight or branched hydrocarbon having at least one carbon-carbon triple bond (shown, for example, as “≡”), such as a straight or branched group of 2-22, 2-8, 2-6, 2-4 carbon atoms, referred to herein as (C₂-C₂₂)alkynyl, (C₂-C₈)alkynyl, (C₂-C₆)alkynyl, and (C₂-C₄)alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “cycloalkyl” as used herein refers to a saturated or unsaturated monocyclic, bicyclic, other multicyclic, or bridged cyclic hydrocarbon group. A cyclocalkyl group can have 3-22, 3-12, or 3-8 ring carbons, referred to herein as (C₃-C₂₂)cycloalkyl, (C₃-C₁₂)cycloalkyl, or (C₃-C₈)cycloalkyl, respectively. A cycloalkyl group can also have one or more carbon-carbon double bond or carbon-carbon triple bond.

Exemplary monocyclic cycloalkyl groups include, but are not limited to, cyclopentanes (cyclopentyls), cyclopentenes (cyclopentenyls), cyclohexanes (cyclohexyls), cyclohexenes (cyclopexenyls), cycloheptanes (cycloheptyls), cycloheptenes (cycloheptenyls), cyclooctanes (cyclooctyls), cyclooctenes (cyclooctenyls), cyclononanes (cyclononyls), cyclononenes (cyclononenyls), cyclodecanes (cyclodecyls), cyclodecenes (cyclodecenyls), cycloundecanes (cycloundecyls), cycloundecenes (cycloundecenyls), cyclododecanes (cyclododecyls), and cyclododecenes (cyclododecenyls). Other exemplary cycloalkyl groups, including bicyclic, multicyclic, and bridged cyclic groups, include, but are not limited to, bicyclobutanes (bicyclobutyls), bicyclopentanes (bicyclopentyls), bicyclohexanes (bicyclohexyls), bicycleheptanes (bicycloheptyls, including bicyclo[2,2,1]heptanes (bicycle[2,2,1]heptyls) and bicycle[3,2,0]heptanes (bicycle[3,2,0]heptyls)), bicyclooctanes (bicyclooctyls, including octahydropentalene (octahydropentalenyl), bicycle[3,2,1]octane (bicycle[3,2,1]octyl), and bicylo[2,2,2]octane (bicycle[2,2,2]octyl)), and adamantanes (adamantyls). Cycloalkyl groups can be fused to other cycloalkyl saturated or unsaturated, aryl, or heterocyclyl groups.

The term “aryl” as used herein refers to a mono-, bi-, or other multi-carbocyclic aromatic ring system. The aryl can have 6-22, 6-18, 6-14, or 6-10 carbons, referred to herein as (C₆-C₂₂)aryl, (C₆-C₁₈)aryl, (C₆-C₁₄)aryl, or (C₆-C₁₀)aryl, respectively. The aryl group can optionally be fused to one or more rings selected from aryls, cycloalkyls, and heterocyclyls. The term “bicyclic aryl” as used herein refers to an aryl group fused to another aromatic or non-aromatic carbocylic or heterocyclic ring. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C₆)aryl” or phenyl. The phenyl group can also be fused to a cyclohexane or cyclopentane ring to form another aryl.

The term “arylalkyl” as used herein refers to an alkyl group having at least one aryl substituent (e.g., -aryl-alkyl-). Exemplary arylalkyl groups include, but are not limited to, arylalkyls having a monocyclic aromatic ring system, wherein the ring comprises 6 carbon atoms, referred to herein as “(C₆)arylalkyl.” The term “benzyl” as used herein refers to the group —CH₂-phenyl.

The term “heteroalkyl” refers to an alkyl group as described herein in which one or more carbon atoms is replaced by a heteroatom. Suitable heteroatoms include oxygen, sulfur, nitrogen, phosphorus, and the like. Examples of heteroalkyl groups include, but are not limited to, alkoxy, amino, thioester, and the like.

The terms “heteroalkenyl” and “heteroalkynyl” refer to unsaturated aliphatic groups analogous in length and possible substitution to the heteroalkyls described above, but that contain at least one double or triple bond, respectively.

The term “heterocycle” refers to cyclic groups containing at least one heteroatom as a ring atom, in some cases, 1 to 3 heteroatoms as ring atoms, with the remainder of the ring atoms being carbon atoms. Suitable heteroatoms include oxygen, sulfur, nitrogen, phosphorus, and the like. In some cases, the heterocycle may be 3- to 10-membered ring structures or 3- to 7-membered rings, whose ring structures include one to four heteroatoms. The term “heterocycle” may include heteroaryl groups, saturated heterocycles (e.g., cycloheteroalkyl) groups, or combinations thereof. The heterocycle may be a saturated molecule, or may comprise one or more double bonds. In some case, the heterocycle is a nitrogen heterocycle, wherein at least one ring comprises at least one nitrogen ring atom. The heterocycles may be fused to other rings to form a polycylic heterocycle. Thus, heterocycles also include bicyclic, tricyclic, and tetracyclic groups in which any of the above heterocyclic rings is fused to one or two rings independently selected from aryls, cycloalkyls, and heterocycles. The heterocycle may also be fused to a spirocyclic group.

Heterocycles include, for example, thiophene, benzothiophene, thianthrene, furan, tetrahydrofuran, pyran, isobenzofuran, chromene, xanthene, phenoxathiin, pyrrole, dihydropyrrole, pyrrolidine, imidazole, pyrazole, pyrazine, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, triazole, tetrazole, oxazole, isoxazole, thiazole, isothiazole, phenanthridine, acridine, pyrimidine, phenanthroline, phenazine, phenarsazine, phenothiazine, furazan, phenoxazine, pyrrolidine, oxolane, thiolane, oxazole, oxazine, piperidine, homopiperidine (hexamethyleneimine), piperazine (e.g., N-methyl piperazine), morpholine, lactones, lactams such as azetidinones and pyrrolidinones, sultams, sultones, other saturated and/or unsaturated derivatives thereof, and the like.

In some cases, the heterocycle may be bonded to a compound via a heteroatom ring atom (e.g., nitrogen). In some cases, the heterocycle may be bonded to a compound via a carbon ring atom. In some cases, the heterocycle is pyridine, imidazole, pyrazine, pyrimidine, pyridazine, acridine, acridin-9-amine, bipyridine, naphthyridine, quinoline, isoquinoline, benzoquinoline, benzoisoquinoline, phenanthridine-1,9-diamine, or the like.

The term “heteroaromatic” or “heteroaryl” as used herein refers to a mono-, bi-, or multi-cyclic aromatic ring system containing one or more heteroatoms, for example 1-3 heteroatoms, such as nitrogen, oxygen, and sulfur. Heteroaryls can also be fused to non-aromatic rings. In various embodiments, the term “heteroaromatic” or “heteroaryl,” as used herein except where noted, represents a stable 5- to 7-membered monocyclic, stable 9- to 10-membered fused bicyclic, or stable 12- to 14-membered fused tricyclic heterocyclic ring system which contains an aromatic ring that contains at least one heteroatom selected from the group consisting of N, O, and S. In some embodiments, at least one nitrogen is in the aromatic ring.

Heteroaromatics or heteroaryls can include, but are not limited to, a monocyclic aromatic ring, wherein the ring comprises 2-5 carbon atoms and 1-3 heteroatoms, referred to herein as “(C₂-C₅)heteroaryl.” Illustrative examples of monocyclic heteroaromatic (or heteroaryl) include, but are not limited to, pyridine (pyridinyl), pyridazine (pyridazinyl), pyrimidine (pyrimidyl), pyrazine (pyrazyl), triazine (triazinyl), pyrrole (pyrrolyl), pyrazole (pyrazolyl), imidazole (imidazolyl), (1,2,3)- and (1,2,4)-triazole ((1,2,3)- and (1,2,4)-triazolyl), pyrazine (pyrazinyl), pyrimidine (pyrimidinyl), tetrazole (tetrazolyl), furan (furyl), thiophene (thienyl), isoxazole (isoxazolyl), thiazole (thiazolyl), isoxazole (isoxazolyl), and oxazole (oxazolyl).

The term “bicyclic heteroaromatic” or “bicyclic heteroaryl” as used herein refers to a heteroaryl group fused to another aromatic or non-aromatic carbocylic or heterocyclic ring. Exemplary bicyclic heteroaromatics or heteroaryls include, but are not limited to 5,6- or 6,6-fused systems, wherein one or both rings contain heteroatoms. The term “bicyclic heteroaromatic” or “bicyclic heteroaryl” also encompasses reduced or partly reduced forms of fused aromatic system wherein one or both rings contain ring heteroatoms. The ring system may contain up to three heteroatoms, independently selected from oxygen, nitrogen, and sulfur.

Exemplary bicyclic heteroaromatics (or heteroaryls) include, but are not limited to, quinazoline (quinazolinyl), benzoxazole (benzoxazolyl), benzothiophene (benzothiophenyl), benzoxazole (benzoxazolyl), benzisoxazole (benzisoxazolyl), benzimidazole (benzimidazolyl), benzothiazole (benzothiazolyl), benzofurane (benzofuranyl), benzisothiazole (benzisothiazolyl), indole (indolyl), indazole (indazolyl), indolizine (indolizinyl), quinoline (quinolinyl), isoquinoline (isoquinolinyl), naphthyridine (naphthyridyl), phthalazine (phthalazinyl), phthalazine (phthalazinyl), pteridine (pteridinyl), purine (purinyl), benzotriazole (benzotriazolyl), and benzofurane (benzofuranyl). In some embodiments, the bicyclic heteroaromatic (or bicyclic heteroaryl) is selected from quinazoline (quinazolinyl), benzimidazole (benzimidazolyl), benzothiazole (benzothiazolyl), indole (indolyl), quinoline (quinolinyl), isoquinoline (isoquinolinyl), and phthalazine (phthalazinyl). In certain embodiments, the bicyclic heteroaromatic (or bicyclic heteroaryl) is quinoline (quinolinyl) or isoquinoline (isoquinolinyl).

The term “tricyclic heteroaromatic” or “tricyclic heteroaryl” as used herein refers to a bicyclic heteroaryl group fused to another aromatic or non-aromatic carbocylic or heterocyclic ring. The term “tricyclic heteroaromatic” or “tricyclic heteroaryl” also encompasses reduced or partly reduced forms of fused aromatic system wherein one or both rings contain ring heteroatoms. Each of the ring in the tricyclic heteroaromatic (tricyclic heteroaryl) may contain up to three heteroatoms, independently selected from oxygen, nitrogen, and sulfur.

Exemplary tricyclic heteroaromatics (or heteroaryls) include, but are not limited to, acridine (acridinyl), 9H-pyrido[3,4-b]indole (9H-pyrido[3,4-b]indolyl), phenanthridine (phenanthridinyl), pyrido[1,2-a]benzimidazole (pyrido[1,2-a]benzimidazolyl), and pyrido[1,2-b]indazole (pyrido[1,2-b]indazolyl).

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-22, 1-8, or 1-6 carbon atoms, referred to herein as (C₁-C₂₂)alkoxy, (C₁-C₈)alkoxy, or (C₁-C₆)alkoxy, respectively. Exemplary alkoxy groups include, but are not limited to methoxy and ethoxy.

The term “cycloalkoxy” as used herein refers to a cycloalkyl group attached to an oxygen.

The term “aryloxy” or “aroxy” as used herein refers to an aryl group attached to an oxygen atom. Exemplary aryloxy groups include, but are not limited to, aryloxys having a monocyclic aromatic ring system, wherein the ring comprises 6 carbon atoms, referred to herein as “(C₆)aryloxy.” The term “arylalkoxy” as used herein refers to an arylalkyl group attached to an oxygen atom. An exemplary aryalkyl group is benzyloxy group.

The term “amine” or “amino” as used herein refers to both unsubstituted and substituted amines, e.g., NRₐR_{b}R_{b′}, where Rₐ, R_{b}, and R_{b′} are independently selected from alkyl, alkenyl, alkynyl, aryl, arylalkyl, carbamate, cycloalkyl, haloalkyl, heteroaryl, heterocyclyl, and hydrogen, and at least one of the Rₐ, R_{b}, and R_{b′} is not hydrogen. The amine or amino can be attached to the parent molecular group through the nitrogen. The amine or amino also may be cyclic, for example any two of Rₐ, R_{b}, and R_{b′} may be joined together and/or with the N to form a 3- to 12-membered ring (e.g., morpholino or piperidinyl). The term amino also includes the corresponding quaternary ammonium salt of any amino group. Exemplary amines include alkylamine, wherein at least one of RₐR_{b}, or R_{b′} is an alkyl group, or cycloalkylamine, wherein at least one of RₐR_{b}, or R_{b′} is a cycloalkyl group.

The term “ammonia” as used herein refers to NH₃.

The term “aldehyde” or “formyl” as used herein refers to —CHO.

The term “acyl” term as used herein refers to a carbonyl radical attached to an alkyl, alkenyl, alkynyl, cycloalkyl, heterocycyl, aryl, or heteroaryl. Exemplary acyl groups include, but are not limited to, acetyl, formyl, propionyl, benzoyl, and the like.

The term “amide” as used herein refers to the form —NR_{c}C(O)(R_{d})— or —C(O)NR_{c}Rₑ, wherein R_{c}, R_{d}, and Rₑ are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{c}, R_{d}, or Rₑ. The amide also may be cyclic, for example R_{c} and Rₑ, may be joined to form a 3- to 12-membered ring, such as a 3- to 10-membered ring or a 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., -amide-COOH or salts such as -amide-COONa.

The term “arylthio” as used herein refers to an aryl group attached to an sulfur atom. Exemplary arylthio groups include, but are not limited to, arylthios having a monocyclic aromatic ring system, wherein the ring comprises 6 carbon atoms, referred to herein as “(C₆)arylthio.”

The term “arylsulfonyl” as used herein refers to an aryl group attached to a sulfonyl group, e.g., —S(O)₂-aryl-. Exemplary arylsulfonyl groups include, but are not limited to, arylsulfonyls having a monocyclic aromatic ring system, wherein the ring comprises 6 carbon atoms, referred to herein as “(C₆)arylsulfonyl.”

The term “carbamate” as used herein refers to the form —R_{f}OC(O)N(R_{g})—, —R_{f}OC(O)N(R_{g})Rₕ—, or —OC(O)NR_{g}Rₕ, wherein R_{f}, R_{g}, and Rₕ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of R_{f}, R_{g} and Rₕ are independently selected from aryl or heteroaryl, such as pyridinyl, pyridazinyl, pyrimidinyl, and pyrazinyl).

The term “carbonyl” as used herein refers to —C(O)—.

The term “carboxy” or “carboxylate” as used herein refers to Rⱼ—COOH or its corresponding carboxylate salts (e.g., Rⱼ—COONa), where Rⱼ can independently be selected from alkoxy, aryloxy, alkyl, alkenyl, alkynyl, amide, amino, aryl, arylalkyl, cycloalkyl, ether, haloalkyl, heteroaryl, and heterocyclyl. Exemplary carboxys include, but are not limited to, alkyl carboxy wherein Rⱼ is alkyl, such as —O—C(O)-alkyl. Exemplary carboxy also include aryl or heteoraryl carboxy, e.g. wherein Rⱼ is an aryl, such as phenyl and tolyl, or heteroaryl group such as pyridine, pyridazine, pyrmidine and pyrazine. The term carboxy also includes “carboxycarbonyl,” e.g. a carboxy group attached to a carbonyl group, e.g., —C(O)—COOH or salts, such as —C(O)—COONa.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “cyano” as used herein refers to —CN.

The term “ester” refers to the structure —C(O)O—, —C(O)O—Rᵢ—, —RⱼC(O)O—Rᵢ—, or —RⱼC(O)O—, where O is not bound to hydrogen, and Rᵢ and Rⱼ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rᵢ can be a hydrogen, but Rⱼ cannot be hydrogen. The ester may be cyclic, for example the carbon atom and Rⱼ, the oxygen atom and Rᵢ, or Rᵢ and Rⱼ may be joined to form a 3- to 12-membered ring. Exemplary esters include, but are not limited to, alkyl esters wherein at least one of R or R is alkyl, such as —O—C(O)-alkyl, —C(O)—O-alkyl-, and -alkyl-C(O)—O-alkyl-. Exemplary esters also include aryl or heteroaryl esters, e.g. wherein at least one of Rᵢ or Rⱼ is an aryl group, such as phenyl or tolyl, or a heteroaryl group, such as pyridine, pyridazine, pyrimidine or pyrazine, such as a nicotinate ester. Exemplary esters also include reverse esters having the structure —RⱼC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The term “ether” refers to the structure —RₖO—Rₗ—, where Rₖ and Rₗ can independently be alkyl, alkenyl, alkynyl, aryl, cycloalkyl, heterocyclyl, and ether. The ether can be attached to the parent molecular group through Rₖ or Rₗ. Exemplary ethers include, but are not limited to, alkoxyalkyl and alkoxyaryl groups. Ethers also includes polyethers, e.g., where one or both of Rₖ and Rₗ are ethers.

The terms “halo” or “halogen” or “hal” or “halide” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The terms “hydroxy” and “hydroxyl” as used herein refers to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rₘ (such as acetyl, —C(O)CH₃) or —Rₘ—C(O)—Rₙ—. The ketone can be attached to another group through Rₘ or Rₙ. Rₘ or Rₙ can be alkyl, alkenyl, alkynyl, cycloalkyl, heterocyclyl or aryl, or Rₘ or Rₙ can be joined to form, for example, a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “nitro” as used herein refers to —NO₂.

The term “nitrate” as used herein refers to NO₃⁻.

The term “perfluoroalkyl” as used herein refers to an alkyl group in which all of the hydrogen atoms have been replaced by fluorine atoms. Exemplary perfluoroalkyl groups include, but are not limited to, C₁-C₅ perfluoroalkyl, such as trifluoromethyl.

The term “perfluorocycloalkyl” as used herein refers to a cycloalkyl group in which all of the hydrogen atoms have been replaced by fluorine atoms.

The term “perfluoroalkoxy” as used herein refers to an alkoxy group in which all of the hydrogen atoms have been replaced by fluorine atoms.

The term “phosphate” as used herein refers to the structure —OP(O)O₂²⁻, —RₒOP(O)O₂²⁻, —OP(O)(OR_{q})O⁻, or —RₒOP(O)(ORₚ)O⁻, wherein Rₒ, Rₚ and R_{q} each independently can be alkyl, alkenyl, alkynyl, aryl, cycloalkyl, heterocyclyl, or hydrogen.

The term “sulfide” as used herein refers to the structure —R_{q}S—, where R_{q} can be alkyl, alkenyl, alkynyl, aryl, arylalkyl, cycloalkyl, haloalkyl, heteroaryl, heterocyclyl. The sulfide may be cyclic, for example, forming a 3 to 12-membered ring. The term “alkylsulfide” as used herein refers to an alkyl group attached to a sulfur atom.

The term “sulfinyl” as used herein refers to the structure —S(O)O—, —RᵣS(O)O—, —RᵣS(O)ORₛ—, or —S(O)ORₛ—, wherein Rᵣ and Rₛ can be alkyl, alkenyl, aryl, arylalkyl, cycloalkyl, haloalkyl, heteroaryl, heterocyclyl, hydroxyl. Exemplary sulfinyl groups include, but are not limited to, alkylsulfinyls wherein at least one of Rᵣ or Rₛ is alkyl, alkenyl, or alkynyl.

The term “sulfonamide” as used herein refers to the structure —(Rₜ)—N—S(O)₂—Rᵥ— or —Rₜ(Rᵤ)N—S(O)₂—Rᵥ, where Rₜ, Rᵤ, and Rᵥ can be, for example, hydrogen, alkyl, alkenyl, alkynyl, aryl, cycloalkyl, and heterocyclyl. Exemplary sulfonamides include alkylsulfonamides (e.g., where Rᵥ is alkyl), arylsulfonamides (e.g., where Rᵥ is aryl), cycloalkyl sulfonamides (e.g., where Rᵥ is cycloalkyl), and heterocyclyl sulfonamides (e.g., where Rᵥ is heterocyclyl).

The term “sulfonate” as used herein refers to a salt or ester of a sulfonic acid. The term “sulfonic acid” refers to R_{w}SO₃H, where R_{w} is alkyl, alkenyl, alkynyl, aryl, cycloalkyl, or heterocyclyl (e.g., alkylsulfonyl). The term “sulfonyl” as used herein refers to the structure RₓSO₂—, where Rₓ can be alkyl, alkenyl, alkynyl, aryl, cycloalkyl, and heterocyclyl (e.g., alkylsulfonyl). The term “alkylsulfonyl” as used herein refers to an alkyl group attached to a sulfonyl group. “Alkylsulfonyl” groups can optionally contain alkenyl or alkynyl groups.

The term “sulfonate” as used herein refers R_{w}SO₃⁻, where R_{w} is alkyl, alkenyl, alkynyl, cycloalkyl, aryl, heterocyclyl, hydroxyl, alkoxy, aroxy, or aralkoxy, where each of the alkyl, alkenyl, alkynyl, cycloalkyl, aryl, heteroaryl, alkoxy, aroxy, or aralkoxy optionally is substituted. Non-limiting examples include triflate (also known as trifluoromethanesulfonate, CF₃SO₃), benzenesulfonate, tosylate (also known as toluenesulfonate), and the like.

The term “thioketone” refers to the structure —R_{y}—C(S)—R_{z}—. The ketone can be attached to another group through R_{y} or R_{z}. R_{y} or R_{z} can be alkyl, alkenyl, alkynyl, cycloalkyl, heterocyclyl or aryl, or R_{y} or R_{z} can be joined to form a ring, for example, a 3- to 12-membered ring.

Each of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a functional group by which a molecule is identified, e.g., such that the “substituted” functional group becomes, through substitution, a different functional group. For example, a “substituted phenyl group” must still comprise the phenyl moiety and cannot be modified by substitution, in this definition, to become, e.g., a pyridine ring.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of the present teachings, the heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

As a non-limiting example, in various embodiments when one of the Rₐ, R_{b}, and R_{b′} in NRₐR_{b}R_{b′}, referred to herein as an amine or amino, is selected from alkyl, alkenyl, alkynyl, cycloalkyl, and heterocyclyl, each of the alkyl, alkenyl, alkynyl, cycloalkyl, and heterocyclyl independently can be optionally substituted with one or more substituents each independently selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents. In some embodiments when the amine is an alkyl amine or a cycloalkylamine, the alkyl or the cycloalkyl can be substituted with one or more substituents each independently selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. In certain embodiments when the amine is an alkyl amine or a cycloalkylamine, the alkyl or the cycloalkyl can be substituted with one or more substituents each independently selected from amino, carboxy, cyano, and hydroxyl. For example, the alkyl or the cycloalkyl in the alkyl amine or the cycloalkylamine is substituted with an amino group, forming a diamine.

As used herein, a “suitable substituent” refers to a group that does not nullify the synthetic or pharmaceutical utility of the compounds of the invention or the intermediates useful for preparing them. Examples of suitable substituents include, but are not limited to: (C₁-C₂₂), (C₁-C₈), (C₁-C₆), or (C₁-C₄) alkyl, alkenyl or alkynyl; (C₆-C₂₂), (C₆-C₁₈), (C₆-C₁₄), or (C₆-C₁₀) aryl; (C₂-C₂₁), (C₂-C₁₇), (C₂-C₁₃), or (C₂-C₉) heteroaryl; (C₃-C₂₂), (C₃-C₁₂), or (C₃-C₈) cycloalkyl; (C₁-C₂₂), (C₁-C₈), (C₁-C₆), or (C₁-C₄) alkoxy; (C₆-C₂₂), (C₆-C₁₈), (C₆-C₁₄), or (C₆-C₁₀) aryloxy; —CN; —OH; oxo; halo; carboxy; amino, such as —NH((C₁-C₂₂), (C₁-C₈), (C₁-C₆), or (C₁-C₄) alkyl), —N((C₁-C₂₂), (C₁-C₈), (C₁-C₆), or (C₁-C₄) alkyl)₂, —NH((C₆)aryl), or —N((C₆-C₁₀) aryl)₂; formyl; ketones, such as —CO((C₁-C₂₂), (C₁-C₈), (C₁-C₆), or (C₁-C₄) alkyl), —CO(((C₆-C₁₀) aryl) esters, such as —CO₂((C₁-C₂₂), (C₁-C₈), (C₁-C₆), or (C₁-C₄) alkyl) and —CO₂((C₆-C₁₀) aryl). One of skill in art can readily choose a suitable substituent based on the stability and pharmacological and synthetic activity of the compound of the invention.

The term “pharmaceutically acceptable counter ion” refers to a pharmaceutically acceptable anion or cation. In various embodiments, the pharmaceutically acceptable counter ion is a pharmaceutically acceptable ion. For example, the pharmaceutically acceptable counter ion is selected from citrate, malate, acetate, oxalate, chloride, bromide, iodide, nitrate, sulfate, bisulfate, phosphate, acid phosphate, isonicotinate, acetate, lactate, salicylate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)). In some embodiments, the pharmaceutically acceptable counter ion is selected from chloride, bromide, iodide, nitrate, sulfate, bisulfate, phosphate, acid phosphate, citrate, malate, acetate, oxalate, acetate, and lactate. In particular embodiments, the pharmaceutically acceptable counter ion is selected from chloride, bromide, iodide, nitrate, sulfate, bisulfate, and phosphate.

The term “pharmaceutically acceptable salt(s)” refers to salts of acidic or basic groups that may be present in compounds used in the present teachings. Compounds included in the present teachings that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitart rate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present teachings that include an amino moiety may form pharmaceutically acceptable salts with various amino acids, in addition to the acids mentioned above. Compounds included in the present teachings, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

Unless otherwise specified, the chemical groups include their corresponding monovalent, divalent, trivalent, and tetravalent groups. For example, methyl includes monovalent methyl (—CH₃), divalent methyl (—CH₂—, methylyl), trivalent methyl

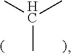

and tetravalent methyl

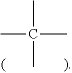

Unless otherwise specified, all numbers expressing quantities of ingredients, reaction conditions, and other properties or parameters used in the specification and claims are to be understood as being modified in all instances by the term “about.” Accordingly, unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques. For example, the term “about” can encompass variations of ±10%, ±5%, ±2%, ±1%, ±0.5%, or ±0.1% of the numerical value of the number which the term “about” modifies. In various embodiments, the term “about” encompasses variations of ±5%, ±2%, ±1%, or ±0.5% of the numerical value of the number. In some embodiments, the term “about” encompasses variations of ±5%, ±2%, or ±1% of the numerical value of the number. In certain embodiments, the term “about” encompasses variations of ±5% of the numerical value of the number. In certain embodiments, the term “about” encompasses variations of ±2% of the numerical value of the number. In certain embodiments, the term “about” encompasses variations of ±1% of the numerical value of the number.

All numerical ranges herein include all numerical values and ranges of all numerical values within the recited range of numerical values. As a non-limiting example, (C₁-C₆) alkyls also include any one of C₁, C₂, C₃, C₄, C₅, C₆, (C₁-C₂), (C₁-C₃), (C₁-C₄), (C₁-C₅), (C₂-C₃), (C₂-C₄), (C₂-C₅), (C₂-C₆), (C₃-C₄), (C₃-C₅), (C₃-C₆), (C₄-C₅), (C₄-C₆), and (C₅-C₆) alkyls.

Further, while the numerical ranges and parameters setting forth the broad scope of the disclosure are approximations as discussed above, the numerical values set forth in the Examples section are reported as precisely as possible. It should be understood, however, that such numerical values inherently contain certain errors resulting from the measurement equipment and/or measurement technique.

The present teachings generally provide compounds, compositions, and methods of using the compounds or compositions.

### I. Compounds

In various embodiments provided herein, a platinum (IV) compound (Pt(IV)) includes a suitable reacting group for reacting with a functional group on a protein, engineered protein, antibody, antibody fragment, peptide, agonist, antagonist, aptamer or any moiety which may be capable of recognizing a selected target cell population, and/or derivatives/analogs/mimics thereof. The reacting group possesses protein-conjugating properties, i.e., it binds covalently to the protein. For example, the reacting group can be introduced by a linker between the reacting group and platinum. In various embodiments, one of or both the axial ligands of platinum each comprises one or more reacting groups. In some embodiments, one or more of the four equatorial ligands of platinum each comprises one or more reacting groups.

In some embodiments, the protein is albumin and/or derivatives/analogs/mimics thereof.

In some embodiments, the reacting group is a Michael acceptor such as maleimide.

The Pt(IV)M compound of the present invention has a general structure of:

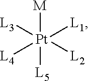

wherein M is a ligand comprising any suitable reacting group for reacting with a functional group on a protein; L₁, L₂, L₃, L₄, and L₅ may be any suitable ligand for Pt. M may further comprise a linker between the reacting group and platinum. M may be at the any of the two axial sites, as well as the four equatorial sites of platinum. “Ligand”, as used herein, means an ion, molecule, or functional group that binds to a metal atom to form a coordination complex. The bonding between a ligand and a metal atom may involve donation of one or more of the ligand's electron pairs. L₁, L₂, L₃, L₄ and L₅ can be the same or different. They may each be selected from a group comprising at least one of ammonia, an amine, a halide, a heterocycle including at least one nitrogen, an aryl group, or a leaving group, any being optionally substituted, or, any two or three of L₁, L₂, L₃, L₄ and L₅ can be joined together to form a bidentate ligand or tridentate ligand, any being optionally substituted.

As used herein, a “leaving group” is given its ordinary meaning in the art and refers to an atom or a group capable of being displaced by a nucleophile. Examples of suitable leaving groups include, but are not limited to, halides (such as chloride, bromide, and iodide), alkanesulfonyloxy, arenesulfonyloxy, alkyl-carbonyloxy (e.g., acetoxy, carboxylate), arylcarbonyloxy, mesyloxy, tosyloxy, trifluoromethane-sulfonyloxy, aryloxy, methoxy, N,O-dimethylhydroxylamino, pixyl, oxalato, malonato, and the like. A leaving group may also be a bidentate, tridentate, or other multidentate ligand. In some embodiments, the leaving group is a halide or carboxylate. In some embodiments, the leaving group is chloride.

### Ligand M Comprising a Reacting Group

The reacting group comprised on ligand M may be conjugated to a peptide, antibody mimetic, nucleic acid (e.g. aptamer), polypeptide (e.g. antibody), glycoprotein, small molecule, carbohydrate, or lipid.

In some embodiments, the reacting group of the Pt(IV)M compound is an albumin-conjugating group. For example, the reacting group may bind to an amino, a hydroxy or a thiol group of albumin. Non-limiting examples of an albumin-conjugating group include chemical groups such as a maleimide group, a halogenacetamide group, a halogenacetate group, a pyridylthio group, a vinylcarbonyl group, an aziridin group, a disulfide group, a substituted or unsubstituted acetylene group, a thiol group, a vinyl acetylene group, an epoxide, an acetylene group, and a hydroxysuccinimide ester group. In one embodiment, the reacting group comprised on ligand M is a maleimide or its derivative/analog.

Any albumin-binding group disclosed in US 2015/0023912 to Kratz et al., the contents of which are incorporated herein by reference in their entirety, may be used as a reacting group of the Pt(IV)M compound. Kratz et al. teaches that the albumin-binding group may further include functional groups such as —COOH or —SO₃H, that can be activated by standard coupling agents such as dicyclocarbodiimides, acid chlorides, or peptide coupling reagents (e.g., BOP, HATU, PyBOP). Further examples of the albumin-binding group include compounds or derivatives of phthalocyanines, coumarins, flavonoids, tetracyclines, naphthalenes, aryl- and heteroarylcarboxylic acids, lipids and fatty acids, for example long-chain fatty acids such as C10-C20 fatty acids or C10-C20 alkyl amines, cyclic or linear tetrapyrroles and organometallic compounds thereof, for example porphyrins and protoporphyrins (such as bilirubin, hematin and derivatives thereof), aromatic acid derivatives substituted with 2-5 halogen atoms (e.g. F, Cl, Br or I) such as iophenoxic acid, organic dyes, for example Evans blue and bromcresol dyes such as bromcresol green and bromcresol purple, and the tryptophan and thyroxine analog compounds as well as derivatives of the above-indicated classes of compounds. Furthermore, according to the present invention, said organic dyes and their derivatives used as albumin-binding groups can be chemically modified or derivatized before or after binding to the therapeutically and/or diagnostically active substance or to the spacer molecule, whereby however the binding behavior to albumin is maintained as compared with the unmodified compound. As an example, a dye used as an albumin-binding group, e.g. an azo dye, may be derivatized by the above-indicated chemical modification, for example by reduction of the azo group or by replacement of the azo group by a C—C single bond or a C—C double bond, in such a way that it is no longer colored.

The albumin-binding group of the Pt(IV)M compound binds to serum albumin in situ after administration. The term ‘in situ’, as used herein, describes the binding of Pt(IV)M to serum albumin inside a subject to which the Pt(IV)M compound is administered.

In some embodiments, a Pt(IV)M compound of the present invention has Formula I:

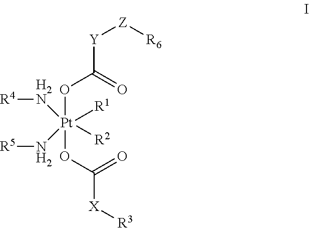

or a pharmaceutically acceptable salt thereof, wherein:
X and Y are independently selected from NH, alkyl and aryl;
R¹ and R² each is Cl, or R¹ and R² are joined to form an oxalate;
R³ is hydrogen, alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl-ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl;
R⁴ and R⁵ are each H or together constitute a cyclohexyl ring;
Z is alternatively absent or alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl; and
R⁶ is a suitable reacting group for reacting with a functional group on a protein such as but not limited to:

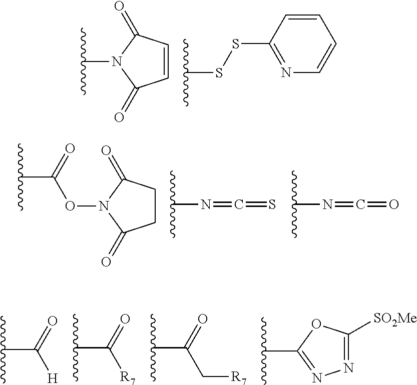

where R⁷ is Cl, Br, F, mesylate, tosylate, O-(4-nitrophenyl), O-pentafluorophenyl. The reacting group can also be an activated disulfide group, a vinylcarbonyl group, a vinyl acetylene group, an epoxide, an aziridine group or an acetylene group. The groups may be substituted, where appropriate.

An embodiment of the invention is a compound or a pharmaceutically acceptable salt thereof wherein X together with R³ is selected from the group consisting of:

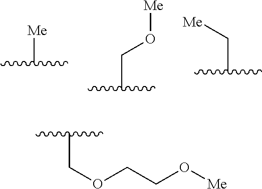

In some embodiments, the reacting group is a maleimide. Such compounds may be referred to herein as “monomaleimide compounds”, i.e., Pt(IV)M monomaleimide compounds. As used herein, “monomaleimide compounds” are compounds with a single maleimide group. The monomaleimide compound has Formula II:

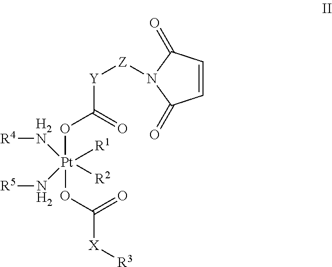

or a pharmaceutically acceptable salt thereof, wherein:
X and Y are independently selected from NH, alkyl and aryl;
R¹ and R² each is Cl, or R¹ and R² are joined to form an oxalate;
R³ is hydrogen, alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl-ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl;
R⁴ and R⁵ are each H or together constitute a cyclohexyl ring; and
Z is alternatively absent or alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

Not willing to be bound to any theory, the unsymmetrical nature of Pt(IV)M monomaleimide compounds allows for the modulation of platinum drug release.

Another embodiment of the invention is a maleimide compound or a pharmaceutically acceptable salt thereof wherein Y together with Z and the maleimide is selected from the group consisting of:

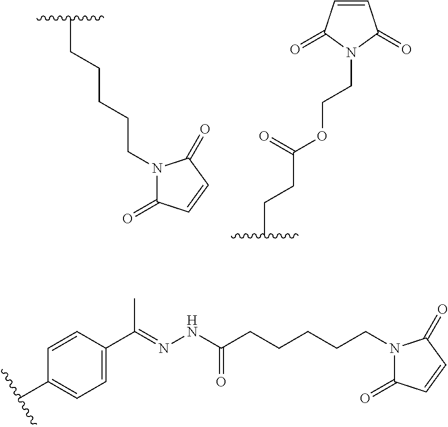 [SMILES: *CCCCCN1C(=O)C=CC1=O ; *CCC(=O)OCCN1C(=O)C=CC1=O ; *c1ccc(/C(C)=N/NC(=O)CCCCCN2C(=O)C=CC2=O)cc1]

Another embodiment of the invention is a maleimide compound having Formula IIa:

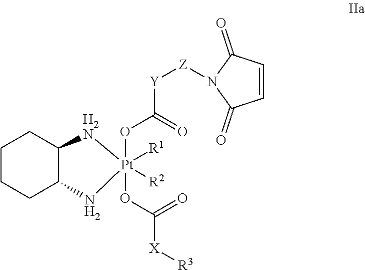

or a pharmaceutically acceptable salt thereof, wherein:
X and Y are independently selected from NH, alkyl and aryl;
R¹ and R² each is Cl, or R¹ and R² are joined to form an oxalate;
R³ is hydrogen, alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl-ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl; and
Z is alternatively absent or alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

Another embodiment of the invention is a maleimide compound having Formula IIb:

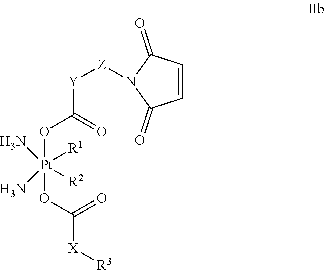

or a pharmaceutically acceptable salt thereof, wherein:
X and Y are independently selected from NH, alkyl and aryl;
R¹ and R² each is Cl, or R¹ and R² are joined to form an oxalate;
R³ is hydrogen, alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl-ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl; and
Z is alternatively absent or alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

A non-limiting example of a Pt(IV)M compound of the invention is a compound selected from the group consisting of the compounds listed:

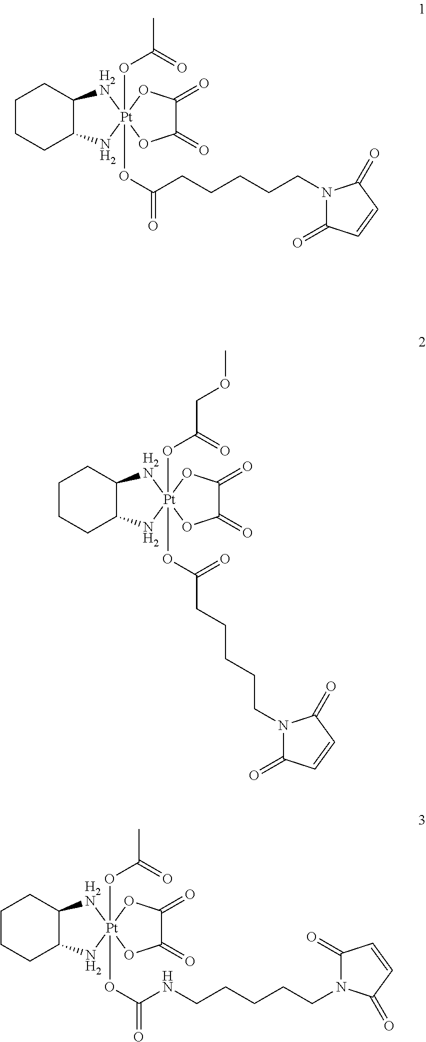

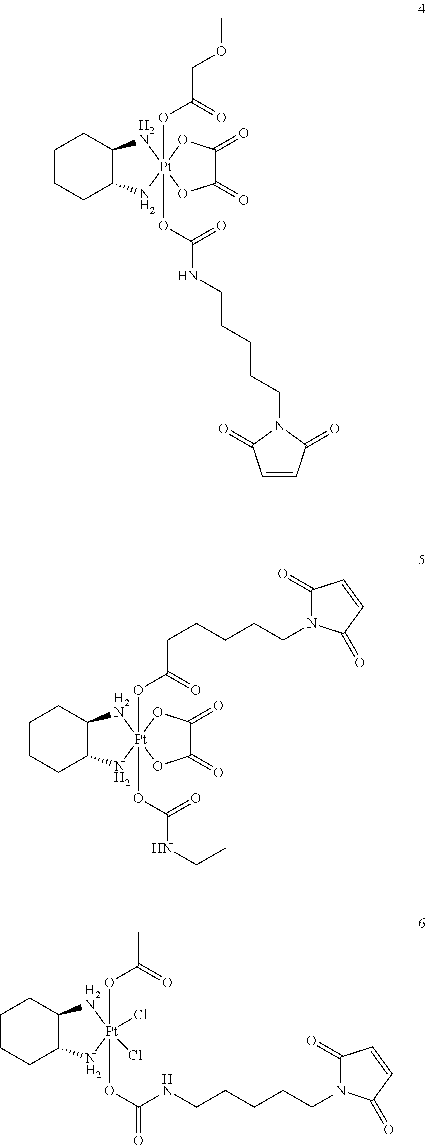

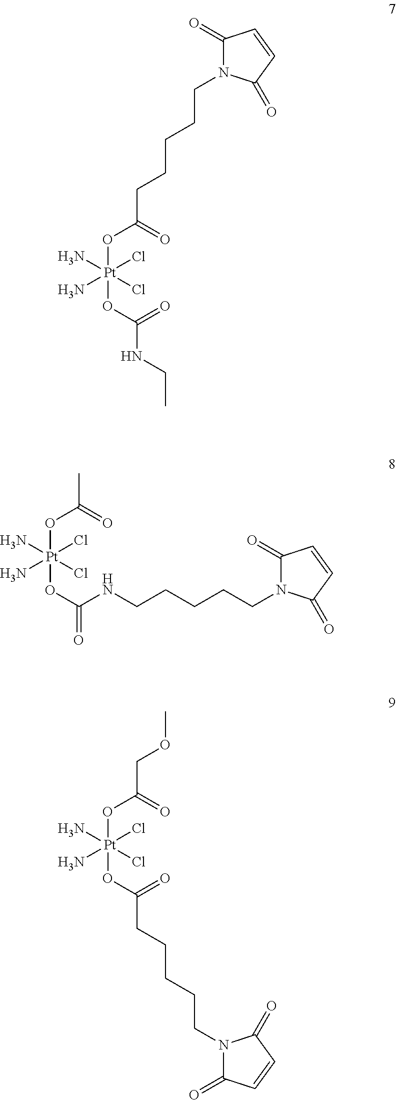

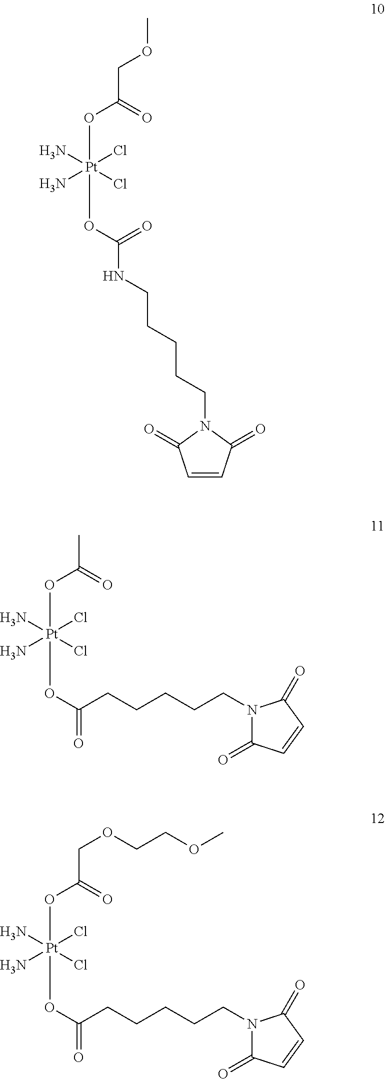

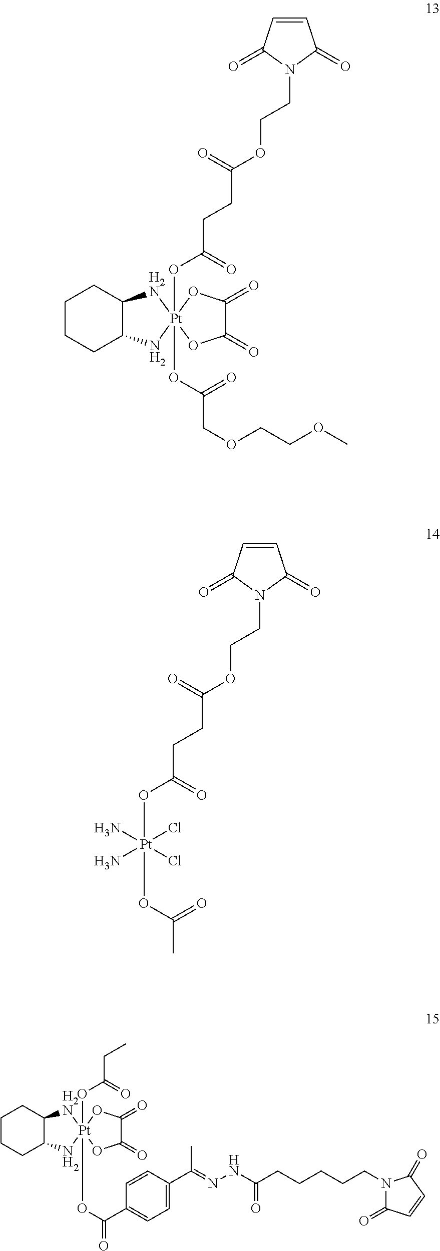

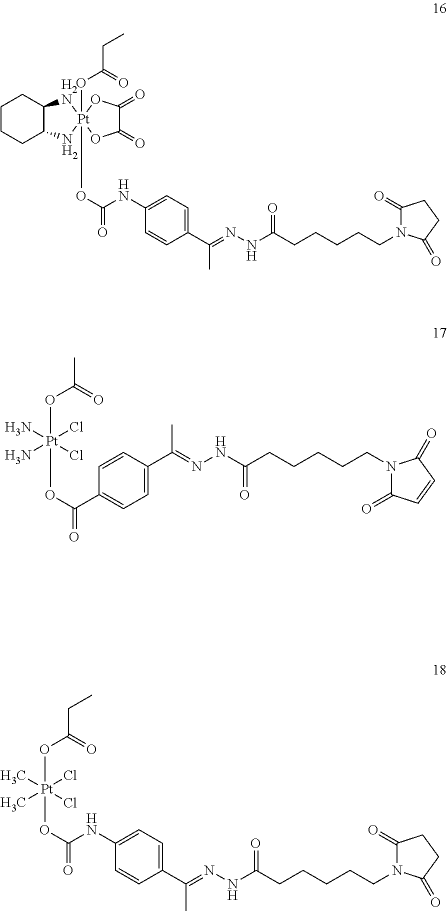

Another non-limiting example of a Pt(IV)M compound of the invention is a compound selected from the group consisting of the compounds listed:

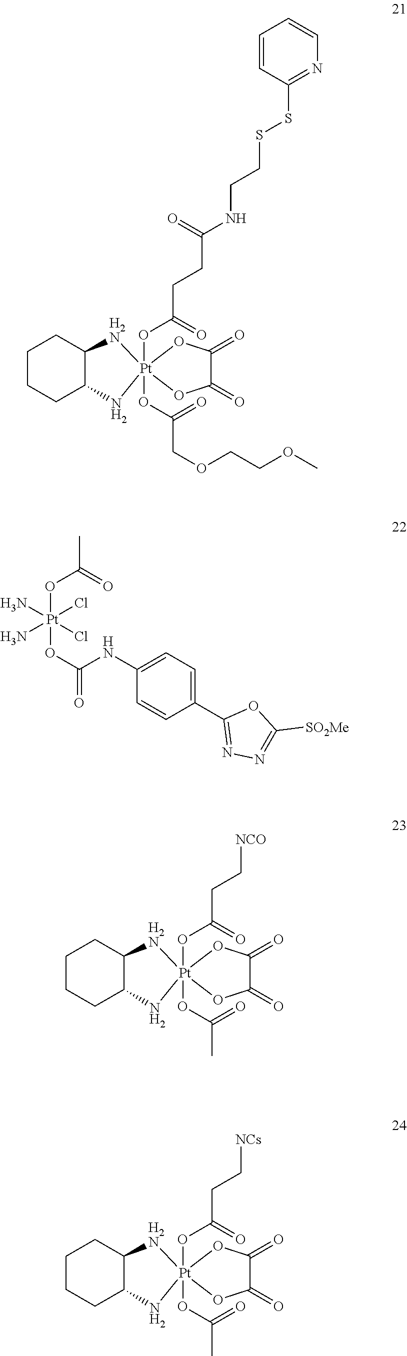

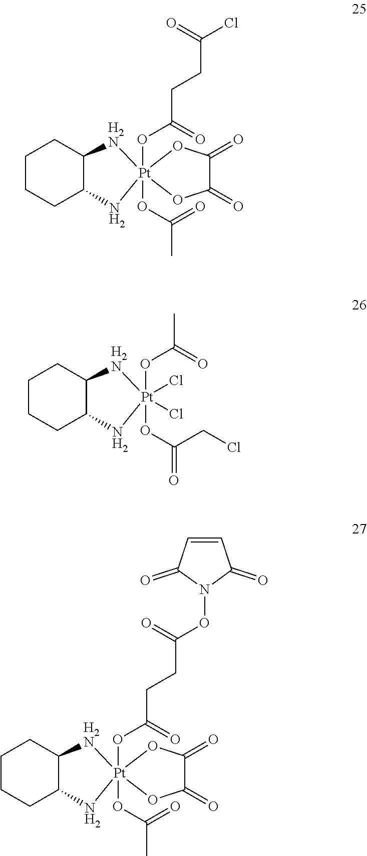

In some embodiments, the Pt(IV)M compound further comprises at least one active agent as a ligand of Pt(IV). The active agent may be bound to Pt(IV) with any suitable linker. The linker may be a cleavable linker that can be cleaved hydolytically, reductively, enzymatically, or in a pH-dependent manner.

The Pt(IV)M compound comprising at least one active agent has a general structure of:

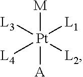

wherein M is a ligand comprising any suitable group for reacting with a function group on a protein as discussed above; A is a ligand comprising an active agent; and L₁, L₂, L₃, and L₄ may be any suitable ligand for Pt. In some embodiments, at least one of L₁, L₂, L₃, and L₄ may also be an active agent. M may be an albumin-binding group as discussed above. L₁, L₂, L₃, and L₄ can be the same or different. They may each be a group comprising at least one of ammonia, an amine, a heterocycle including at least one nitrogen, an aryl group, or a leaving group, any being optionally substituted, or, any two or three of L₁, L₂, L₃, and L₄ can be joined together to form a bidentate ligand or tridentate ligand, any being optionally substituted. M may further comprise a linker between the reacting group and platinum. M may be at the any of the two axial sites, as well as the four equatorial sites of platinum. A may further comprise a linker between the active agent and platinum. A may be at the any of the two axial sites, as well as the four equatorial sites of platinum.

The active agent may be bound to Pt(IV) with any suitable linker either directly or through a spacer group. There is no specific restriction concerning the positions of the active agent and the reacting group relative to Pt(IV), as long as the functions of the active agent and the reacting group are not negatively affected. In some embodiments, the active agent may be at axial position of Pt(IV). In some embodiments, the active agent may be at equatorial position of Pt(IV). In some embodiments, the reacting group may be at axial position of Pt(IV). In some embodiments, the reacting group may be at equatorial position of Pt(IV).

Not willing to be bound to any theory, in the reducing environment of the cytoplasm of tumor cells, Pt(IV) complexes can be reduced to Pt(II) complexes. Therefore, the active agent may be released. In some embodiments, the Pt(II) complexes may themselves be an active agent. In some embodiments, the Pt(II) complexes is not in sufficient quantity to be therapeutically effective.

In some embodiments, the Pt(IV)M compound has a Formula A of

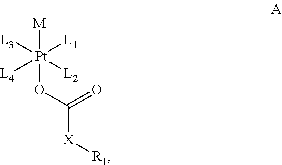

wherein X is selected from NH, alkyl and aryl;
R1 comprises an active agent;
L₁, L₂, L₃ and L₄ are independently any suitable ligand for Pt;
M is a ligand comprising a suitable reacting group for reacting with a functional group on a protein such as but not limited to:

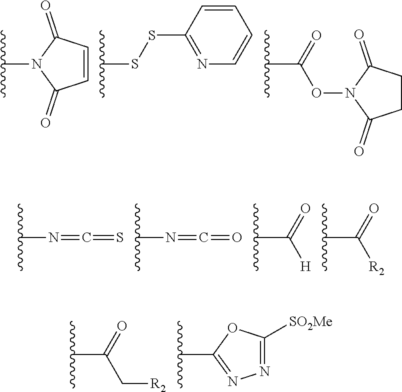

where R2 is Cl, Br, F, mesylate, tosylate, O-(4-nitrophenyl), O-pentafluorophenyl. The reacting group M can also be an activated disulfide group, a vinylcarbonyl group, a vinyl acetylene group, an epoxide, an aziridine group or an acetylene group. The groups may be substituted, where appropriate.

In some embodiments, the Pt(IV)M compound has a Formula A1 of

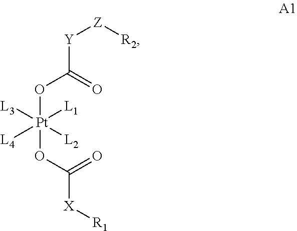

wherein X and Y are independently selected from NH, alkyl and aryl;
Z is alternatively absent or alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl;
L₁, L₂, L₃ and L₄ are independently any suitable ligand for Pt;
R1 is an active agent; and
R2 is a suitable reacting group for reacting with a functional group on a protein such as but not limited to:

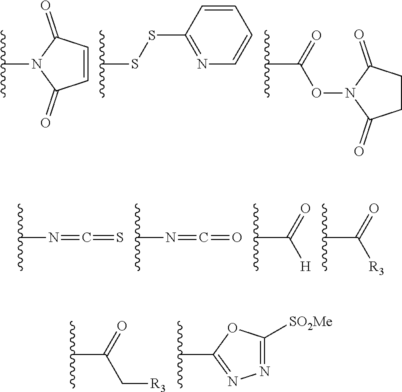

where R3 is Cl, Br, F, mesylate, tosylate, O-(4-nitrophenyl), O-pentafluorophenyl. The reacting group can also be an activated disulfide group, a vinylcarbonyl group, a vinyl acetylene group, an epoxide, an aziridine group or an acetylene group. The groups may be substituted, where appropriate.

In some embodiments, the Pt(IV)M compound has a Formula A2 of

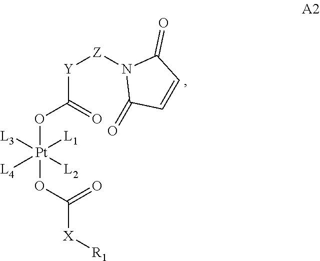

wherein X and Y are independently selected from NH, alkyl and aryl;
Z is alternatively absent or alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl;
L₁, L₂, L₃ and L₄ are independently any suitable ligand for Pt; and
R1 is an active agent.
Y together with Z and the maleimide may form a group selected from the group consisting of:

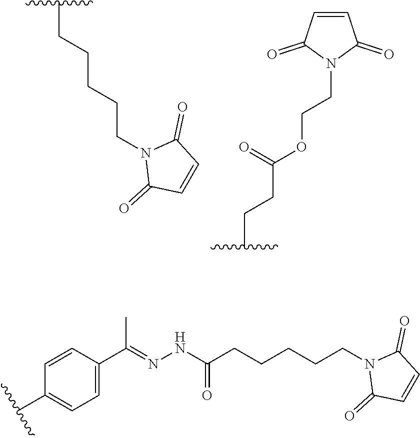 [SMILES: *CCCCCN1C(=O)C=CC1=O ; *CCC(=O)OCCN1C(=O)C=CC1=O ; *c1ccc(/C(C)=N/NC(=O)CCCCCN2C(=O)C=CC2=O)cc1]

### Active Agents

Ligand A of the Pt(IV) compounds may comprise at least one active agent. The at least one active agent may be a therapeutic, prophylactic, diagnostic, or nutritional agent. It may be a small molecule, protein, peptide, lipid, glycolipid, glycoprotein, lipoprotein, carbohydrate, sugar, or nucleic acid.

In some embodiments, the active agent may comprise a chemotherapeutic agent, antibiotic, antimicrobial, growth factor and combinations thereof.

In some embodiments, the active agent may be selected from a biomolecule, bioactive agent, small molecule, drug, prodrug, drug derivative, protein, peptide, vaccine, adjuvant, imaging agent (e.g., a fluorescent moiety) or polynucleotide.

Certain therapeutic agents are capable of preventing the establishment or growth (systemic or local) of a tumor or infection. Examples include boron-containing compounds (e.g., carborane), chemotherapeutic nucleotides, drugs (e.g., antibiotics, antivirals, antifungals), enediynes (e.g., calicheamicins, esperamicins, dynemicin, neocarzinostatin chromophore, and kedarcidin chromophore), heavy metal complexes (e.g., cisplatin), hormone antagonists (e.g., tamoxifen), non-specific (non-antibody) proteins (e.g., sugar oligomers), oligonucleotides (e.g., antisense oligonucleotides that bind to a target nucleic acid sequence (e.g., mRNA sequence)), peptides, photodynamic agents (e.g., rhodamine 123), radionuclides (e.g., I-131, Re-186, Re-188, Y-90, Bi-212, At-211, Sr-89, Ho-166, Sm-153, Cu-67 and Cu-64), toxins (e.g., ricin), and transcription-based pharmaceuticals. In certain embodiments for treating or preventing the establishment or growth of a tumor, the therapeutic agent may be a small molecule, radionuclide, toxin, hormone antagonist, heavy metal complex, oligonucleotide, chemotherapeutic nucleotide, peptide, non-specific (non-antibody) protein, a boron compound or an enediyne.

In other embodiments for treating or preventing the establishment or growth of a bacterial infection, the therapeutic agent may be an antibiotic, radionuclide or oligonucleotide. In still other embodiments for treating or preventing the establishment or growth of a viral infection, the therapeutic agent may be an antiviral compound, radionuclide or oligonucleotide. In yet other embodiments for treating or preventing the establishment or growth of a fungal infection, the therapeutic agent may be an antifingal compound, radionuclide or oligonucleotide.

In some embodiments, the active agent is an anti-cancer drug such as 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cabazitaxel, cactinomycin, calcipotriol, calphostin C, calusterone, camptothecin, camptothecin derivatives, canarypox IL-2, capecitabine, caracemide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castano spermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, larotaxel, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, 06-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum(IV) complexes, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxy ethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone Bl, ruboxyl, safingol, safingol hydrochloride, saintopin, sarcnu, sarcophytol A, sargramostim, SDI 1 mimetics, semustine, senescence derived inhibitor 1, sense oligonucleotides, siRNA, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride.

In some embodiments, the active agent may be any chemotherapeutic agent anti-infective agent disclosed in US 2014/0187501 to Bilodeau et al, the contents of which are incorporated herein by reference in their entirety. In some embodiments, the active agent is an antigen or adjuvant, radioactive or imaging agent (e.g., a fluorescent moiety) or polynucleotide. In some embodiments the active agent is an organometallic compound. For example, the active agent may be Cabazitaxel, Gemcitabine, Docetaxel, Paclitaxel, Etoposide, 5-FU, Leucovorin, Irinotecan, Cetuximab, Capecitabine, Methotrexate, Vinblastine, or Doxorubicin.

In accordance with the present invention, the active agent may be an active agent that can boost or provoke an anti-cancer immune response in a subject. Immunotherapy is an advantageous strategy to treat cancer. Any compound that can provoke and/or enhance an immune response to destroy tumor cells in a subject may be included in the Pt(IV)M compounds. Such active agents may be tumor associated antigens (TAAs), antigen epitopes including antigen peptides presented by either MHC (major histocompatibility complex) class I or MHC class II molecules; cytokines, chemokines, other immunomodulators, T cell receptors (TCRs), CD (cell differentiation molecules) antigens, antibodies, cytotoxic agents, cell adhesion molecules and any components that are involved in an immune response; or variants thereof. The active agent may be a protein including a peptide, a nucleic acid, a sugar, a lipid, a lipoprotein, a glucoprotein, a glycolipid, or a small molecule.

The initiation of an immune response against diseased tumor cells involves presenting a tumor specific antigen to the immune system. It has been known that tumor cells express specific antigens that are not normally expressed by normal cells. Many tumor associated antigens (TAAs) have been identified and antigenic peptides (epitopes) (either MHC class I specific or MHC class II specific) are isolated that can specifically activate an immune response (e.g., cytotoxic T lymphocyte response/CTL response) to attack abnormal tumor cells and promote their lysis in vivo. TAAs and epitope peptides derived from TAAs can be selected as antigens to selectively stimulate cytotoxic T lymphocyte (CTL) response. The ability of a TAA or a TAA peptide to induce CTL response depends on its ability to bind to specific MHC molecules and its ability to break immune tolerance.

In accordance with the present invention, a the active agent may be a TAA or an antigenic peptide (epitope) derived from a TAA. An antigenic peptide may be a CD8+ T cell epitope that binds to specific MHC (HLA in human) class I molecules with a high affinity. An antigenic peptide may be a CD4+ T cell epitope that binds to specific MEC (HLA in human) class II molecules with a high affinity. The antigenic peptide may be about 5 to 50 amino acids in length. The antigenic peptide may be greater than 5 amino acids in length, or greater than 10 amino acids in length, or greater than 15 amino acids in length, or greater than 20 amino acids in length, or greater than 25 amino acids in length, or greater than 30 amino acids in length, or greater than 35 amino acids in length, or greater than 40 amino acids in length, or greater than 45 amino acids in length. For example, the antigenic peptide may contain 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. It is generally preferable that the antigenic peptide be as small as possible while still maintaining substantially all of the immunologic activity of the native protein. In some aspects, the HLA class I binding antigenic peptides (epitopes) may have a length of about 6 to about 15 amino acid residues, for example, 6, 7, 8, 9, 10, 11, 12, 13, 14 or 15. In other aspects, the HLA class II binding peptides (epitopes) may have about 6 to about 30 amino acid residues, e.g., 6, 7, 8, 9, 10, 11, 12, 13, 14, 15, 16, 17, 18, 19, 20, 21, 22, 23, 24, 25, 26, 27, 28, 29 or 30 amino acids, preferably to between about 13 and about 20 amino acids, e.g., 13, 14, 15, 16, 17, 18, 19 or 20 amino acids.

TAAs or TAA derived antigenic peptides may be delivered directly to activate T cells through the targeting moieties of the conjugate. Conjugates of the present invention comprising one or more TAAs and/or antigenic peptides derived from TAAs may provide vaccine platforms that can enhance immunogenicity and reduce toxicity such as autoimmune toxicity.

A TAA active agent may be an oncofetal antigen that is typically only expressed at different stages during the development of the fetus and in cancerous somatic cells. Many proteins are normally expressed during fetal development but are transcriptionally repressed after birth or at early stage of infancy, therefore are not present, or are expressed in significantly lower levels in the corresponding normal adult tissue. Some of these developmental proteins are reexpressed in certain tumor cells and become oncofetal antigens. The oncofetal antigens have the potential to be used as tumour markers for diagnosis, treatment monitoring, follow-up after therapy and/or ultimately as targets for specific therapy of malignancy. Examples of oncoferal antigens may include, but are not limited to CEA (carcinoembryonic antigen) in colorectal carcinorma, iLRP/OFA (immature laminin receptor protein/oncofetal antigen) in renal cell carcinoma (RCC), TAG-72 (tumor asscociated glycoprotein-72) in prostate carcinoma, AFP (alpha-fetoprotein) in hepatocellular carcinoma (HCC), ROR1 (a receptor tyrosine kinase) in many malignant cells such as brain tumors, sperm protein 17, HMGA2 (high mobility group A2) in ovarian carcinoma, oncofetal H19, CR-1 (Cripto-1, a member of epidermal growth factor (EGF)-CFC family), trophoblast glycoprotein precursor and GPC-3 (Glypican-3, a member of heparan sulphate proteoglycans) in HCC. Some examples of T cell epitope peptides derived from oncofetal antigens may be used as payloads, such as those peptides disclosed in U.S. Pat. Nos. 7,718,762; 7,968,097; 7,994,276; 8,080,634; 8,669,230; 8,709,405; and U.S. patent publication NO: 2007/0049960; each of which is incorporated herein by reference in their entirety.

A TAA active agent may be an oncoviral antigen that is enocoded by tumorigenic transforming viruses (also called oncogenic viruses). Oncogenic viruses, when they infect host cells, can insert their own DNA (or RNA) into that of the host cells. When the viral DNA or RNA affects the host cell's genes, it can push the cell toward becoming cancer. Oncogenic viruses include, but are not limited to, RNA viruses, such as Flaviviridae and Retroviridae, and DNA viruses, such as Hepadnaviridae, Papovaviridae, specifically Papillomaviruses, Adenoviridae, Herpesviridae, and Poxviridae. Some examples of commonly known oncoviruses include human papilloma viruses (HPVs) which are main causes of cervical cancer, Epstein-Barr virus (EBV) which may cause nasopharyngeal cancer, certain types of fast-growing lymphomas (e.g., Burkitt lymphoma) and stomach cancer, hepatitis B, C and D viruses (HBV, HCV and HDV) in hepatocellular carcinoma (HCC), human immunodeficiency virus (HIV) which increases the risk of getting many typese of cancer (e.g., liver cancer, anal cancer and Hodgkin cacner), Kaposi sarcoma herpes virus (KSHV; also known as human herpes virus 8 (HHV8)) which is linked to lymphoma, human T-lymphotrophic virus (HTLV-1) and merkel cell polymavirus (MCV).

A TAA payload may be a post-translationally altered antigen due to tumor-associated alterations in glycosylation, and other posttranslation modifications. Some examples may include MUC1 in colorectal carcinoma.

A TAA payload may be a tumor specific transplantation antigen (TSTA).

Some examples of antigenic peptides and their corresponding genes/proteins, HLA subtypes to which an antigenic peptide binds and tumors associated with them are listed in Vigneron et al., Database of T cell defined human tumor antigens: the 2013 update, Cancer Immu. 2013, 13: 15, the contents of which are incorporated herein by reference in their entirety.

The active agent A of the Pt(IV)M compound comprises a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the Pt(IV)M compound.

### Linker

A linker may be used to attach the active agent to Pt(IV). In some embodiments, the linker may be a cleavable linker that releases the active agent upon delivery to a target cell. The cleavable linker may be cleaved physically or chemically.

In one embodiment, the linker can be a C1-C10 straight chain alkyl, C1-C10 straight chain O-alkyl, C1-C10 straight chain substituted alkyl, C1-C10 straight chain substituted O-alkyl, C4-C13 branched chain alkyl, C4-C13 branched chain O-alkyl, C2-C12 straight chain alkenyl, C2-C12 straight chain O-alkenyl, C3-C12 straight chain substituted alkenyl, C3-C12 straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C3 straight chain alkyl or a ketone.

In some embodiments, the linkers disclosed in US 2015/0023912 to Kratz et al., the contents of which are incorporated herein by reference in their entirety, may be used. For example, the linkers may be cleaved physically by light, radioactive emission or heat. The linkers may also be cleaved chemically by redox reactions, hydrolysis, pH-dependent cleavage or cleavage by enzymes. The cleavage of the linkers can be performed in vivo, e.g., in the body of a patient.

The linker can be any linker moiety disclosed in US 2014/0187501 to Bilodeau et al., the contents of which are incorporated herein by reference in their entirety. For example, the linker may be a C₁-C₁₀ straight chain alkyl, C₁-C₁₀ straight chain O-alkyl, C₁-C₁₀ straight chain substituted alkyl, C₁-C₁₀ straight chain substituted O-alkyl, C₄-C₁₃ branched chain alkyl, C₄-C₁₃ branched chain O-alkyl, C₂-C₁₂ straight chain alkenyl, C₂-C₁₂ straight chain O-alkenyl, C₃-C₁₂ straight chain substituted alkenyl, C₃-C₁₂ straight chain substituted O-alkenyl, polyethylene glycol, polylactic acid, polyglycolic acid, poly(lactide-co-glycolide), polycarprolactone, polycyanoacrylate, ketone, aryl, heterocyclic, succinic ester, amino acid, aromatic group, ether, crown ether, urea, thiourea, amide, purine, pyrimidine, bypiridine, indole derivative acting as a cross linker, chelator, aldehyde, ketone, bisamine, bis alcohol, heterocyclic ring structure, azirine, disulfide, thioether, hydrazone and combinations thereof. For example, the linker can be a C₃ straight chain alkyl or a ketone. The alkyl chain of the linker can be substituted with one or more substituents or heteroatoms. In some embodiments the linker contains one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid.

In some embodiments, the linker may be cleavable and is cleaved to release the active agent. In one embodiment, the linker may be cleaved by an enzyme. As a non-limiting example, the linker may be a polypeptide moiety, e.g. AA in WO2010093395 to Govindan, the contents of which are incorporated herein by reference in their entirety, that is cleavable by intracellular peptidase. Govindan teaches AA in the linker may be a di, tri, or tetrapeptide such as Ala-Leu, Leu-Ala-Leu, and Ala-Leu-Ala-Leu. In another example, the cleavable linker may be a branched peptide. The branched peptide linker may comprise two or more amino acid moieties that provide an enzyme cleavage site. Any branched peptide linker disclosed in WO1998019705 to Dubowchik, the contents of which are incorporated herein by reference in their entirety, may be used as a linker in the conjugate of the present invention. As another example, the linker may comprise a lysosomally cleavable polypeptide disclosed in U.S. Pat. No. 8,877,901 to Govindan et al., the contents of which are incorporated herein by reference in their entirety. As another example, the linker may comprise a protein peptide sequence which is selectively enzymatically cleavable by tumor associated proteases, such as any Y and Z structures disclosed in U.S. Pat. No. 6,214,345 to Firestone et al., the contents of which are incorporated herein by reference in their entirety.

In one embodiment, the cleaving of the linker is non-enzymatic. Any linker disclosed in US 20110053848 to Cleemann et al., the contents of which are incorporated herein by reference in their entirety, may be used. For example, the linker may be a non-biologically active linker represented by formula (I).

In one embodiment, the linker may be a beta-glucuronide linker disclosed in US 20140031535 to Jeffrey, the contents of which are incorporated herein by reference in their entirety. In another embodiment, the linker may be a self-stabilizing linker such as a succinimide ring, a maleimide ring, a hydrolyzed succinimide ring or a hydrolyzed maleimide ring, disclosed in US20130309256 to Lyon et al., the contents of which are incorporated herein by reference in their entirety. In another embodiment, the linker may be a human serum albumin (HAS) linker disclosed in US 20120003221 to McDonagh et al., the contents of which are incorporated herein by reference in their entirety. In another embodiment, the linker may comprise a fullerene, e.g., C₆₀, as disclosed in US 20040241173 to Wilson et al., the contents of which are incorporated herein by reference in their entirety. In another embodiment, the linker may be a recombinant albumin fused with polycysteine peptide as disclosed in U.S. Pat. No. 8,541,378 to Ahn et al., the contents of which are incorporated herein by reference in their entirety. In another embodiment, the linker comprises a heterocycle ring. For example, the linker may be any heterocyclic 1,3-substituted five- or six-member ring, such as thiazolidine, disclosed in US 20130309257 to Giulio, the contents of which are incorporated herein by reference in their entirety.

In one embodiment, the linker may be any cleavable linker disclosed in US 2015/0023912 to Kratz et al., the contents of which are incorporated herein by reference in their entirety. For example, the cleavable linker may be cleaved by an enzyme such as but not limited to proteases and peptidases, e.g., matrix metalloproteases (MMP), cysteine proteases, serine proteases and plasmin activators, which are formed or activated in intensified manner in diseases such as rheumatoid arthritis or cancer. Non-limiting examples of proteases include MMP-2, MMP-3 and MMP-9, cathepsin B, H, L and D, plasmin, urokinase, and prostate-specific antigen (PSA). The cleavable linker may contain a peptide bond such as but not limited to Arg-Arg, Phe-Arg, Phe-Cit, Ile-Pro, Lys, Lys-Lys, Arg-Lys, Ala-Leu-Ala-Leu, Phe-Lys, Phe-Lys-Ala, Val-Cit, Val-Arg, Ala-Phe-Lys, D-Ala-Phe-Lys, Met, Met-Met, Phe-Met, Tyr-Met, Ala-Met, Ala-Phe-Met, Phe-Ala-Met, Ala-Tyr-Met, Phe-Tyr-Met, Ser-Ser-Tyr-Tyr-Ser-Arg, Ser-Ser-Tyr-Tyr-Ser-Leu, Arg-Ser-Ser-Tyr-Tyr-Ser-Leu, Phe-Pro-Lys-Phe-Phe-Ser-Arg-Gln, Lys-Pro-lle-Glu-Phe-Nph-Arg-Leu, Gly-Pro-Leu-Gly-Ile-Ala-Gly-Gln, Gly-Pro-Leu-Gly-Ile-Ala-Gly-Gln, Gly-Pro-Gln-Gly-Ile-Trp-Gly-Gln, Gly-Phe-Leu-Gly. The enzymatically cleavable linker may contain a self-immolative linker such as a self-immolative p-aminobenzyloxycarbonyl (PABC) linker or a N-methyl- or symmetric N,N-dimethylethylene linker.

Linkers may be cleaved in a variety of ways to release the active therapeutic agent. These include acidic hydrolysis, enzymatic hydrolysis and reductive processes. Some chemical bonds such as hydrazone, ester and amide bonds are sensitive to acidic pH values, for example, of the intracellular environment of tumor cells. At acidic pH, hydrogen ions catalyze the hydrolysis of these bonds which in turn releases the active agent.

An non-limiting example of a Pt(IV)M compound comprising cabazitaxel as an active agent. The Pt(IV)M compound may have a Formula A3 of:

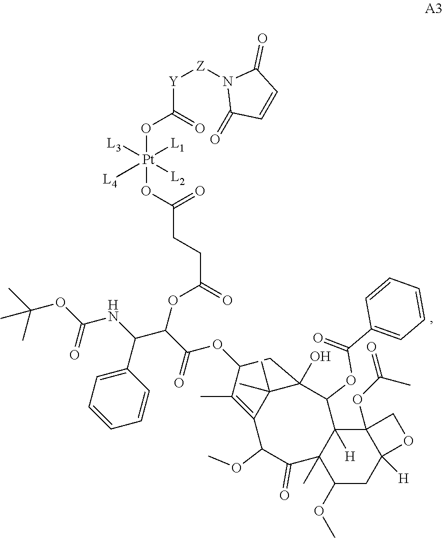

wherein Y is selected from NH, alkyl and aryl;
Z is alternatively absent or alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl; and
L₁, L₂, L₃ and L₄ are independently any suitable ligand for Pt.

In some embodiments, Y together with Z and the maleimide may form a group selected from the group consisting of:

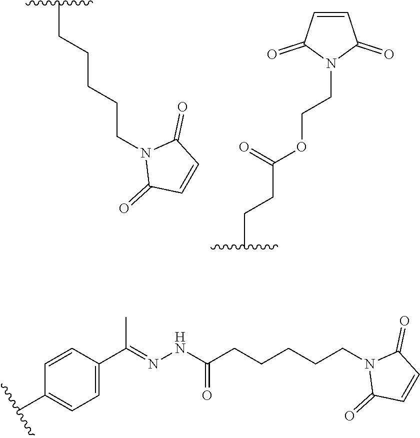 [SMILES: *CCCCCN1C(=O)C=CC1=O ; *CCC(=O)OCCN1C(=O)C=CC1=O ; *c1ccc(/C(C)=N/NC(=O)CCCCCN2C(=O)C=CC2=O)cc1]

As a non-limiting example, L₁ and L₂ may be Cl; L₃ and L₄ may be NH₃. The Pt(IV)M compound may have a formula of

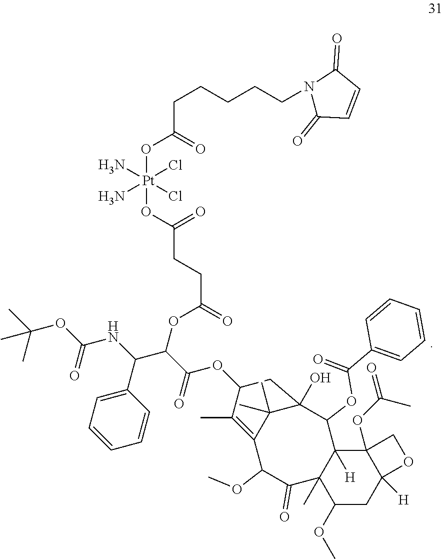

As described herein, some compounds of the present teachings may be provided as a salt comprising a charged platinum complex and a counter ion, including a pharmaceutically acceptable counter ion. The counter ion may be a weak or non-nucleophilic stabilizing ion, having a charge of (−1), (−2), (−3), (+1), (+2), (+3), etc. In some embodiments, the counter ion has a charge of (−1). In other embodiments, the counter ion has a charge of (−2). In some embodiments, the counter ion has a charge of (+1). In other embodiments, the counter ion has a charge of (+2).

The present teachings further comprise compositions (including pharmaceutical compositions) each comprising one or more of the compounds as described herein, and at least one pharmaceutically acceptable excipient.

II. Combinations with Other Active Agents

The Pt(IV)M compounds of the present application may be combined with at least one other active agent to form a composition. The at least one active agent in the Pt(IV)M compound may be a therapeutic, prophylactic, diagnostic, or nutritional agent. It may be a small molecule, protein, peptide, lipid, glycolipid, glycoprotein, lipoprotein, carbohydrate, sugar, or nucleic acid.

In some embodiments, the at least one other active agent may be any therapeutically active agent disclosed in US 2014/0378427 to Bilodeau et al, the contents of which are incorporated herein by reference in their entirety. For example, the at least one other active agent may be agents capable of preventing the establishment or growth of a tumor or infection, treating or preventing the establishment or growth of a bacterial infection, or an anti-cancer drug taught in US 2014/0378427 to Bilodeau.

In some embodiments, the at least one other active agent may be any chemotherapeutic agent anti-infective agent disclosed in US 2014/0187501 to Bilodeau et al, the contents of which are incorporated herein by reference in their entirety. In some embodiments, the at least one other active agent is an antigen or adjuvant, radioactive or imaging agent (e.g., a fluorescent moiety) or polynucleotide. In some embodiments the active agent is an organometallic compound. For example, the active agent may be Cabazitaxel, Gemcitabine, Docetaxel, Paclitaxel, Etoposide, 5-FU, Leucovorin, Irinotecan, Cetuximab, Capecitabine, Methotrexate, Vinblastine, or Doxorubicin.

In some other embodiments, the at least one other active agent may be a cytostatic agent, a cytokine, an immunosuppressive agent, a virostatic agent, an antirheumatic agent, an analgesic, an anti-inflammatory agent, an antibiotic, an antimycotic agent, a signal transduction inhibitor, an angiogenesis inhibitor, and a protease inhibitor.

In some other embodiments, the at least one other active agent is an enzyme inhibitor. The enzyme may be an oxidoreductase, a transferase, a hydrolase, a lyase, etc. For example, the at least one other active agent may be a poly ADP ribose polymerase (PARP) inhibitor such as iniparib (BSI 201), talazoparib (BMN-673), olaparib (AZD-2281), rucaparib (AG014699, PF-01367338), veliparib (ABT-888), CEP 9722, MK 4827, BGB-290, or 3-aminobenzamide.

In another embodiment, the at least one other active agent may be an albumin-binding agent. Any therapeutically effective substance disclosed in U.S. Pat. No. 8,183,581 to Kratz et al., the contents of which are incorporated herein by reference in their entirety, comprising a protein-binding molecular residue linked to an active compound, may be used.

In another embodiment, the at least one other active agent may be any albumin-based drug delivery system disclosed in US 2015/0023912 to Kratz et al., the contents of which are incorporated herein by reference in their entirety, such as 6-maleimidocaproyl(hydrazone) derivative of doxorubicin (DOXO-EMCH), an albumin-binding prodrug selected from the methotrexate derivative EMC-D-Ala-Phe-Lys-Lys(γ-MTX)-OH, wherein EMC=6-maleimidocaproic acid, (AW054), an albumin-drug nanoparticle, selected from Nab®-paclitaxel (ABI-007, Abraxane™), Nab®-docetaxel (ABI-008) or Nab®-rapamycin (ABI-010), an albumin-binding antibody construct selected from camelid anti-HSA trivalent nanobodies, or an albumin fusion protein with interferons or interleukins selected from albinterferon alfa-2b or albuleukin.

In another embodiment, the at least one other active agent may a prodrug consisting of at least two different therapeutically and/or diagnostically active compounds bound by cleavable linkers, a spacer molecule, and of at least one protein-binding molecule capable of binding to a carrier molecule as disclosed in U.S. Pat. No. 8,153,581 and U.S. Pat. No. 8,664,181 to Kratz et al., the contents of each of which are incorporated herein by reference in their entirety. In some embodiments, the therapeutically active compound is a cytostatic agent, a cytokine, an immunosuppressive agent, a virostatic agent, an antirheumatic agent, an analgesic, an antiinflammatory agent, an antibiotic, an antimycotic agent, a signal transduction inhibitor, an angiogenesis inhibitor or a protease inhibitor. In some embodiments, a diagnostically active compound may include a labelled therapeutically active compound or one or more fluorescent compound(s) and/or one or more contrast agents in the near IR range. In some embodiments, a protein binding-molecule is preferably a maleimide group, a haloacetamide group, a haloacetate group, a pyridyldithio group, an N-hydroxysuccinimide ester group or an isothiocyanate group. In some embodiments, it can also be a disulphide group, a vinylcarbonyl group, an aziridine group or an acetylene group.

In another embodiment, the at least one other active agent may be a carrier-drug conjugate comprising a drug, a spacer molecule, and a thiol-binding group as disclosed in U.S. Pat. No. 7,445,764 to Kratz et al., the contents of which are incorporated herein by reference in their entirety. Kratz discloses that a carrier contains a polypeptide sequence having one or a plurality of cysteine groups. The drug may include a pharmaceutically and/or diagnostically active substance that brings about a pharmacological effect either by itself or after its conversion by metabolism in the organism, and thus also includes the derivatives resulting from these conversions. The diagnostically active substance may be detected, preferably also quantified, in the organism or parts thereof, through suitable chemical and/or physical measurement methods. The thiol-binding group may contain a maleinimide group, a haloacetamide group, a haloacetate group, a pyridyldithio group, a vinylcarbonyl group, an aziridine group, a disulfide group or an acetylene group, which are substituted if appropriate.

In another embodiment, the at least one other active agent may be a dual acting prodrug comprising at least a first pharmaceutically and/or diagnostically active compound, at least a second pharmaceutically and/or diagnostically active compound, two or more cleavable linkers, and a protein binding moiety as disclosed in US 20140205539 to Kratz et al., the contents of which are incorporated herein by reference in their entirety. The expression “pharmaceutically active compound” means any compound which brings about a pharmacological effect either by itself or after its conversion in the organism in question, and thus also includes the derivatives from these conversions. The expression “diagnostically active compound” used herein is not specifically restricted and includes any compound which can be detected and preferably quantified, in an organism or parts thereof, such as for example cells and/or fluids, such as for example the serum, through suitable chemical and/or physical measurement methods.

In another embodiment, the at least one other active agent is a prodrug comprising at least one pharmaceutically and/or diagnostically active compound bound by a cleavable linker, a receptor and/or targeting moiety and a protein binding moiety which is capable of binding to a carrier molecule as disclosed in US 20100111866 to Kratz, et al., the contents of which are incorporated herein by reference in their entirety. The term “receptor and/or antigen targeting moiety”, as used herein, is not specifically restricted and means that it preferably interacts with the receptor or antigen by physically and/or chemically binding to it as a ligand. In some embodiments, preferred receptors and/or antigens that are targeted by the ligand of the prodrug may be upregulated or exclusively expressed in tissues associated with a disease. In some embodiments, preferred examples of such receptors are receptors of growth factors, of vitamins, of cytokines, of hormones, of peptides, of plasma proteins, of the endothelium, or G-protein coupled receptors. In some embodiments, especially preferred examples of antigens are those associated with cancer and inflammatory diseases.

In another embodiment, the at least one other active drug may be a prodrug comprising at least one cytostatic agent, which after administration may covalently bind to circulating albumin and are cleaved by prostate-specific antigen (PSA) in tumor tissue to release the active ingredient for use in the treatment of cancer as disclosed in U.S. Pat. No. 8,642,555 and US 20080161245 to Kratz et al., the contents of each of which are incorporated herein by reference in their entirety. In some embodiments, cytostatic agents are preferably N-nitrosoureas, anthracyclines, low-molecular anthracycline peptide derivatives, alkylating agents, antimetabolites, folic acid antagonists, camptothecins, Vinca alkaloids, taxanes, calicheamicins, maytansinoids, auristatins, epothilones, bleomycin, dactinomycin, plicamycin, mitomycin C and cis-configured platinum(II) complexes. In some embodiments, the prostate-specific antigen (PSA) represents a molecular target for selectively releasing an anticancer agent from a prodrug formulation.

In another embodiment, the at least one other active agent may comprise transport molecule binding ligand compounds, a therapeutically and/or diagnostically active substance and a carrier molecule-affinitive substance as disclosed in U.S. Pat. No. 7,902,144 to Kratz et al., the contents of which are incorporated herein by reference in their entirety. In some embodiments, the therapeutically and/or diagnostically active substance is a cytostatic agent, a cytokine, an immunosuppressant, an antirheumatic, an antiinflammatory, an antibiotic; an analgesic, a virostatic or an antimycotic. In some embodiments, the carrier molecule comprises both natural and synthetic molecules that are suitable for transporting ligand compounds in body fluids such as blood serum.

In another embodiment, the at least one other active agent may be chemotherapeutic conjugates comprising a doxorubicin-peptide derivative that may bind covalently to circulating albumin after intravenous administration and are cleaved by the matrix metalloproteinases 2 and/or 9 (MMP-2 and/or MMP-9) in the tumor tissue as disclosed in U.S. Pat. No. 7,803,903 to Kratz et al., the contents of which are incorporated herein by reference in their entirety. In some embodiments, the doxorubicin-peptide derivatives comprise doxorubicin, a peptide spacer, and a heterobifunctional cross-linker containing a protein-binding group. In some embodiments, the peptides are enzymatically cleaved by matrix metalloproteinases 2 and/or 9.

In another embodiment, the at least one other active agent may be platinum complexes comprising cyclobutane-1,1-dicarboxylate ligands which may further contain a protein-binding group as disclosed in U.S. Pat. No. 7,141,691 to Kratz et al., the contents of which are incorporated herein by reference in their entirety. In some embodiments, the platinum complexes may have fewer undesired side-effects and a higher efficacy towards tumor tissue.

In another embodiment, the at least one other active agent may be drug-conjugates comprising transferrin, albumin and polyethylene glycol and at least one cytostatic compound that has a tumor-inhibiting effectiveness as disclosed in U.S. Pat. No. 6,709,679 to Kratz et al., the contents of which are incorporated herein by reference in their entirety. In some embodiments, the cytostatic compounds comprise anthracyclines, doxorubicin, daunorubicin, epirubicin, idarubicin and mitoxandrone, the alkylates, chloroambucil and melphalan, the antimetabolites, methotrexate, 5-fluorouracyl, 5′-desoxy-5-fluorouridine and thioguanine, the taxoides, paclitaxel and docetaxel, the camptothecins, topotecan and 9-aminocamptothecin, the podophyllotoxin derivatives, etoposide, teniposide and mitopodoside, the vinca alkaloids, vinblastine, vincristine, vindesine and vinorelbine.

In another embodiment, the at least one other active agent may be a pharmaceutical composition comprising at least two different albumin-based drug delivery systems as disclosed in US 20150023912 to Kratz et al., the contents of which are incorporated herein by reference in their entirety. In some embodiments, at least one of said at least two different albumin-based drug delivery systems is selected from albumin-binding prodrugs, albumin drug conjugates, albumin peptide conjugates, albumin fusion proteins, albumin-binding peptide conjugates, albumin drug nanoparticles, and albumin-based antibody constructs.

In another embodiment, the at least one other active agent may be a pharmaceutical composition or kit comprising a combination of at least one first drug and at least one protein-binding prodrug, wherein the protein binding prodrug comprises a protein-binding group, a second drug, and a linker that can be cleaved hydrolytically, enzymatically, or in a pH-dependent manner in the body, as disclosed in US 20130040905 to Kratz et al., the contents of which are incorporated herein by reference in their entirety. The term “first drug” and “second drug”, as used herein, is not specifically restricted and means that first drug and the second drug contained in the prodrug are independently selected from the group consisting of a cytostatic agent, a cytokine, an immunosuppressant, an antirheumatic, an antiphlogistic, an antibiotic, an analgesic, a virostatic, an antimycotic agent, a transcription factor inhibitor, a cell cycle modulator, an MDR modulator, a proteasome or protease inhibitor, an apoptosis modulator, a histone deacetylase inhibitor, an enzyme inhibitor, an angiogenesis inhibitor, a hormone or hormone derivative, a radioactive substance, a light emitting substance, or a light absorbing substance. The term “protein-binding prodrug”, as used herein, is not specifically restricted and means that the protein-binding prodrug comprises a protein-binding group, a second drug, and a linker that can be cleaved hydrolytically, enzymatically, or in a pH-dependent manner, and wherein the at least one first drug and the second drug contained in the prodrug are the same or different.

In another embodiment, the at least one other active agent may be a drug polymer conjugate comprising a pharmaceutically and/or diagnostically active compound bound to a dendritic polyglycerol core having a polyethylene glycol shell as disclosed in US 20110117009 to Kratz et al., the contents of which are incorporated herein by reference in their entirety. The term “drug polymer conjugate”, as used herein, is not specifically restricted and includes any compound that is bound to a dendritic polyglycerol. The term “dendritic polyglycerol”, as used herein, is not specifically restricted and includes any substance which contains at least two glycerol units in its molecule and wherein said molecule is characterized by a branched structure. In some embodiments, the dendritic core of the conjugate allows the binding of a polyethylene glycol shell and of one or more pharmaceutically and/or diagnostically active substances to yield a new class of highly versatile prodrugs.

In another embodiment, the at least one other active agent may be methotrexate derivatives comprising a protein-binding group that can be enzymatically cleaved in the body such that the active substance or a low-molecular active substance derivative is released, as disclosed in US 20100041615 to Kratz et al., the contents of which are incorporated herein by reference in their entirety. In some embodiments, methotrexate derivatives are composed of an antitumor or antirheumatic methotrexate component, a spacer molecule, a peptide chain and a heterobifunctional crosslinker that may be administered parenterally, preferably intravenously.

In another embodiment, the at least one other active agent may be chemoimmunoconjugates comprising conjugates of transferrin, albumin and polyethylene glycol consisting of native or thiolated transferrin or albumin or of polyethylene glycol with at least one thiol-, hydroxyl- or amine-group and cytostatic compounds derived through maleinimide or N-hydroxysuccinimide ester-compounds as disclosed in EP 1447099 and WO 1998010794 to Kratz, et al., the contents of each of which are incorporated herein by reference in their entirety. In some embodiments, cytostatic compounds include doxorubicene, daunorubicene, epirubicene, idarubicene, mitoxandrone, chlorambucil, melphalan, 5-fluorouracyl, 5′-desoxy-5-fluorouridene, thioguanine, methotrexate, paclitaxel, docetaxel, topotecan, 9-aminocamptothecene, etoposide, teniposide, mitopodoside, vinblastine, vincristine, vindesine, vinorelbin.

In another embodiment, the at least one other active agent may be camptothecin peptide derivatives comprising a protein-binding group, which may be enzymatically cleaved in vivo and release the active ingredient or a low-molecular weight active ingredient derivative as disclosed in WO 2006092230 to Kratz et al., the contents of which are incorporated herein by reference in their entirety.

The Pt(IV)M compound and the at least one other active agent may be administered semitaneously or sequentially. They may be present as a mixture for simultaneous administration, or may each be present in separate containers for sequential administration.

The term “simultaneous administration”, as used herein, is not specifically restricted and means that the Pt(IV)M compound and the at least one other active agent are substantially administered at the same time, e.g. as a mixture or in immediate subsequent sequence.

The term “sequential administration”, as used herein, is not specifically restricted and means that the Pt(IV)M compound and the at least one other active agent are not administered at the same time but one after the other, or in groups, with a specific time interval between administrations. The time interval may be the same or different between the respective administrations of Pt(IV)M compound and the at least one other active agent and may be selected, for example, from the range of 2 minutes to 96 hours, 1 to 7 days or one, two or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

The molar ratio of the Pt(IV)M compound and the at least one other active agent is not particularly restricted. For example, when the Pt(IV)M compound and one other active agent are combined in a composition, the molar ratio of them may be in the range of 1:500 to 500:1, or of 1:100 to 100:1, or of 1:50 to 50:1, or of 1:20 to 20:1, or of 1:5 to 5:1, or 1:1. Similar molar ratios apply when the Pt(IV)M compound and two or more other active agent are combined in a composition. The Pt(IV)M compound may comprise a predetermined molar weight percentage from about 1% to 10%, or about 10% to about 20%, or about 20% to about 30%, or about 30% to 40%, or about 40% to 50%, or about 50% to 60%, or about 60% to 70%, or about 70% to 80%, or about 80% to 90%, or about 90% to 99% of the composition.

### III. Formulation, Delivery, Administration, and Dosing

In some embodiments, compositions are administered to humans, human patients or subjects. For the purposes of the present disclosure, the phrase “active ingredient” generally refers to the Pt(IV)M compounds to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The Pt(IV)M compounds of the present invention can be formulated using one or more excipients to: (1) increase stability; (2) permit the sustained or delayed release (e.g., from a depot formulation of the Pt(IV)M compounds); (3) alter the biodistribution (e.g., target the Pt(IV)M compounds to specific tissues or cell types); (4) alter the release profile of the Pt(IV)M compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the Pt(IV)M compounds.

In some embodiments, the pH value of the pharmaceutical composition is between about 4 to about 7, between 4 and 6, between 4 and 5, about 4, about 5, about 6 or about 7.

Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Administration

The Pt(IV)M compounds of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier.

### Dosing

The present invention provides methods comprising administering Pt(IV)M compounds to a subject in need thereof. Pt(IV)M compounds as described herein may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose. In one embodiment, the Pt(IV)M compounds of the present invention are administered to a subject in split doses. The Pt(IV)M compounds may be formulated in buffer only or in a formulation described herein.

Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous).

Liquid Dosage Forms

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art including, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions may be mixed with solubilizing agents such as CREMOPHOR®, alcohols, oils, modified oils, glycols, polysorbates, cyclodextrins, polymers, and/or combinations thereof.

Injectable

Injectable preparations, for example, sterile injectable aqueous or oleaginous suspensions may be formulated according to the known art and may include suitable dispersing agents, wetting agents, and/or suspending agents. Sterile injectable preparations may be sterile injectable solutions, suspensions, and/or emulsions in nontoxic parenterally acceptable diluents and/or solvents, for example, a solution in 1,3-butanediol. Among the acceptable vehicles and solvents that may be employed include, but are not limited to, water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it may be desirable to slow the absorption of the active ingredient from subcutaneous or intramuscular injection. This may be accomplished by the use of a liquid suspension of crystalline or amorphous material with poor water solubility. The rate of absorption of the Pt(IV)M compounds then depends upon its rate of dissolution which, in turn, may depend upon crystal size and crystalline form. Alternatively, delayed absorption of a parenterally administered Pt(IV)M compound may be accomplished by dissolving or suspending the monomalimide in an oil vehicle. Injectable depot forms are made by forming microencapsule matrices of the Pt(IV)M compounds in biodegradable polymers such as polylactide-polyglycolide. Depending upon the ratio of Pt(IV)M compounds to polymer and the nature of the particular polymer employed, the rate of Pt(IV)M compound release can be controlled. Examples of other biodegradable polymers include, but are not limited to, poly(orthoesters) and poly(anhydrides). Depot injectable formulations may be prepared by entrapping the Pt(IV)M compounds in liposomes or microemulsions which are compatible with body tissues.

Pulmonary

Formulations described herein as being useful for pulmonary delivery may also be used for intranasal delivery of a pharmaceutical composition. Another formulation suitable for intranasal administration may be a coarse powder comprising the active ingredient and having an average particle from about 0.2 um to 500 um. Such a formulation may be administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain about 0.1% to 20% (w/w) active ingredient, where the balance may comprise an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### IV. Pharmaceutical Compositions and Methods of Use

Embodiments of the present teachings also relate to treating a hyperproliferative disorder, cancer and/or a tumor according to any of the techniques and compositions and combinations of compositions described herein.

In various embodiments, methods for treating a subject having a cancer are provided, wherein the method comprises administering a therapeutically-effective amount of a compound and/or composition, as described herein, to a subject having a cancer, suspected of having cancer, or having a predisposition to a cancer. According to the present invention, cancer embraces any disease or malady characterized by uncontrolled cell proliferation, e.g., hyperproliferation. Cancers may be characterized by tumors, e.g., solid tumors or any neoplasm.

In some embodiments, the subject may be otherwise free of indications for treatment with the compound. In some embodiments, methods include use of cancer cells, including but not limited to mammalian cancer cells. In some instances, the mammalian cancer cells are human cancer cells.

In some embodiments, compounds and/or compositions of the present teachings have been found to inhibit cancer and/or tumor growth. They may also reduce cell proliferation, invasiveness, and/or metastasis, thereby rendering them useful for the treatment of a cancer.

In some embodiments, compounds and/or compositions of the present teachings may be used to prevent the growth of a tumor or cancer, and/or to prevent the metastasis of a tumor or cancer. In some embodiments, compounds and/or compositions of the present teachings may be used to shrink or destroy a cancer.

In some embodiments, a compound and/or composition provided herein is useful for inhibiting proliferation of a cancer cell. In some embodiments a compound and/or composition provided herein is useful for inhibiting cellular proliferation, e.g., inhibiting the rate of cellular proliferation, preventing cellular proliferation, and/or inducing cell death. In general, a compound and/or composition as described herein can inhibit cellular proliferation of a cancer cell or both inhibiting proliferation and/or inducing cell death of a cancer cell.

The cancers treatable by methods of the present teachings generally occur in mammals. Mammals include, for example, humans, non-human primates, dogs, cats, rats, mice, rabbits, ferrets, guinea pigs horses, pigs, sheep, goats, and cattle. In various embodiments, the cancer is lung cancer, e.g., small cell lung cancer, non-small cell lung cancer, squamous cell lung cancer, breast cancer, e.g., mutant BRCA1 and/or mutant BRCA2 breast cancer, non-BRCA-associated breast cancer, colorectal cancer, colon cancer, ovarian cancer, pancreatic cancer-bladder cancer, prostate cancer, cervical cancer, renal cancer, leukemia, central nervous system cancers, myeloma, melanoma, mesothelioma, stomach cancer, rectal cancer, cancer of the large intestine, cancer of the small intestine, esophageal cancer, uterine cancer, head and neck cancer, endometrial cancer, eye cancer, thyroid cancer, testicular cancer, bile duct cancer, liver cancer, kidney cancer, pituitary cancer, lymphoma, brain cancer, glioma, glioblastoma multiforme, meningioma, medulloblastoma, astrocytoma, neuroblastoma, basal cell carcinoma of the skin, sarcoma, synovial sarcoma, rhabdomyosarcoma, leiomyosarcoma, chondrosarcoma, and fibrosarcoma. In some embodiments, the cancer is lung cancer. In certain embodiments, the cancer is human lung carcinoma, ovarian cancer, pancreatic cancer or colorectal cancer.

In some embodiments, compounds and/or compositions of the present teachings may be administered to the cancer cells having BRCA1 mutations, BRCA2 mutations, ERCC1 mutations, ERCC2 mutations, mutations in the fanconi anemia genes, MLH1, MSH2, PTEN, Mutations in genes that code for proteins involved in DNA repair, mutations in genes that code for proteins involved in non-homologous DNA repair, mutations in genes that code for proteins involved in nucleotide excision repair, mutations in genes that code for proteins involved in DNA mismatch repair, genetic tests that identify tumors that have a defect in DNA repair, changes in the expression of genes involved in DNA repair such as ERCC1, ERCC2, and so on. The mutations may be germline or somatic.

In another aspect, compounds and/or compositions of the present teachings may be administered to cells with increased albumin uptake, for example, but not limited to, cells with mutations that increase micropinocytosis, cells with mitogen activated kinase pathway mutations, cells with KRAS mutations, cells with BRAF mutations, cells with RAC mutations, cells with RAS overexpression, cells with RAC1 activation, or cells with CDC42 activation.

In some embodiments, cells with increased albumin update may be identified with imaging techniques. For example, a contrast agent is administered to a patient and the level of accumulation of the contrast agent at a tumor site is measured with an imaging technique. The imaging technique may be ultrasound, X-ray, single-photon emission tomography/computed tomography (SPECT/CT), positron emission tomography/computed tomography (PET/CT), magnetic resonance imaging (MRI), computed tomography (CT), single-photon emission tomography (SPECT), fluorescence tomography, and fluorescence spectroscopy.

In yet another aspect, compounds and/or composition of the present teachings may be administered to tumors with a high level of enhanced permeability and retention (EPR) effect. In some embodiments, tumors with a high level of enhanced permeability and retention effect may be identified with imaging techniques. As a non-limited example, iron oxide nanoparticle magnetic resonance imaging may be administered to a patient and EPR effects are measured.

In some embodiments, compounds and/or composition of the present teachings may be administered to a subject selected with the method disclosed in WO2015017506, the contents of which are incorporated herein by reference in their entirety, the method comprising:

a) administering a contrast agent to the subject;
(b) measuring the level of accumulation of the contrast agent at at least one intended site of treatment; and
(c) selecting the subject based on the level of the accumulation of the contrast agent;
wherein the intended site of treatment is a tumor.

In some embodiments, compounds and/or compositions of the present teachings may be used to treat a viral disease, autoimmune disease, acute or chronic inflammatory disease, and a disease caused by bacteria, fungi, or other micro-organisms.

V. Combination with Other Therapies

In some embodiments, the Pt(IV)M compounds and/or compositions of the present application may be part of a combination therapy. For example, they may be combined with another therapy, such as chemotherapy, surgical treatment, radiation therapy, immunotherapy, and/or with any other antineoplastic treatment method.

As used herein, the term “combination therapy” means a therapy strategy that embraces the administration of therapeutical compositions of the present invention (e.g., conjugates comprising one or more neoantigens) and one or more additional therapeutic agents as part of a specific treatment regimen intended to provide a beneficial (additive or synergistic) effect from the co-action of these therapeutic agents. Administration of these therapeutic agents in combination may be carried out over a defined time period (usually minutes, hours, days, or weeks depending upon the combination selected). In combination therapy, combined therapeutic agent may be adminstered in a sequential manner, or by substantially simultaneous administration.

As used herein, antineoplastic or chemotherapeutic agent refers to agents which preferentially kill neoplastic cells or disrupt the cell cycle of rapidly proliferating cells, used therapeutically to prevent or reduce the growth of neoplastic cells. As used herein, chemotherapy includes treatment with a single chemotherapeutic agent or with a combination of agents.

As used herein, the term “immunotherapy” refers to any therapy that can provoke and/or enhance an immune response to destroy tumor cells in a subject.

In some embodiments, the Pt(IV)M compounds and/or compositions of the present application may be combined with active immunotherapy and/or adoptive immunotherapy to prevent/treat a disease such as cancer. In an active immunotherapy, tumor antigen specific T cells are primed and amplified in vivo. For an adoptive immunotherapy, T cells are isolated from a subject to be treated and may be primed and amplified ex vivo prior to their infusion. Adoptive immunotherapy is a procedure whereby an individual's own T cells are expanded ex vivo and re-infused back into the body. Both active and adoptive immunotherapy can be used as therapeutic strategies for treatment of cancer in combination with the Pt(IV)M compounds and/or compositions of the present application.

In some embodiments, the Pt(IV)M compounds and/or compositions of the present application may be combined with tumor infiltrating lymphocytes (TIL). TILs are a class of lymphocytes derived from primary or metastatic tumor tissue fragments, regionally tumor-draining lymph nodes or malignant ascites, which can be expanded in vitro in IL-2-supplemented media and enriched predominantly in CD8+ cytotoxic T lymphocytes (CTLs) in order to eradicate autologous tumor antigens in a MHC-restricted pattern.

In some embodiments, the Pt(IV)M compounds and/or compositions of the present application may be combined with Chimeric Antigen Receptor (CAR) T cell immunotherapy. CAR-engineered T cells combined TAA-recognized single-chain antibody with the activation motif of T cells, freeing antigen recognition from MHC restriction and thus breaking one of the barriers to more widespread application of ACI. It means combining the high affinity of antibody to TAA with the killing mechanism of T cells. It had been bolstered that CAR-engineered T cells exhibited antitumor function to prostate cancer and other advanced malignancies.

In some embodiments, the Pt(IV)M compounds and/or compositions of the present application may be combined with cancer vaccines and/or complementary immunotherapeutics such as immune checkpoint-blockade inhibitors. As used herein, the term “vaccine” refers to a composition for generating immunity for the prophylaxis and/or treatment of diseases.

### VI. Kits and Devices

The invention provides a variety of kits and devices for conveniently and/or effectively carrying out methods of the present invention. Typically kits will comprise sufficient amounts and/or numbers of components to allow a user to perform multiple treatments of a subject(s) and/or to perform multiple experiments.

In one embodiment, the present invention provides kits for inhibiting tumor cell growth in vitro or in vivo, comprising a Pt(IV)M compound of the present invention or a combination of Pt(IV)M compounds of the present invention, optionally in combination with at least one other active agent.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of Pt(IV)M compounds in the buffer solution over a period of time and/or under a variety of conditions.

The present invention provides for devices which may incorporate Pt(IV)M compounds of the present invention. These devices contain in a stable formulation available to be immediately delivered to a subject in need thereof, such as a human patient. In some embodiments, the subject has cancer.

Non-limiting examples of the devices include a pump, a catheter, a needle, a transdermal patch, a pressurized olfactory delivery device, iontophoresis devices, multi-layered microfluidic devices. The devices may be employed to deliver Pt(IV)M compounds of the present invention according to single, multi- or split-dosing regiments. The devices may be employed to deliver Pt(IV)M compounds of the present invention across biological tissue, intradermal, subcutaneously, or intramuscularly. More examples of devices suitable for delivering Pt(IV)M compounds include but not limited to a medical device for intravesical drug delivery disclosed in International Publication WO 2014036555, a glass bottle made of type I glass disclosed in US Publication No. 20080108697, a drug-eluting device comprising a film made of a degrable polymer and an active agent as disclosed in US Publication No. 20140308336, an infusion device having an injection micropump, or a container containing a pharmaceutically stable preparation of an active agent as disclosed in U.S. Pat. No. 5,716,988, an implantable device comprising a reservoir and a channeled member in fluid communication with the reservoir the as disclosed in International Publication WO 2015023557, a hollow-fibre-based biocompatible drug delivery device with one or more layers as disclosed in US Publication No. 20090220612, an implantable device for drug delivery including an elongated, flexible device having a housing defining a reservoir that contains a drug in solid or semi-solid form as disclosed in International Publication WO 2013170069, a bioresorbable implant device disclosed in U.S. Pat. No. 7,326,421, contents of each of which are incorporated herein by reference in their entirety.

It will be appreciated that the following examples are intended to illustrate but not to limit the present invention. Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

### EXAMPLES

### Example A: General Scheme for the Synthesis of

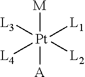

### Compounds

The Pt(IV)M compounds comprising at least one active agent may, in some embodiments, be prepared by: a) providing an active agent having a hydroxyl group and reacting it with succinic anhydride to form the conjugate of active agent-succinate, b) reacting a platinum complex with a coupling reagent and the active agent-succinate to form the active agent-linker-platinum complex conjugate.

When a linker is employed to attach the active agent through environmentally cleavable bonds, any of a variety of methods can be used to associate the linker with the active agent including, but not limited to, passive adsorption, e.g., via electrostatic interactions), multivalent chelation, high affinity non-covalent binding between members of a specific binding pair, covalent bond formation, etc. In some embodiments, click chemistry can be used to attach the linker to an active agent (e.g. Diels-Alder reaction, Huigsen 1,3-dipolar cycloaddition, nucleophilic substitution, carbonyl chemistry, epoxidation, dihydroxylation, etc.).

### Example 1: Description of HPLC Analytical Methods

Analysis of the products by C18 Reverse Phase HPLC (Method A) The HPLC analysis was carried out on SunFire™ C18 reverse phase column (4.6×50 mm, 3.5 um) (Waters Corp., Millford, Mass.) with a mobile phase consisting of water +0.01% TFA (solvent A) and acetonitrile+0.01% trifluoroacetic acid (TFA) (solvent B) at a flow rate of 2.0 mL/minute and column temperature of 50° C. The injection volume was 10 μL and the analyte was detected using UV at 220 and 254 nm. The initial gradient was 5% solvent B, increased to 95% solvent B within 1.4 minutes, then maintained at 95% solvent B for 1.6 minutes. All solvents were HPLC grade.

Analysis of the product by C18 Reverse Phase HPLC (RPHPLC) (Method B). RPHPLC analysis was carried out on Zorbax® Eclipse XDB-C18 reverse phase column (4.6×100 mm, 3.5 um, Agilent PN: 961967-902, Agilent Technologies, Santa Clara, Calif.) with a mobile phase consisting of water +0.1% TFA (solvent A) and acetonitrile+0.1% TFA (solvent B) at a flow rate of 1.5 mL/minute and column temperature of 35° C. The injection volume was 10 μL and the analyte was detected using UV at 220 and 254 nm. The initial gradient was 5% solvent B, increased to 95% solvent B within 6 minutes, and maintained at 95% solvent B for 2 minutes, then returned to 5% solvent B and maintained for 2 minutes.

### Example 2: Synthesis of acetate 6-(2,5-dioxo-2H-pyrrol-1(5H)-yl) hexanoate oxaliplatin

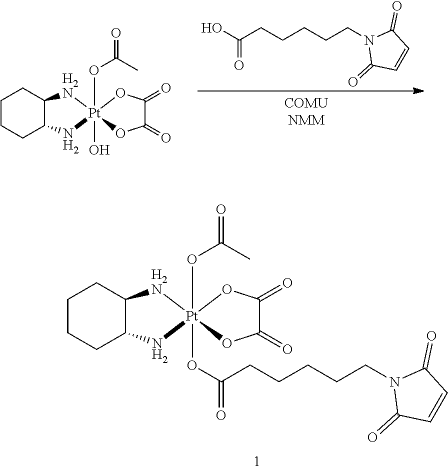

Hydroxy (acetoxy) oxaliplatin (150 mg, 0.317 mmol, 1.0 equiv.), 6-(2,5-Dioxo-2,5-dihydro-1H-pyrrol-1-yl)hexanoic acid (73.6 mg, 0.349 mmol, 1.10 equiv.) and 1-cyano-2-ethoxy-2-oxoethylidenaminooxy)dimethylamino-morpholino-carbenium hexafluorophosphate (COMU®) (149 mg, 0.349 mmol, 1.10 equiv.) were mixed in dimethylformamide (DMF) (6.3 mL) and N-methylmorpholine (38 uL, 0.349 mmol, 1.1 equiv.) was added. The reaction mixture was stirred at room temperature for 2 hours. The reaction was then concentrated to dryness and the residue was loaded directly onto a C18 column (30 g) eluting with 0-30% MeCN/H₂O gradient over 15 minutes. Fractions containing the product were concentrated on a rotavap before the residue was triturated in CH₂Cl₂ and methyl tert-butyl etherTBME, yielding a yellow solid. The product was re-submitted to purification using a silica gel column (4 g), eluted with 0-10% MeOH/CH₂Cl₂ gradient over 15 minutes. Fractions containing the product were combined, concentrated, diluted with MeCN/H₂O and lyophilized to provide the product as a white solid (24.0 mg, 11% yield, 97% pure); ¹H NMR (500 MHz, D20) δ 8.92-8.37 (m, 4H), 7.01 (s, 2H), 3.46-3.41 (m, 2H), 2.92-2.80 (m, 2H), 2.37-2.29 (m, 2H), 2.27-2.21 (m, 2H), 1.94 (s, 3H), 1.67-1.56 (m, 4H), 1.55-1.45 (m, 4H), 1.33-1.26 (m, 2H), 1.26-1.20 (m, 2H); HPLC-MS 97%. m/z for C₂₀H₂₉N₃O₁₀Pt [(M+1)+]=667.3.

### Example 3: Synthesis of a Pt(IV)M monomaleimide, Compound 2

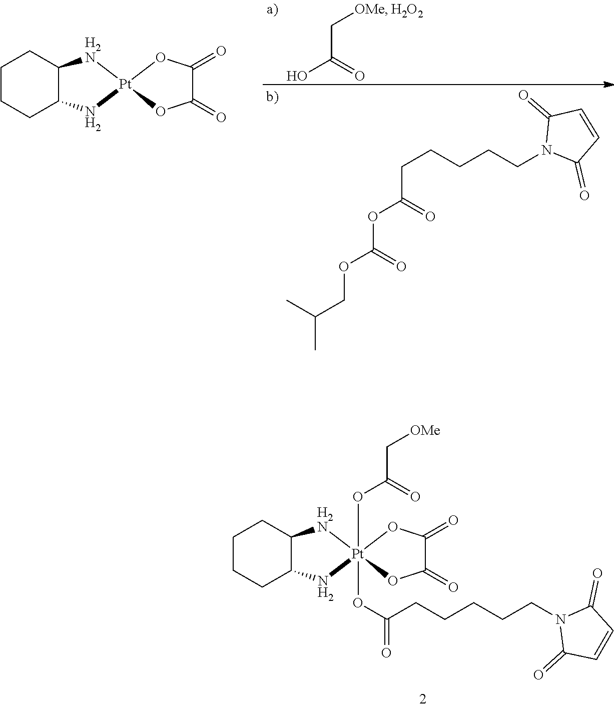

Step 1: Oxaliplatin (300 mg, 0.76 mmol, 1.0 equiv.) was suspended in methoxyacetic acid (3.40 g, 37.8 mmol, 50 equiv.), then hydrogen peroxide (30% w/w in water, 0.13 mL, 3.8 mmol, 5.0 equiv.) was added and solution was stirred for 1 hour. Diethyl ether was added and resulting precipitate was filtered and dried to afford a white solid (400 mg).

Step 2: 6-maleimidohexanoic acid (126 mg, 0.596 mmol, 1.0 equiv.) was suspended in tetrahydrofuran(THF) (5 mL) then 4-methyl morpholine (65 uL, 0.596 mmol, 1.0 equiv.) was added followed by isobutyl chloroformate (77 uL, 0.596 mmol, 1.0 equiv.). The solution was stirred at room temperature for 1 hour, then water (10 mL) and EtOAc (10 mL) were added and the layers were separated. The organic layer was concentrated on a rotavap to dryness. The methoxyacetate hydroxy oxaliplatin (300 mg, 0.596 mmol, 1.00 equiv.) was dissolved in DMF (1 mL) then activated ester was added and solution was stirred at room temperature for 2 hours. The crude product was purified by reverse phase chromatography (MeCN/H₂O, 0.2% AcOH) to afford the desired product, which was lyophilized, resulting in a lyophilized powder (115 mg, 28% yield). HPLC/MS (Method A): 1.333 minutes, M+H=696.2, 697.2, 698.3.

### Example 4: Synthesis of a Pt(IV)M monomaleimide, Compound 3

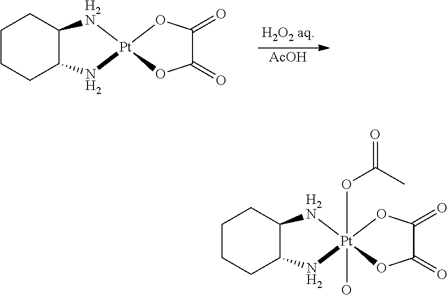

Another example of a compound taught herein was synthesized starting with oxaliplatin (1.0 g, 2.52 mmol, 1 equiv.) that was dissolved in acetic acid (6 mL, 100 mmol, 40 equiv.) and reacted with 30% H₂O₂ (1 mL, 12.6 mmol, 5 equiv.). The reaction mixture was covered with aluminium foil and stirred at room temperature for 24 hours. Following this reaction, more acetic acid (3 mL, 50 mmol, 20 equiv.) and 30% H₂O₂ (1 mL, 12.6 mmol, 5 equiv.) were added and the reaction mixture was stirred for an additional 24 hours. The suspension was filtered and the white solid residue was washed with diethylether (Et₂O) to yield hydroxy(acetoxy)oxaliplatin (878 mg, 74% yield, 70% pure). ¹H NMR (500 MHz, DMF-d7) δ 10.42 (brs, 2H), 8.93 (m, 1H), 8.39 (brs, 1H), 7.96 (brs, 1H), 7.26 (brs, 1H), 2.91-2.80 (m, 2H), 2.31-2.22 (m, 2H), 1.98 (s, 3H), 1.89 (s, 3H), 1.73-1.49 (m, 4H), 1.34-1.21 (m, 2H); HPLC-MS 91%, m/z for C₁₀H₁₈N₂O₇Pt [(M+H)+]=474.2.

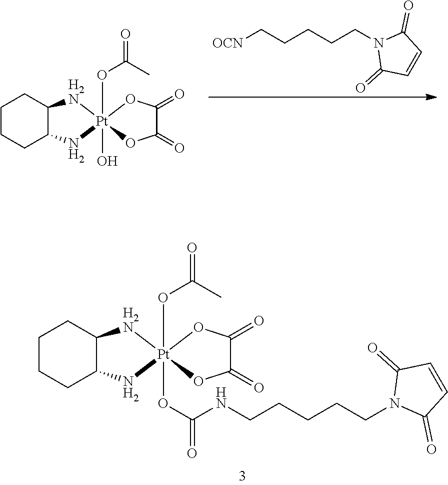

Hydroxy(acetoxy)oxaliplatin (200 mg, 0.422 mmol, 1.0 equiv.) and DMF (1.10 mL) were loaded in a round bottom flask. At room temperature, isocyanate (176 mg, 0.845 mmol, 2.0 equiv.) in DMF (1.0 mL) was added in one portion and the reaction was stirred at room temperature for 2 hours. HPLC trace demonstrated the reaction to be completed producing also ˜30% of bis-addition product due to the presence of bishydroxyoxaliplatin in the starting material. Water (8.0 mL) was added to the reaction mixture and the solution was loaded directly onto a C18 column (60 g). The reaction mixture was purified using a 0-40% MeCN/H₂O gradient over 12 column volumes. The compound eluted at 26% MeCN/H₂O. Pure fractions were combined and lyophilized to provide the product as a white solid (157.6 mg, 55%); ¹H NMR (500 MHz, DMF-d7) δ 10.10 (s, 1H), 8.92-8.63 (m, 2H), 8.35 (s, 1H), 7.07 (t, J=5.6 Hz, 1H), 7.01 (s, 2H), 3.44 (t, J=7.2 Hz, 2H), 3.09-2.95 (m, 2H), 2.90-2.84 (m, 2H), 2.33 (t, J=11.4 Hz, 2H), 1.94 (s, 3H), 1.67-1.14 (m, 12H); HPLC-MS 98.6%. m/z for C₂₀H₃₀N₄O₁₀Pt [(M+1)⁺]=682.3.

### Example 5: Synthesis of a Pt(IV)M monomaleimide, Compound 4

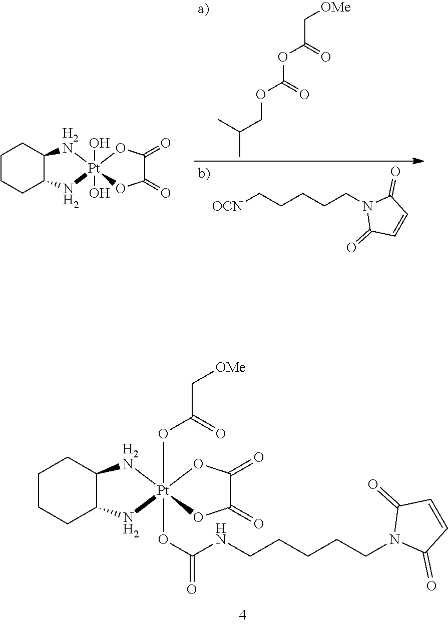

Methoxyacetic acid (42 mg, 0.464 mmol, 1.00 equiv.) was suspended in THF (3 mL) then 4-methyl morpholine (51 uL, 0.464 mmol, 1.00 equiv.) was added followed by isobutyl chloroformate (60 uL, 0.464 mmol, 1.00 equiv.). The solution was stirred at room temperature for 1 hour, then water (5 mL) and EtOAc (5 mL) were added and the layers were separated. The organic layer was concentrated on a rotavap to dryness. Dihydroxyoxaliplatin (200 mg, 0.464 mmol, 1.00 equiv.) was suspended in DMSO (4 mL) then the activated ester was added and solution was stirred at room temperature for 3 days. The remaining solid was filtered, then diethyl ether (10 mL) was added to the filtrate and stirred for 5 minutes before layers were separated. Acetone was added (20 mL) to the DMSO layer, which caused a solid to precipitate. After stirring for 5 minutes, the solid was filtered, washed with acetone, and dried under vacuum to afford an off-white solid (105 mg).

2-Methoxyacetate hydroxyl oxaliplatin (105 mg, 0.21 mmol, 1.0 equiv.) was dissolved in DMF (1 mL) then 1-(5-isocyanatopentyl)-1H-pyrrole-2,5-dione (65 mg, 0.31 mmol, 1.5 equiv.) was added and the solution was stirred at room temperature for 16 hours. The crude product was purified by reverse phase chromatography (MeCN/H₂O, 0.2% AcOH) to afford desired product as a lyophilized powder (67 mg). HPLC/MS (Method A): 1.203 minutes, M+H=711.2, 712.2, 713.3.

### Example 6: Synthesis of a Pt(IV)M monomaleimide, Compound 5

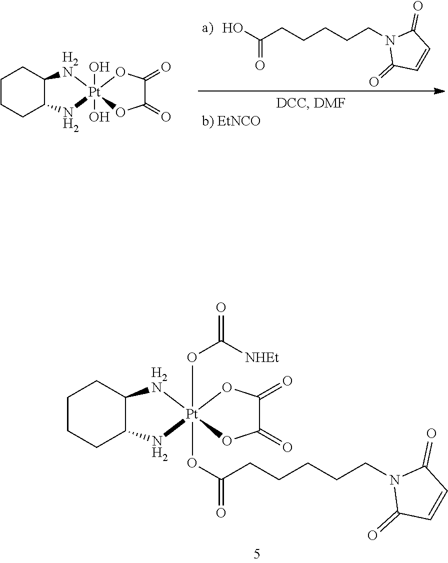

6-maleimidohexanoic acid (844 mg, 4.0 mmol, 2.0 equiv.) and dicyclohexylcarbodiimide (DCC) (822 mg, 4.0 mmol, 2.0 equiv.) were charged in a vial and dissolved in DMF (10 mL). The solution was stirred for 30 minutes, by which time a precipitate had formed. The solid was filtered, the filtrate was added to dihydroxyoxaliplatin (862 mg, 2.0 mmol, 1.0 equiv.) suspended in DMF (10 mL) and solution was stirred at room temperature for 4 hours. The suspension was filtered, then ethyl isocyanate (EtNCO) (206 mL, 4.0 mmol, 2.0 equiv.) was added to filtrate and solution was stirred at room temperature for 30 minutes. Solvent was then evaporated and the residue was purified on silica gel (2-7% MeOH/DCM (dichloromethane)). Concentrated pure fractions were dissolved in MeOH (2 mL) and added to TBME (50 mL) to afford a white precipitate that was filtered and dried. The desired product was obtained as a white solid (340 mg). HPLC/MS (Method B): 3.81 minutes, M+H=695, 696, 697.

### Example 7: Synthesis of a Pt(IV)M monomaleimide, Compound 6

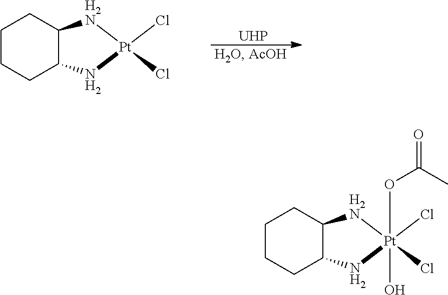

Hydroxy(acetoxy)(DACH)PtCl₂: (DACH)PtCl₂ (1.00 g, 2.63 mmol, 1.0 equiv.) and urea hydroperoxide (UHP) (1.24 g, 13.15 mmol, 5.0 equiv.) were loaded in a round bottom flask. AcOH (13 mL) and H₂O (13 mL) were added and the reaction was stirred at room temperature overnight. The yellow solution reaction mixture was then concentrated to dryness. We noted that starting material was still present according to HPLC-MS traces. The crude solid obtained was suspended in about 10 mL of MeOH and DCM was slowly added to obtain a suspension. After stirring for 30 minutes, filtration of the solid over a Buchner funnel provided the desired compound as an off-white solid (410 mg, 34.2%). HPLC-MS 93%, m/z for C₈H₁₈C₁₂N₂O₃Pt [(M+1)⁺]=457.2.

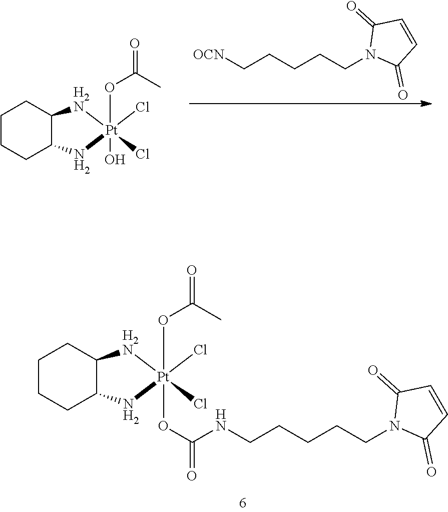

Hydroxy(acetoxy)(DACH)PtCl₂ (200 mg, 0.438 mmol, 1.0 equiv.) and DMF (3.0 mL) were loaded in a round bottom flask. At room temperature, 1-(5-isocyanatopentyl)-1H-pyrrole-2,5-dione (273 mg, 1.31 mmol, 3.0 equiv.) in DMF (1.40 mL) was added in one portion and the reaction stirred at room temperature overnight. The next day, HPLC traces demonstrated the reaction to be completed. The reaction mixture was loaded directly onto a C18 column (60 g) and purified using 0-60% MeCN/H₂O gradient over 15 column volumes. The compound eluted at 32% MeCN/H₂O. Pure fractions were combined and lyophilized to provide the product as a white solid (182 mg, 63%). ¹H NMR (500 MHz, DMF-d7) δ 10.94 (s, 1H), 9.74 (s, 1H), 8.43 (s, 1H), 8.11 (s, 1H), 7.05 (t, J=6.0 Hz, 1H), 7.03-7.00 (m, 2H), 3.47-3.42 (m, 2H), 3.10-2.94 (m, 2H), 2.90-2.80 (m, 1H), 2.44-2.30 (m, 2H), 1.93 (s, 3H), 1.69-1.37 (m, 9H), 1.37-1.15 (m, 4H); HPLC-MS 95.4%, m/z for C₁₈H₃₀Cl₂N₄O₆Pt [(M+1)⁺]=665.2.

### Example 8: Synthesis of a Pt(IV)M monomaleimide, Compound 7

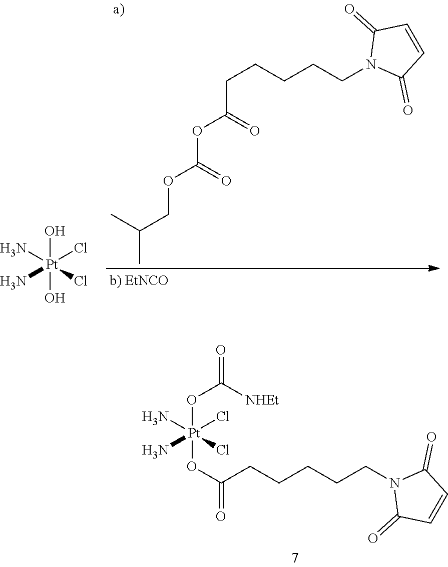

6-Maleimidohexanoic acid (285 mg, 1.35 mmol, 1.00 equiv.) was suspended in THF (10 mL) then 4-methyl morpholine (148 uL, 1.35 mmol, 1.00 equiv.) was added followed by isobutyl chloroformate (175 uL, 1.35 mmol, 1.00 equiv.). The solution was stirred at room temperature for 1 hour, then water (20 mL) and EtOAc (20 mL) were added and layers were separated. Dihydroxy cisplatin (450 mg, 1.35 mmol, 1.00 equiv.) was suspended in DMSO (8 mL) then activated ester was added and the solution was stirred at room temperature for 3 days. Ethyl isocyanate (500 uL, 9.7 mmol, 7.0 equiv.) was added as a solution in DMF (2 mL) and solution was stirred at room temperature for 1 hour. The product was purified by reverse phase chromatography (MeCN/H₂O, 0.2% AcOH) to afford the desired product as a lyophilized powder (160 mg). HPLC/MS (method A): 1.473 minutes, M+H=597.1, 598.1 599.1.

### Example 9: Synthesis of a Pt(IV)M monomaleimide, Compound 8

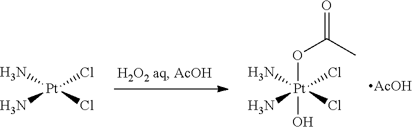

Hydroxy(acetoxy)cisplatin was synthesized by suspending cisplatin (5.0 g, 16.7 mmol, 1.0 equiv.) in acetic acid (40 mL, 667 mmol, 40 equiv.) and treating with 30% H₂O₂ (6.5 mL, 83.5 mmol, 5 equiv.). The reaction mixture was covered with aluminium foil and stirred at room temperature for 24 hours yielding a yellow solid that was filtered and washed with Et₂O to afford 2.86 g of hydroxy(acetoxy)cisplatin acetic acid complex (37% yield). ¹H NMR (500 MHz, DMSO) δ 6.57-6.10 (m, 6H), 2.33 (s, 3H), 2.29 (s, 3H); HPLC-MS 100%, m/z for C₂H₁₀Cl₂N₂O₃Pt [(M+H)⁺]=377.0.

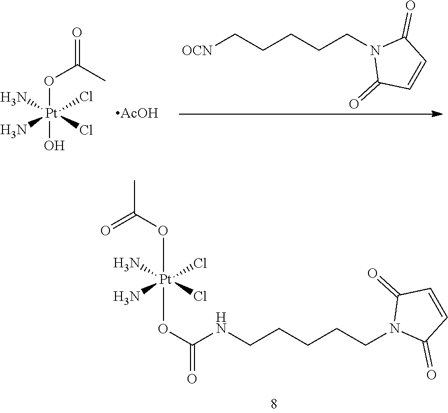

Hydroxy(acetoxy)cisplatin acetic acid complex (100 mg, 0.229 mmol, 1.00 equiv) and DMF (1.0 mL) were loaded in a round bottom flask. At room temperature, 1-(5-isocyanatopentyl)-1H-pyrrole-2,5-dione (111 mg, 0.532 mmol, 2.32 equiv.) in DMF (0.8 mL) was then added in one portion and reaction stirred at room temperature overnight. Water (4.0 mL) was added to the reaction mixture and the solution was loaded directly onto a C18 column (60 g). The reaction mixture purified using a 0-80% MeCN/H₂O gradient over 15 column volumes. The compound eluted at 30% MeCN/H₂O. Pure fractions were combined and lyophilized to provide compound 8 as a white solid (117.0 mg, 87% yield). ¹H NMR (500 MHz, DMF-d7) δ 7.19-6.75 (m, 9H), 3.45 (t, J=7.2 Hz, 2H), 3.04-2.95 (m, 2H), 1.90 (s, 3H), 1.59-1.50 (m, 2H), 1.49-1.37 (m, 2H), 1.34-1.20 (m, 2H); HPLC-MS 96.7%. m/z for C₁₂H₂₂Cl₂N₄O₆Pt [(M+H)⁺]=585.2.

### Example 10: Synthesis of a Pt(IV)M monomaleimide, Compound 9

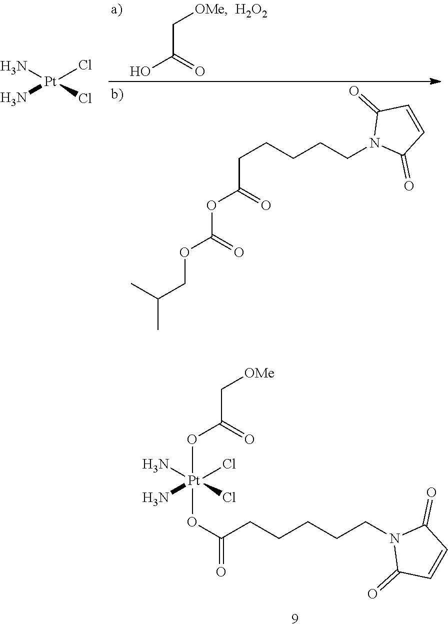

Step 1: Cisplatin (300 mg, 1.00 mmol, 1.00 equiv.) was suspended in methoxyacetic acid (4.5 g, 50 mmol, 50 equiv.), then hydrogen peroxide (30% w/w in water, 0.57 mL, 5 mmol, 5.0 equiv.) was added and the solution was stirred for 2 days. Diethyl ether was then added and the resulting precipitate was filtered and dried to afford a mixture of dimethoxyacetate cisplatin and methoxyacetate hydroxy cisplatin (1.5:1). The white solid (400 mg) was suspended in DMF (10 mL) and used for subsequent steps. To prepare an activated ester, 6-maleimidohexanoic acid (316 mg, 1.5 mmol, 1.0 equiv.) was suspended in THF (10 mL) then 4-methyl morpholine (165 uL, 1.5 mmol, 1.0 equiv.) was added followed by isobutyl chloroformate (194 uL, 1.5 mmol, 1.0 equiv.). The solution was stirred at room temperature for 1 hour, then water (20 mL) and EtOAc (20 mL) were added and the layers were separated. The organic layer was concentrated on a rotavap to dryness. The resulting activated ester was dissolved in DMF (2 mL), added to methoxyacetate hydroxy cisplatin solution and stirred at room temperature for 2 hours then purified by reverse phase chromatography (MeCN/H₂O, 0.2% AcOH) to afford the desired product as a lyophilized powder (115 mg). HPLC/MS (method A): 1.199 minutes, M+H=599.0, 600.0, 601.1.

### Example 11: Synthesis of a Pt(IV)M monomaleimide, Compound 10

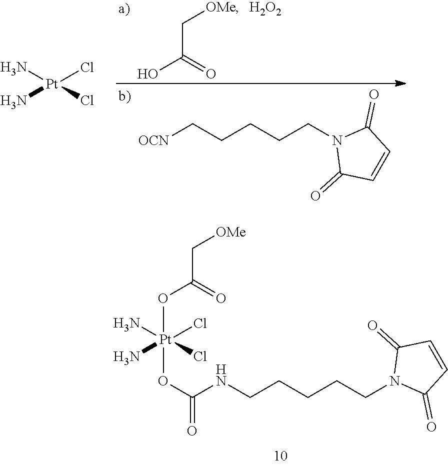

Step 1: Cisplatin (250 mg, 0.83 mmol, 1.00 equiv.) was suspended in methoxyacetic acid (3.75 g, 41.7 mmol, 50.0 equiv.), then hydrogen peroxide (30% w/w in water, 0.47 mL, 4.17 mmol, 5.0 equiv.) was added and solution was stirred for 2 days. Diethyl ether was added and resulting precipitate was filtered and dried to afford a mixture of dimethoxyacetate cisplatin and methoxyacetate hydroxycisplatin (1.5:1). The white solid (350 mg) was dissolved in DMF (1 mL) then isocyanate (260 mg, 1.25 mmol, 1.50 equiv.) was added and the solution was stirred at room temperature for 2 hours. Crude product was purified by reverse phase chromatography (MeCN/H₂O, 0.2% AcOH) to afford desired product as a lyophilized powder (139 mg). HPLC/MS (Method A): 1.184 minutes, M+H=614.1, 615.1, 616.1.

### Example 12: Synthesis of a Pt(IV)M monomaleimide, Compound 11

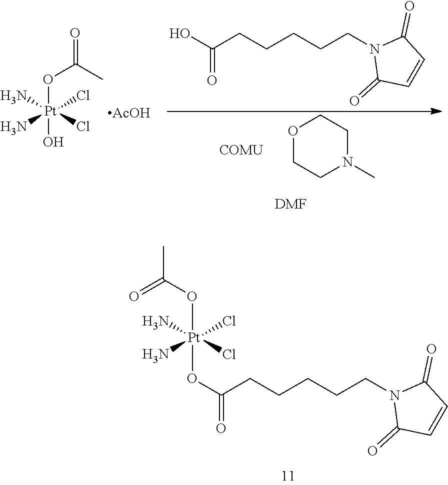

To hydroxy(acetoxy)cisplatin acetic acid complex (200 mg, 0.459 mmol, 1.00 equiv.) suspended in DMF (0.035 M, 15 mL) was added 6-maleimidohexanoic acid (130 mg, 0.61 mmol, 1.33 equiv.) followed by N-methylmorpholine (67 μL, 0.61 mmol, 1.33 equiv.) and COMU peptide coupling reagent (263 mg, 0.61 mmol, 1.33 equiv.). The reaction mixture was stirred at room temperature for 15 hours. The resulting clear yellow solution was concentrated under reduced pressure. The resulting residue was diluted in water (20 mL) and washed twice with methyl tertiary-butyl ether (MTBE) (2×15 mL). The aqueous layer was concentrated under reduced pressure to an approximate residual volume of 5 mL. This solution containing the product was injected onto a pre-packed C-18 cartridge (30 g) and eluted on a reverse phase system with a 15-50% (MeCN/water) 15 minute gradient. Pure collected fractions were combined and concentrated under reduce pressure and residual water was removed by lyophilization to afford compound 11 as an off-white solid (119 mg, 46%). ¹H NMR (500 MHz, DMF-d7) δ (ppm): 7.02 (s, 2H), 7.03-6.67 (m, 6H), 3.44 (t, J=7.3 Hz, 2H), 2.22 (t, J=7.3 Hz, 2H), 1.90 (s, 3H), 1.57-1.46 (m, 4H), 1.31-1.23 (m, 2H). HPLC-MS 99%, m/z for C₁₂H₂₁Cl₂N₃O₆Pt [(M+H)+]=570.2.

### Example 13: Synthesis of a Pt(IV)M monomaleimide, Compound 12

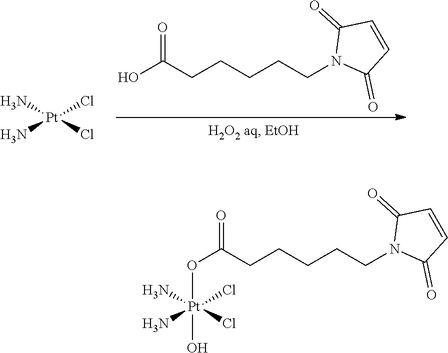

Synthesis of hydroxy(6-(2,5-dioxo-2H-pyrrol-1(5H)-yl)hexanoate)cisplatin. Cisplatin (200 mg, 0.67 mmol, 1.00 equiv.) was suspended in ethanol (1 mL) and was added the 6-(2,5-dioxo-2,5-dihydro-1H-pyrrol-1-yl)hexanoic acid (703 mg, 3.33 mmol, 5.00 equiv.) followed by 30% H₂O₂ (160 uL, 2.01 mmol, 3.00 equiv.). The reaction mixture was covered with aluminum foil and stirred at room temperature for 60 hours. The reaction mixture was concentrated on a rotavap to dryness, dissolved in DMF, loaded directly onto a C18 column (30 g) and eluted with a 0-40% MeCN/H₂O gradient over 15 minutes. Pure fractions were combined and lyophilized to provide hydroxy(6-(2,5-dioxo-2H-pyrrol-1(5H)-yl)hexanoate)cisplatin as a yellow solid (90.4 mg); HPLC-MS m/z for C₁₀H₁₉Cl₂N₃O₅Pt [(M+H)+]=528.2.

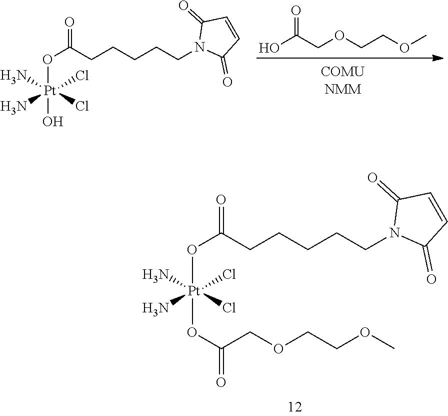

### Synthesis of 2-(2-Methoxyethoxy)acetate 6-(2,5-dioxo-2H-pyrrol-1(5H)-yl)hexanoatecisplatin

Hydroxy(6-(2,5-dioxo-2H-pyrrol-1(5H)-yl)hexanoate)cisplatin (140 mg, 0.265 mmol, 1.00 equiv.), 3,6-dioxaheptanoic acid (39.0 mg, 0.292 mmol, 1.10 equiv.) and COMU (125 mg, 0.292 mmol, 1.10 equiv.) were mixed in DMF (5 mL) followed by the addition of N-methylmorpholine (32 uL, 0.29 mmol, 1.1 equiv.). The reaction mixture was stirred at room temperature overnight. The reaction mixture was then diluted with water and the aqueous layer was washed with MTBE. The aqueous layer was concentrated to dryness, dissolved in water, loaded directly onto a C18 column (30 g) and eluted using a 0-30% MeCN/H₂O gradient over 15 minutes. Fractions containing the product were concentrated on a rotavap to dryness. The residue was adsorbed on silica gel and purified using a silica gel column (24 g), eluted using 97-70% MeCN/H₂O gradient over 15 minutes. Fractions containing the product were concentrated, the residue was adsorbed on silica gel and purified using a silica gel column (4 g), eluted using 0-10% MeOH/CH₂Cl₂ gradient over 15 minutes. Fractions containing the product were concentrated and the residue was diluted with MeCN/H₂O and lyophilized to provide the product, compound 12, as a yellow solid (31.8 mg, 19% yield); ¹H NMR (500 MHz, D20) δ 6.85 (s, 2H), 4.26 (s, 2H), 3.75-3.72 (m, 2H), 3.67-3.64 (m, 2H), 3.55-3.50 (m, 2H), 3.40 (s, 3H), 2.45-2.38 (m, 2H), 1.62-1.55 (m, 4H), 1.35-1.25 (m, 2H); HPLC-MS 97%. m/z for C₁₅H₂₇Cl₂N₃O₈Pt [(M+H)+]=644.2.

### Example 14: Synthesis of a Pt(IV)M monomaleimide, Compound 13

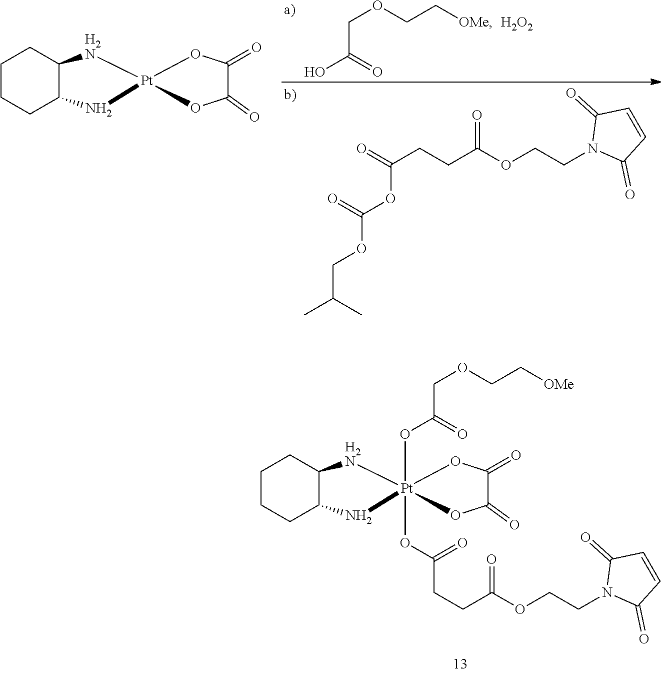

Oxaliplatin (2.05 g, 5.00 mmol, 1.00 equiv.) was suspended in 2-methoxyethoxyacetic acid (22.7 mL, 200 mmol, 40.0 equiv.), then hydrogen peroxide (30% w/w in water, 0.775 mL, 25 mmol, 5.0 equiv.) was added and solution was stirred for 16 hours. MTBE (175 mL) was added and the filtrate was decanted. The gummy residue was dissolved in DMF (5 mL) and added dropwise onto EtOAc (125 mL). The resulting precipitate was filtered, rinsed with EtOAc (3×20 mL) and dried under vacuum to afford a white solid (1.75 g). HPLC/MS (method B): 2.29 minutes, M+H=547, 548, 549 and 2.81 minutes, M+H=663, 664, 665. The solid consists of a 1:1 mixture of desired product and bis acylation product and was used in that composition for next step.

2-Hydroxyethyl maleimide (120 mg, 0.85 mmol, 2.00 equiv.) was suspended in THF (4.0 mL) then N-methyl morpholine (93 uL, 0.85 mmol, 2.0 equiv.) was added followed by succinic anhydride (85 mg, 0.85 mmol, 2.0 equiv.). The solution was stirred at room temperature for 16 hours, then N-methyl morpholine (93 uL, 0.85 mmol, 2.0 equiv.) was added followed by isobutyl chloroformate (110 uL, 0.85 mmol, 2.0 equiv.) and the solution was stirred at room temperature for 45 minutes. Water (10 mL) and ethyl acetate (10 mL) were added and the layers were separated. Solvent was then evaporated, then the activated ester was dissolved in DMF and hydroxy(2-methylethoxyacetoxy)oxalplatin(IV) (233 mg, 0.43 mmol, 1.0 equiv.) was added and solution was stirred sat 50° C. for 18 hours. Solvent was evaporated from this mixture and the crude product was purified on silica gel (0-8% MeOH/DCM gradient) to afford the desired product with residual MeOH. The residue was dissolved in MeOH (2 mL) and the resulting solution was added to TBME (50 mL) to obtain a white precipitate (compound 13) that was filtered and dried under vacuum. Compound 13 was obtained as an off-white solid (131 mg, 40% yield). HPLC/MS (method B): 3.41 minutes, M+H=771.

### Example 15: Synthesis of a Pt(IV)M monomaleimide, Compound 14

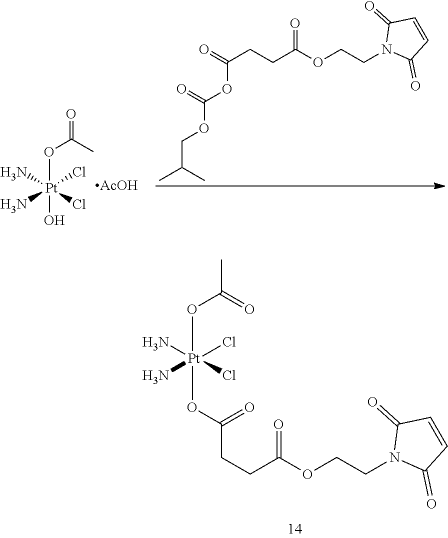

2-Hydroxyethyl maleimide (120 mg, 0.85 mmol, 2.3 equiv.) was suspended in THF (4.0 mL) then N-methyl morpholine (93 uL, 0.85 mmol, 2.3 equiv.) was added followed to succinic anhydride (85 mg, 0.85 mmol, 2.3 equiv.). Solution was stirred at room temperature for 16 hours, then N-methyl morpholine (93 uL, 0.85 mmol, 2.3 equiv.) was added followed by isobutyl chloroformate (110 uL, 0.85 mmol, 2.3 equiv.) and solution was stirred at room temperature for 45 minutes. Water (10 mL) and ethyl acetate (10 mL) were added and layers were separated. Solvent was evaporated, then activated ester was dissolved in DMF and hydroxy(acetoxy)cisplatin(IV) acetic acid complex (160 mg, 0.367 mmol, 1.00 equiv.) was added and solution was stirred at 50° C. for 18 hours. The solvent was evaporated and crude was purified on silica gel (3-8% MeOH/DCM gradient) to afford the desired product with residual MeOH. The residue was dissolved in MeOH (1 mL) and solution was added to TBME (50 mL) to obtain a white precipitate that was filtered and dried under vacuum. Compound 14 was obtained as an off-white solid (83 mg, 38% yield). HPLC/MS (method B): 3.21 minutes, M+H=600.

### Example 16: Synthesis of a Pt(IV)M monomaleimide, Compound 15

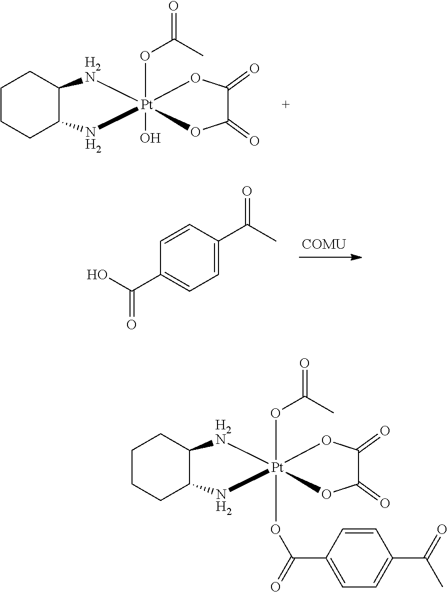

Acetoxyoxaliplatin(4-acetylphenyl)carboxylate: Acetoxy(hydroxyl)oxaliplatin (409 mg, 0.86 mmol, 1.00 equiv.), 4-acetylbenzoic acid (170 mg, 1.03 mmol, 1.20 equiv.) and COMU (444 mg, 1.03 mmol, 1.20 equiv.) were suspended in DMF (0.1 M, 8 mL) and N-methylmorpholine (114 uL, 1.03 mmol, 1.20 equiv.) was added at room temperature. The reaction mixture was stirred at room temperature for 16 hours. The reaction mixture was concentrated and the residue was suspended on silica gel and purified by normal phase chromatography using a silica gel column (40 g) eluted with 5-20% MeOH/CH₂Cl₂ gradient over 15 minutes. Pure fractions were combined and concentrated in vacuum to provide the product as an off-white solid (328 mg, 61%, 94.8% pure). HPLC-MS 94.8%, m/z for C₁₉H₂₄N₂O₉Pt [(M+H)+]=620.2.

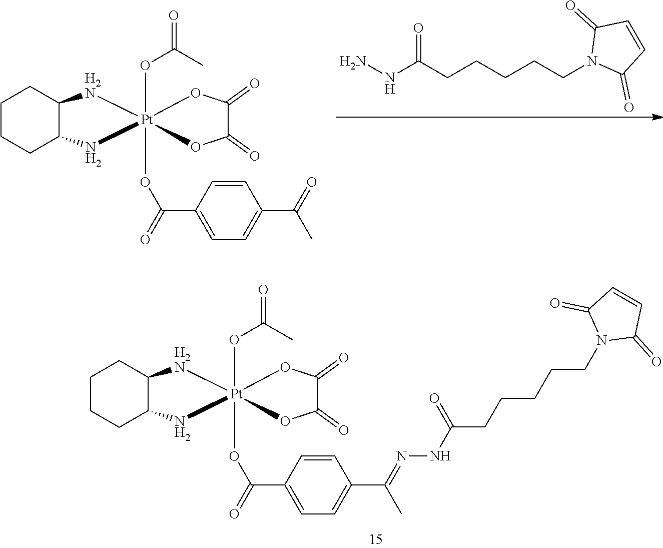

Acetate 4-(1-(2-(6-(2,5-dioxo-2H-pyrrol-1(5H)-yl)hexanoyl)hydrazono)ethyl)benzoate oxaliplatin was synthesized using acetoxyoxalplatin(4-acetylphenyl)carboxylate (328 mg, 0.529 mmol, 1.00 equiv.) that was dissolved in DMF (0.05 M, 10 mL) and treated with 6-(2,5-dioxo-2,5-dihydro-1H-pyrrol-1-yl)hexanehydrazide TFA salt (359 mg, 1.05 mmol, 2.00 equiv.). The reaction mixture was stirred at room temperature for 16 hours. MTBE was added to the reaction mixture until a gum was formed and the solvent was decanted. To the gummy residue was added more MTBE and the mixture was incubated in an ultrasonic bath until the gum turned into a yellow solid. The solid was filtered and rinsed with MTBE to afford compound 15(126 mg, 29%, 93.2% pure); 1H NMR (500 MHz, DMF-d7) δ 10.50 (s, 0.4H), 10.42 (s, 0.6H), 8.95-8.44 (m, 4H), 7.92-7.88 (m, 4H), 7.03 (s, 0.8H), 7.02 (s, 1.2H), 3.62-3.35 (m, 6H), 2.46-2.33 (m, 5H), 1.98 (s, 3H), 1.78-1.50 (m, 8H), 1.43-1.22 (m, 4H); HPLC-MS 93.2%, m/z for C29H37N5O11Pt [(M+H)+]=828.3

### Example 17: Synthesis of a Pt(IV)M monomaleimide, Compound 16

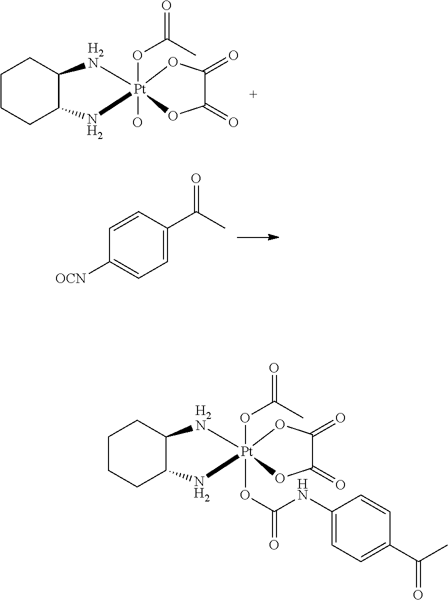

Acetoxyoxaliplatin(4-acetylphenyl)carbamate was first synthesized using acetoxy(hydroxyl)oxaliplatin (243 mg, 0.51 mmol, 1.00 equiv.) and 4-acetylphenylisocyanate (124 mg, 0.77 mmol, 1.50 equiv.) dissolved in DIVIF (0.1 M, 5 mL). The reaction mixture was stirred at room temperature overnight. The reaction mixture was concentrated and impregnated on silica gel. The crude product was purified by normal phase chromatography using silica gel column (40 g) eluted with 5-20% MeOH/CH₂Cl₂ gradient over 15 minutes. Pure fractions were combined and concentrated under vacuum to provide the product as a yellow solid (241 mg, 74%, 83% pure). HPLC-MS 83.1%, m/z for C₁₉H₂₅N₃O₉Pt [(M+H)+]=635.2.

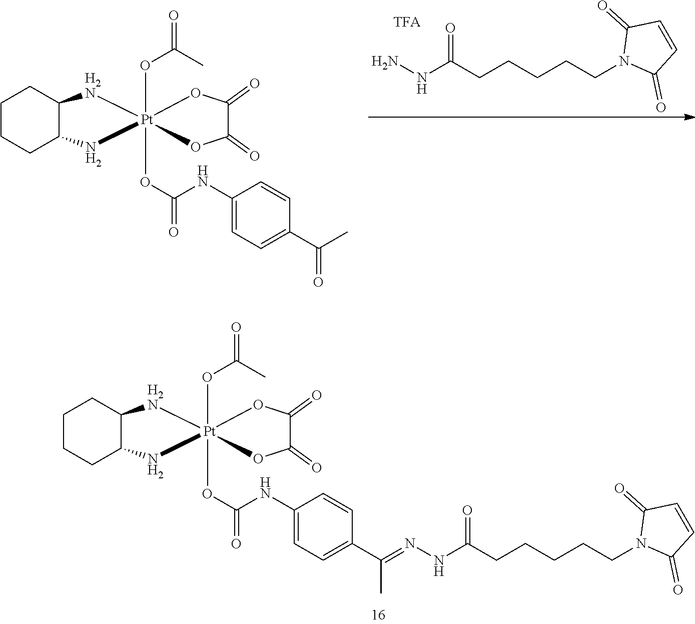

### Synthesis of acetate 4-(1-(2-(6-(2,5-dioxo-2H-pyrrol-1(5H)-1)hexanoyl)hydrazono)ethyl) phenyl carbamate oxaliplatin

Acetoxyoxalplatin(4-acetylphenyl)carbamate (228 mg, 0.36 mmol, 1.00 equiv.) was dissolved in IMF (0.05 M, 7 mL) and treated with 6-(2,5-dioxo-2,5-dihydro-1H-pyrrol-1-yl)hexanehydrazide TFA salt (158 mg, 0.47 mmol, 1.30 equiv.). The reaction mixture was stirred at room temperature for 16 hours. The reaction mixture was concentrated and the residue was triturated with acetonitrile to precipitate the product as a yellow powder. This powder was triturated first with isopropyl alcohol (iPrOH) and then with DCM to afford the desired product (70 mg, 23%, 93.5% pure). HPLC-MS 93.5%, m/z for C₂₉H₃₈N₆O₁₁Pt [(M+H)⁺]=842.3. ¹H NMR (500 MHz, DMF-d7) δ 10.34-10.15 (m, 1H), 9.91-9.66 (m, 1H), 9.37-9.24 (m, 1H), 8.88-8.66 (m, 2H), 8.59-8.48 (m, 1H), 7.80-7.73 (m, 2H), 7.61-7.53 (m, 2H), 7.04-6.98 (m, 2H), 3.50-3.43 (m, 2H), 3.14-3.02 (m, 1H), 2.75-2.70 (m, 1H), 2.43-2.25 (m, 6H), 2.01-1.92 (m, 3H), 1.74-1.53 (m, 9H), 1.42-1.24 (m, 4H).

### Example 18: Synthesis of a Pt(IV)M monomaleimide, Compound 17

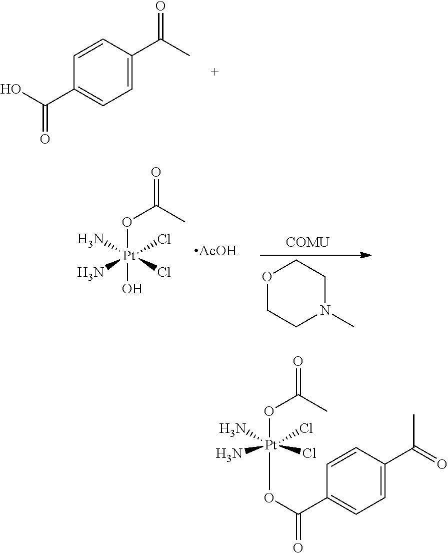

Acetoxycisplatin(4-acetylphenyl)carboxylate: Hydroxy(acetoxy)cisplatin acetic acid complex (500 mg, 1.15 mmol, 1.0 equiv.), 4-acetylbenzoic acid (240 mg, 1.46 mmol, 1.27 equiv.) and COMU (625 mg, 1.46 mmol, 1.27 equiv.) were suspended in DMF (0.05M, 25 mL) followed by N-methylmorpholine (160 uL, 1.46 mmol, 1.27 equiv.) at room temperature. The reaction mixture was stirred at room temperature overnight and the suspension slowly became a yellow solution. The HPLC-MS showed incomplete conversions, and more COMU (205 mg, 0.478 mmol, 0.42 equiv.) and N-methylmorpholine (160 uL, 1.46 mmol, 1.27 equiv.) were added. The reaction mixture was then stirred for an additional 3 hours. The reaction mixture was concentrated and the residue was dissolved in water and purified by reversed phase chromatography using a pre-packed C18 60 g-column, eluted with 0-30% MeCN/H₂O gradient over 15 minutes. Pure fractions were combined and lyophilized to provide the product as a yellow solid (236 mg, 34%, 80% pure). The solid was triturated in MTBE to afford acetoxycisplatin(4-acetylphenyl)carboxylate (180 mg, 30% yield). HPLC-MS 100%, m/z for C₁₁H₁₆Cl₂N₂O₅Pt [(M+H)⁺]=523.1.

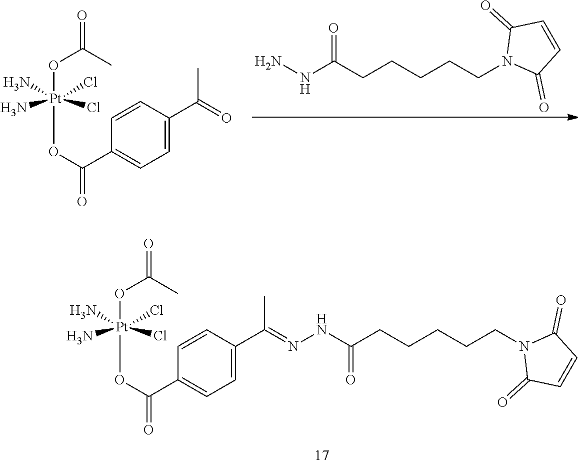

Acetoxycisplatin(4-acetylphenyl)carboxylate (178 mg, 0.341 mmol, 1 equiv.) was dissolved in DMF (0.05 M, 6.8 mL) and treated with 6-(2,5-dioxo-2,5-dihydro-1H-pyrrol-1-yl)hexanehydrazide TFA salt (139 mg, 0.409 mmol, 1.2 equiv.). The reaction mixture was stirred at room temperature for 5 hours. MTBE was added to the reaction mixture until a suspension was obtained and a yellow solid was filtered to afford compound 17 (159 mg, 64%, 97% pure). ¹H NMR (500 MHz, DMF-d7) δ 10.48 (s, 0.3H), 10.40 (s, 0.6H), 7.97-7.92 (m, 2H), 7.91-7.86 (m, 2H), 7.24-6.77 (m, 6H), 7.02 (s, 2H), 3.50-3.44 (m, 2H), 2.77-2.72 (m, 1.4H), 2.44-2.38 (m, 0.6H), 2.40 (s, 2H), 2.37 (s, 1H), 1.94 (s, 3H), 1.73-1.64 (m, 2H), 1.63-1.54 (m, 2H), 1.42-1.29 (m, 2H); HPLC-MS 98%, m/z for C₂₁H₂₉Cl₂N₅O₇Pt [(M+H)⁺]=730.2.

### Example 19: Synthesis of a Pt(IV)M monomaleimide, Compound 18

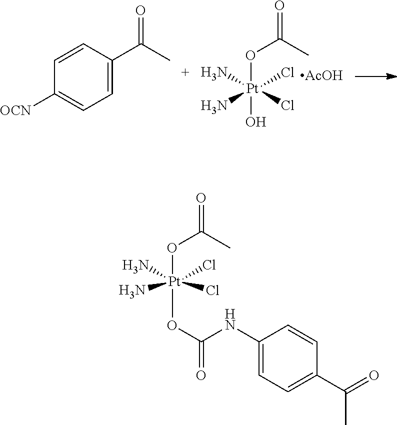

### Synthesis of acetoxycisplatin(4-acetylphenyl)carbamate

Hydroxy(acetoxy)cisplatin acetic acid complex (300 mg, 0.688 mmol, 1.00 equiv.) was suspended in DMF (0.05 M, 16 mL) and before 4-isocyanatoacetophenone (257 mg, 1.60 mmol, 2.33 equiv.) was added at room temperature. The reaction mixture was stirred at room temperature overnight. The next morning the reaction mixture was concentrated to dryness and the residue was triturated in a water and methanol mixture. The suspension was filtered and 340 mg of a yellow solid was obtained containing product and 18% of a 4-acetylphenyl by-product. The solid was dissolved in DMF and purified by reverse phase chromatography using a pre-packed C18 60 g column, eluted with 0-30% MeCN/H₂O gradient over 15 minutes. Pure fractions were combined and lyophilized to provide the product as a yellow solid (218 mg, 59% yield). HPLC-MS 100%, m/z for C₁₁H₁₇Cl₂N₃O₅Pt [(M+H)+]=538.1.

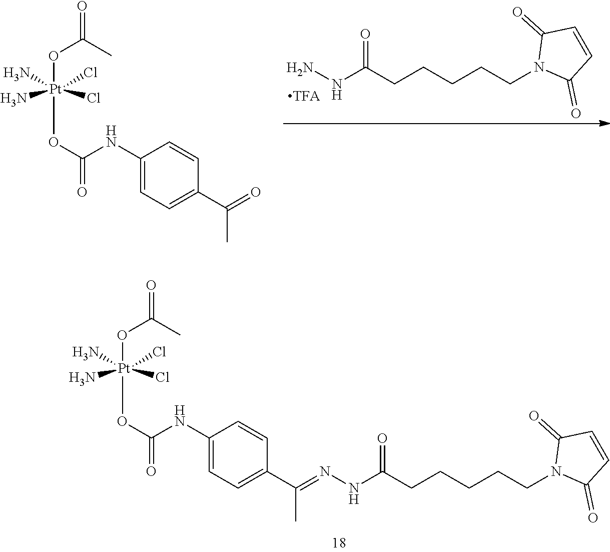

Acetoxycisplatin(4-acetylphenyl)carbamate (215 mg, 0.400 mmol, 1.0 equiv.) was dissolved in DMF (0.05M, 8 mL) and treated with 6-(2,5-dioxo-2,5-dihydro-1H-pyrrol-1-yl)hexanehydrazide TFA salt (163 mg, 0.480 mmol, 1.2 equiv.) at room temperature. The reaction mixture was stirred at room temperature overnight. Dichloromethane was added to the reaction mixture and the suspension was filtered to provide a yellow solid (230 mg, 77% yield, 90.9% pure). The residue obtained was triturated with MeCN to provide compound 18 as a yellow solid (144 mg, 48% yield, 97.3% pure). ¹H NMR (500 MHz, DMF-d7) δ 10.28 (s, 0.4H), 10.15 (s, 0.6H), 9.74 (brs, 1H), 7.79-7.72 (m, 2H), 7.61-7.54 (m, 2H), 7.22-6.81 (m, 6H), 7.02 (s, 2H), 3.50-3.44 (m, 2H), 2.74-2.70 (m, 1.2H), 2.40-2.35 (m, 0.8H), 2.33 (s, 2H), 2.29 (s, 1H), 1.93 (s, 3H), 1.72-1.63 (m, 2H), 1.62-1.53 (m, 2H), 1.42-1.29 (m, 2H); HPLC-MS 97%, m/z for C₂₁H₃₀Cl₂N₆O₇Pt [(M+H)+]=745.2.

### Example 20: In Vitro Inhibition of Cell Proliferation by Pt(IV)M Monomaleimide Compounds

Human cancer cell lines were plated in 96 well plates (Costar) and 24 hours later were treated with a compound for 48-72 hours. Specifically, H460 cells (ATCC) were plated at a concentration of 1,500 cells per well and compound treatment was carried out for 48 hours. Compound starting dose was 20 uM and three-fold serial dilutions were done for a total of ten points. Inhibition of cell proliferation was measured using CellTiter-Glo® reagent using the standard protocol (Promega) and a Glomax® multi+detection system (Promega). Percent proliferation inhibition was calculated using the following formula: % inhibition=(control-treatment)/control*100. Control is defined as vehicle alone. IC50 curves were generated using the nonlinear regression analysis (four parameter) with GraphPad Prism 6.

Selected compounds of the present teachings each has an IC₅₀ between 0.0001 uM and 50 uM. For example, as shown in Table 1, some examples of compounds are less active than cisplatin and have IC₅₀ value greater than 10 uM.

TABLE 1

| Compound | H460 IC₅₀ uM) |
|---|---|
| Cisplatin | 2.0 |
| 4 | 18.9 |
| 5 | >500 |
| 7 | 40.6 |
| 9 | 14.2 |

These data demonstrate that some Pt(IV)M compounds described herein are weakly efficacious compared to cisplatin for inducing cell death in a cancer cell under the conditions of these experiments.

### Example 21: Effect of Pt(IV)M Monomaleimide Compounds on Tumor Growth

Despite the weak efficacy in vitro on cell proliferation compared to cisplatin, applicants assessed the activity of Pt(IV)M monomaleimide compounds in vivo. In these experiments, the ability of compounds to affect the growth of human Calu-6 NSCLC (non-small cell lung cancer) and A2780 ovarian xenografts was tested. All mice were treated in accordance with the OLAW Public Health Service Policy on Human Care and Use of Laboratory Animals and the ILAR Guide for the Care and Use of Laboratory Animals, and were conducted at Charles River Laboratories (Morrisville, N.C.). All in vivo studies were conducted following the protocols approved by the Charles River Institutional Animal Care and Use Committee. For the A2780 in vivo studies, 10 week old female NCR nude mice were inoculated subcutaneously into the right flank with 10 million cells in 1:1 RPMI 1640 (Invitrogen, Carlsbad, Calif.)/Matrigel® (BD Biosciences, San Jose, Calif.). For the Calu-6 in vivo studies, 10 week old female NCR nude mice were inoculated subcutaneously into the right flank with 5 million cells in 1:1 RPMI 1640/Matrigel. Tumor measurements were taken twice weekly, using vernier calipers. Tumor volume was calculated using the formula: V=0.5·times·width·times·width·times·length.

When tumors approached a volume of 100 mm³, mice were randomized into three groups of ten animals. Mice were treated with vehicle control (10% Solutol® HS15 in saline), compound 3 or 4 at 10 mg/kg, compounds 2, 7, 8, 11, 14 at 20 mg/kg, or 30 mg/kg compound 13; compound 5 was given at 30 mg/kg by intravenous injection. Mice were dosed twice weekly for the duration of the study. Twenty-four hours after the final dose tumor volumes were measured again for calculation of tumor growth inhibition. All statistical analyses were performed using GraphPad PRISM® Version 6.00. Final tumor volumes were analyzed using with a one-way analysis of variance and Tukey multiple comparison test.

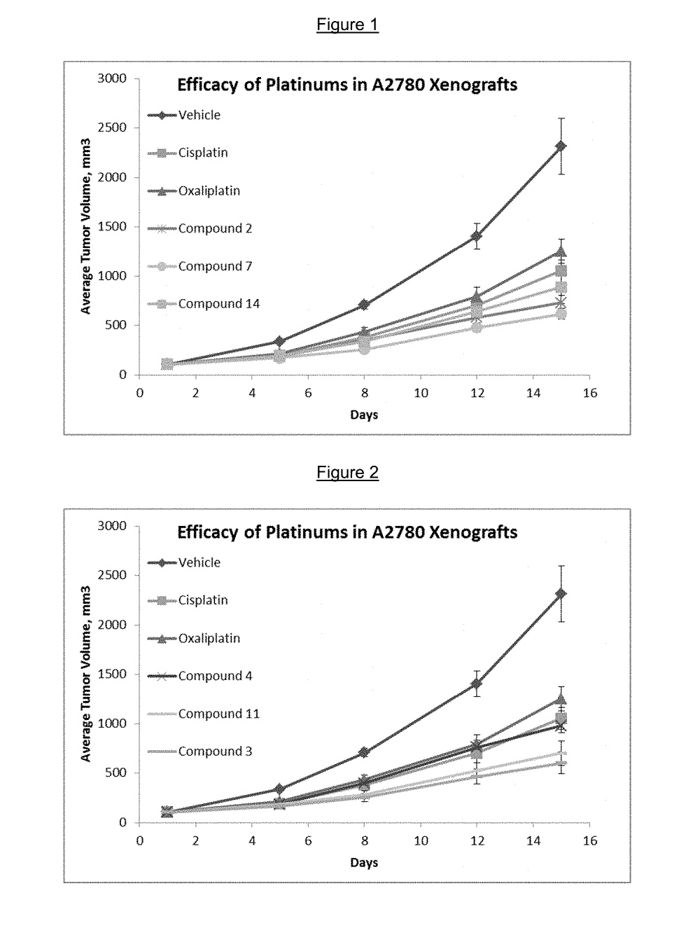

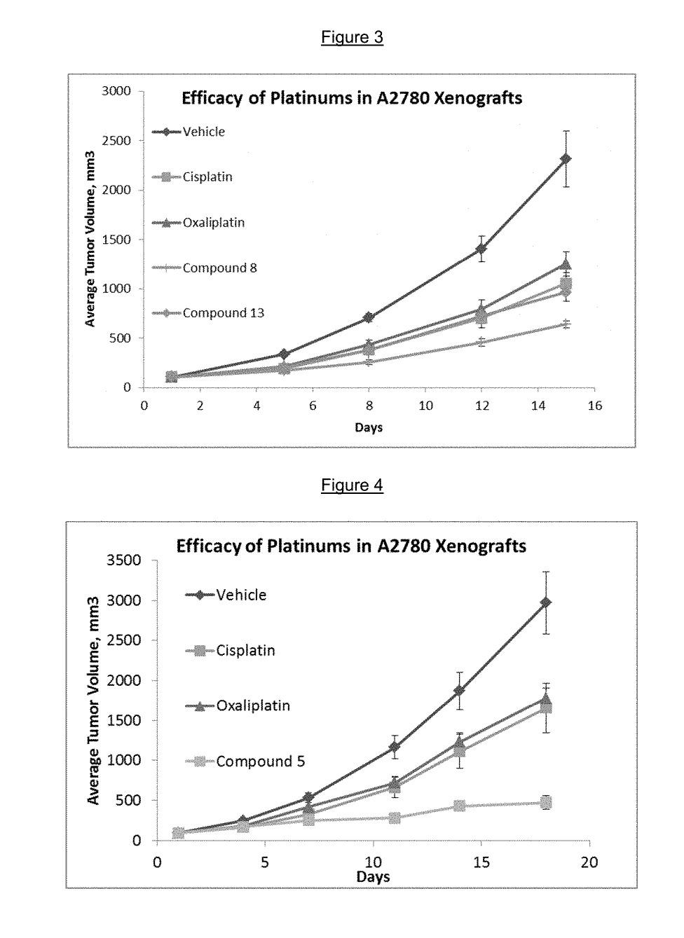

Efficacy data for eight compounds in the A2780 model are shown in FIGS. 1-3. Table 2 shows the percent tumor growth inhibition observed in this study for the eight compounds and cisplatin and oxaliplatin as comparators. All of the tested compounds had increased inhibition of tumor growth compared to cisplatin and oxaliplatin in this model.

TABLE 2

| Treatment | TGI % | pValue |
|---|---|---|
| Cisplatin | 54.5 | p < 0.001 |
| Oxaliplatin | 45.9 | p < 0.001 |
| 4 | 57.7 | p < 0.001 |
| 2 | 68.5 | p < 0.001 |
| 7 | 73.3 | p < 0.001 |
| 8 | 72.4 | p < 0.001 |
| 11 | 69.6 | p < 0.001 |
| 3 | 74.0 | p < 0.001 |
| 13 | 58.3 | p < 0.001 |
| 14 | 61.7 | p < 0.001 |

Efficacy data for compound 5 in the A2780 model are shown in FIG. 4. Table 3 shows the percent tumor growth inhibition (TGI %) observed in this study for compound 5 and cisplatin and oxaliplatin as comparators.

TABLE 3

| Treatment | TGI % | pValue |
|---|---|---|
| Cisplatin | 44.2 | p < 0.01 |
| Oxaliplatin | 40.1 | p < 0.05 |
| 5 | 84.1 | p < 0.001 |

Efficacy data for compound 5 in the Calu-6 model are shown in FIG. 5. Table 4 shows the percent tumor growth inhibition (TGI %) observed in this study for compound 5 and cisplatin and oxaliplatin as comparators.

TABLE 4

| Treatment | TGI % | pValue |
|---|---|---|
| Cisplatin | 91.4 | p < 0.001 |
| Oxaliplatin | 56.7 | p < 0.001 |
| 5 | 90.7 | p < 0.001 |

These data demonstrate that despite the weak inhibition of cell proliferation shown by compounds in vitro, surprisingly, the compounds demonstrated inhibition of tumor growth in xenografts equal to or greater than that of oxaliplatin and cisplatin. It is also of note that while oxaliplatin exhibited significantly different amounts of inhibition in the two models that were tested, compound 5 was highly effective for inhibiting tumor growth in both models.

### Example 22: Tumor Accumulation of Pt(IV)M Monomaleimide Compounds

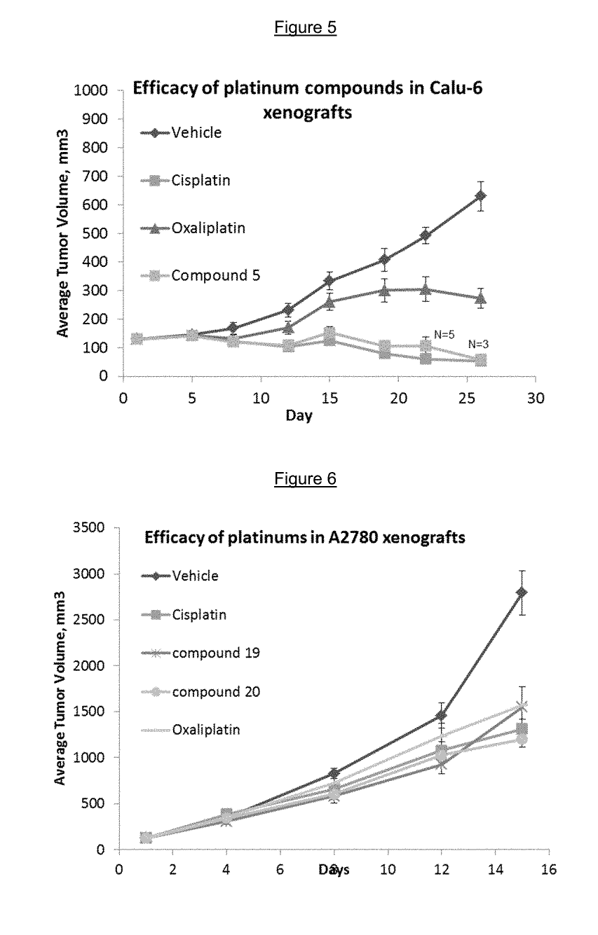

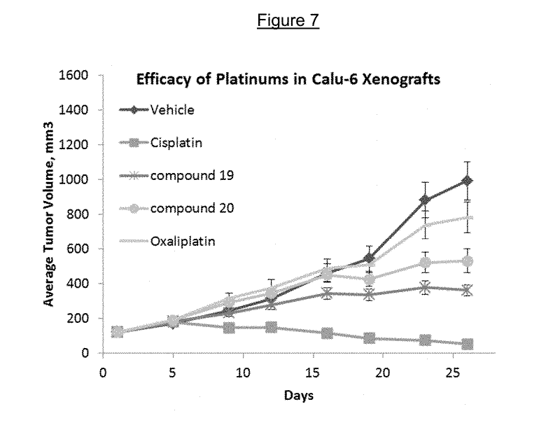

Applicants previously disclosed two platinum-maleimide compounds (see U.S. Ser. No. 61/922,274). The efficacy data for two maleimide compounds previously disclosed that they are similar to cisplatin and oxaliplatin in the A2780 model and intermediate between oxaliplatin and cisplatin in the Calu-6 model (FIGS. 6 and 7) and are inferior in tumor growth inhibition compared to the molecules disclosed in the present application. The tables below shows the percent tumor growth inhibition (TGI %) observed in these studies for compounds 19 and 20 and cisplatin and oxaliplatin as comparators.

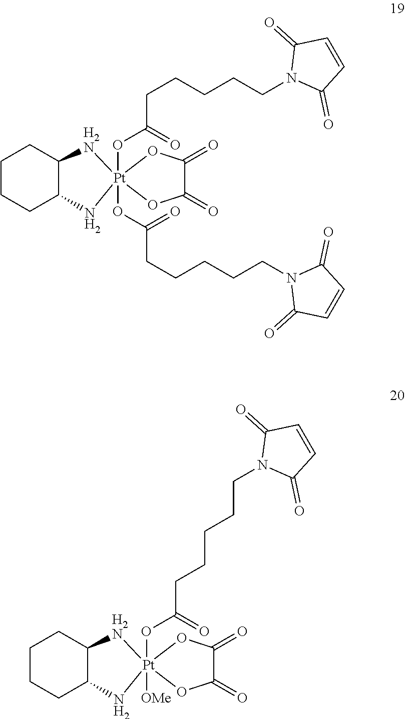

TABLE 5
A2780 xenografts

| Treatment | TGI % | pValue |
|---|---|---|
| Cisplatin | 53.04 | p < 0.001 |
| 19 | 44.53 | p < 0.001 |
| 20 | 56.98 | p < 0.001 |
| Oxaliplatin | 43.75 | p < 0.001 |

TABLE 6
Calu-6 xenografts

| Treatment | TGI % | pValue |
|---|---|---|
| Cisplatin | 94.57 | p < 0.001 |
| 19 | 63.57 | p < 0.001 |
| 20 | 46.47 | p < 0.05 |
| Oxaliplatin | 21.19 | p > 0.05 |

### Example 23: Tumor Platinum Levels in Tumor-Bearing Nude Mice Dosed with Pt(IV)M Compounds

To examine the ability of compounds disclosed herein to accumulate in tumors, a murine cancer model was used. Animals were inoculated with 5×10⁵ H460 small cell lung cancer cells via subcutaneous injection to the flank. Tumors were allowed to reach an approximate volume of ˜500 mm³. Animals were then randomized into treatment groups of 3 animals per time point and were dosed at 4 mg/kg. The 24-hour time point was used as a benchmark across compounds.

Tumor platinum levels were determined by inductively coupled plasma mass spectrometry (ICP-MS). Tumors were excised from animals and dissolved in fuming nitric acid (60% w/w) by adding four parts nitric acid to 1 part tumor w/w and heating overnight at 60° C. The resulting digest was diluted 1:10 in ICP-MS analysis buffer (1% nitric acid, 2% Triton® X-100), and directly introduced into the ICP-MS unit by peristaltic pump. The end dilution factor for the samples as introduced to the ICP-MS was 50×.

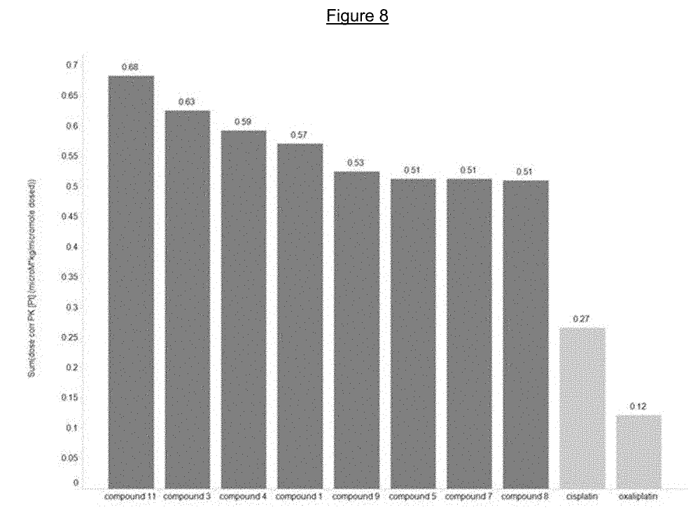

FIG. 8 shows the platinum levels in the tumor for 8 exemplary compounds of the present teachings, respectively, plus cisplatin and oxaliplatin for comparison. These data demonstrate that these compounds show higher platinum levels in the tumors than do cisplatin and oxaliplatin. In addition, these data demonstrate a method of determining the ability of a Pt(IV)M compound to deliver platinum to a tumor.

TABLE 7
Tumor platinum levels in tumor-bearing
nude mice dosed with Pt(IV)M compounds

| | Platinum in tumor |
|---|---|
| | (mM/mmole/kg platinum |
| Compound | dosed) |
| Oxaliplatin | 0.12 |
| Cisplatin | 0.27 |
| 1 | 0.57 |
| 3 | 0.63 |
| 4 | 0.59 |
| 5 | 0.51 |
| 7 | 0.51 |
| 8 | 0.51 |
| 9 | 0.53 |
| 11 | 0.68 |

### Example 24: Reaction of Compound 5 with Albumin or Serum

Without committing to a specific mechanism, it is believed that effective delivery of a Pt(IV)M compound is related to the covalent attachment of the compound to albumin (e.g., human serum albumin; HSA). The circulating amount of albumin in blood is high and the covalent bonding to a Pt(IV)M compound is expected to occur in blood. The compound-albumin bond is cleaved at a tumor site, creating an active platinum compound, e.g., a Pt(II) compound.

To confirm the ability of a Pt(IV)M compound to covalently bind to albumin and that the reaction can occur in blood, an assay system was used in which an HPLC system was coupled to an ICP-MS system to measure the platinum-containing trace for albumin reacted platinum compounds. A 300 angstrom pore size C18 column was run with gradient chromatography to effect separation. The weak buffer was 10 mM pH 5 ammonium formate. The strong buffer was 90% acetonitrile and 10 mM ammonium formate. The gradient was from 0% strong buffer to 100% strong buffer over 9 minutes. The injection volume was 5 uL of sample. Albumin containing samples were directly injected onto the LC-ICPMS system with no dilution, extraction, or other pretreatment.

An authentic sample of compound 5 conjugate to albumin was prepared as follows. A 20% HSA solution was used from Lee Solutions (#R8447). The albumin was diluted to a concentration of 5% (50 mg/mL) with PBS. The conjugation of compound 5 was accomplished as follows: 9 mg of 5 was dissolved in methanol and added to 20 mL of albumin solution (concentration=50 mg/mL). The reaction was conducted for 1 hour at 37 degrees C. After 1 hour, the reaction mixture was washed 25 fold with Water for Injection (WFI) by tangential filtration using a 10 KD membrane cutoff filter (Spectrum Labs). After 500 mL of the washed filtrate was collected, the final albumin:compound 5 conjugate solution was concentrated down to 16 mL. To the concentrate, 1.77 mL of 10×PBS was added to yield an albumin conjugate solution in 1×PBS. The mixture was sterile filtered (0.22 micron Millipore Steriflip). The concentration of albumin in the final solution was determined to be 53.17 mg/mL. The solution was stored at 4 degrees C.

Reactions of compound 5 with serum were conducted by spiking 990 uL of murine serum with 10 uL of compound 5 in DMF. The resulting reaction mixture was a 1:100 dilution of drug with only 1% of solvent present in the biological sample. The reaction was conducted at 37° C. for 30 minutes prior to submission of the sample to the LC-ICPMS analysis queue.

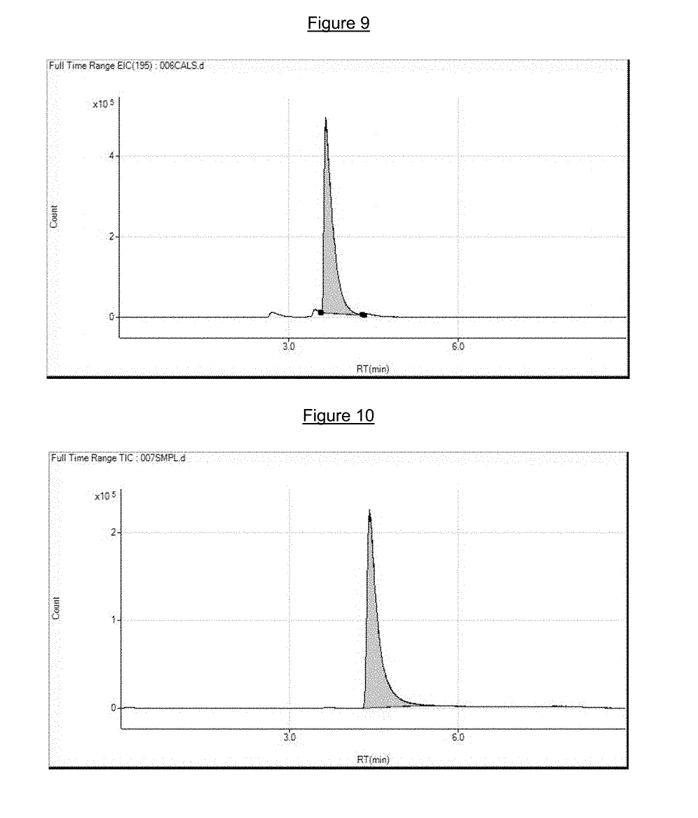

Control samples were reacted to determine the retention times for various platinum containing species. These included compound 5 and compound 5 reacted with albumin. Following the characterization of the chromatography for retention times, rat serum was reacted with compound 5 to determine the extent of reaction with albumin and the specificity of the albumin reaction. Unreacted compound 5 had a retention time of 3.65 minutes (FIG. 9). Commercial human serum albumin reacted with compound 5 had a retention time of 4.35 minutes (FIG. 10).

When compound 5 at a concentration of 300 uM was incubated in murine serum the resulting LC-ICPMS chromatogram showed 96% of the platinum signal was observed to be albumin-bound (FIG. 11).

### Example 25: Uptake of Albumin by KRAS Mutant Cells and Treating KRAS Mutant Tumor Cells with Compound 5

Albumin uptake was measured in BxPC3, NCI-H520, HT-29 wild type KRAS expressing cell lines and in MiaPaCa-2, NCI-H441, HCT-116 and LoVo KRAS mutation expressing cell lines. As used herein, a “KRAS mutant” is a cell, cell line, or tumor that harbors at least one KRAS mutation. FIG. 13 showed that albumin uptake was much higher in KRAS mutant cells compared to cells that do not harbor a KRAS mutation with the exception of LoVo cells. An inhibitor of macropinocytosis decreased albumin uptake.

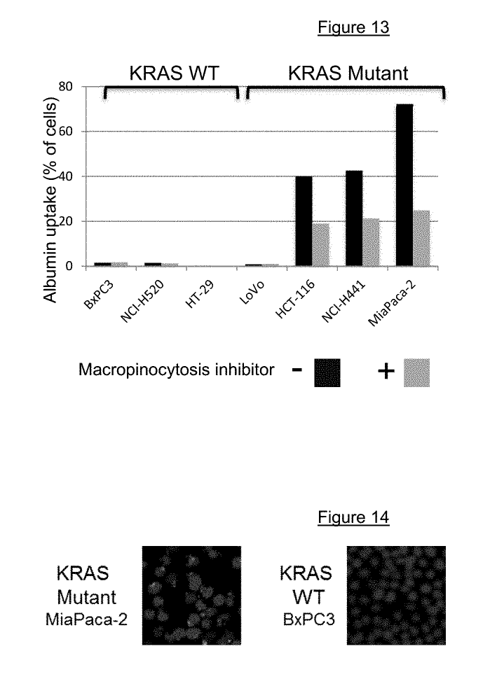

An example of albumin uptake measurements using fluorescently labeled albumin as an imaging agent was shown in FIG. 14. Fluorescently labeled albumin accumulates in KRAS mutant pancreatic Mia PaCa-2 cells more than in KRAS WT pancreatic BxPC3 cells. Radiolabelled albumin may be used as an imaging agent in humans.

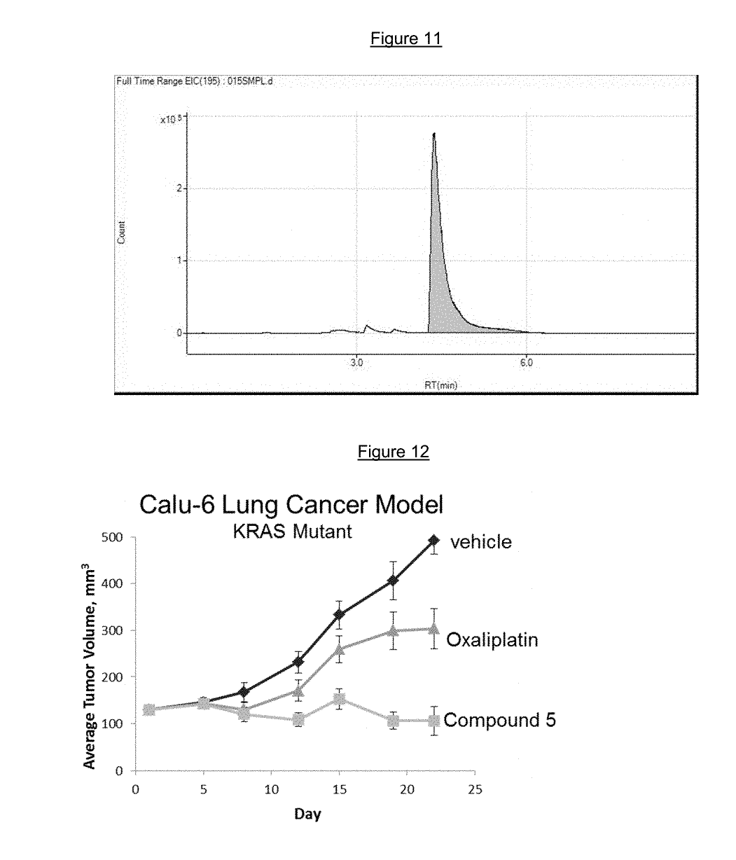

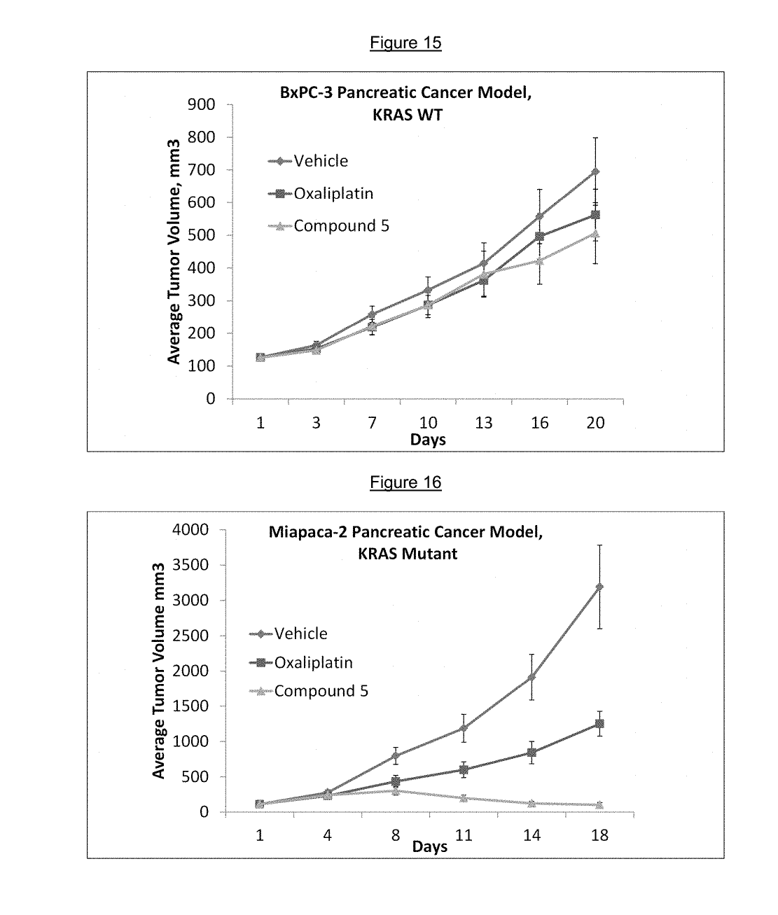

The effect of Pt(IV)M monomaleimide compounds on the growth of KRAS mutant and wild type expressing cells was tested using the same method as described in Examples 21-23. Efficacy data for Compound 5 was shown in FIG. 12, FIG. 15 and FIG. 16. Compound 5 demonstrated inhibition of tumor growth greater than that of oxaliplatin in the KRAS mutant cell lines, Miapaca-2 and Calu-6 as compared to the KRAS wild type line, BxPC-3. Compound 5 was highly effective for inhibiting tumor growth in KRAS mutant models Calu-6 and Miapaca-2.

### Example 26: Pt(IV)M Monomaleimide Compounds Show Increased Tumor Growth Inhibition than Cisplatin and Oxaliplatin

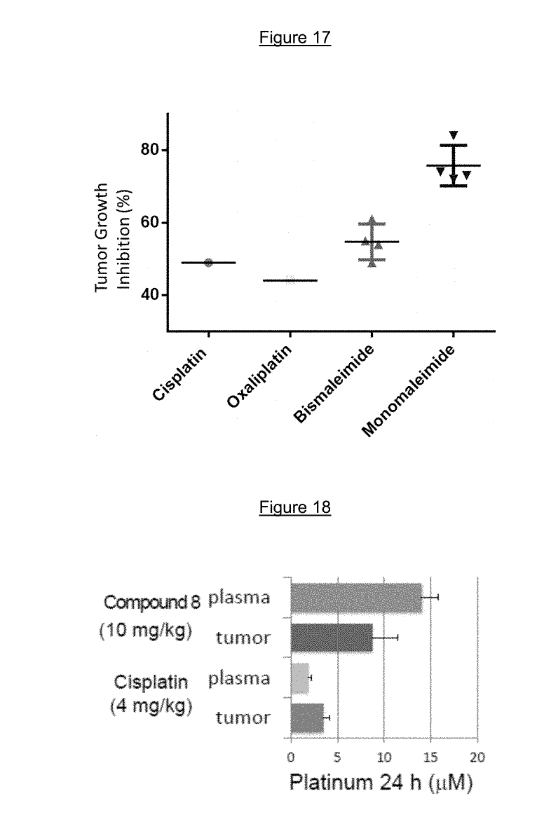

Pt(IV)M monomaleimide compounds (compounds 1, 3, 8 and 11) and compounds comprising two maleimide groups (bismaleimide compounds 19, 28, 29 and 30) were screened in vivo for tumor growth inhibition (TGI %) in A2780 model (ovarian cancer). Cisplatin and oxaliplatin were also tested. Results in FIG. 17 showed that Pt(IV)M monomaleimide have superior TGI % to cisplatin and oxaliplatin.

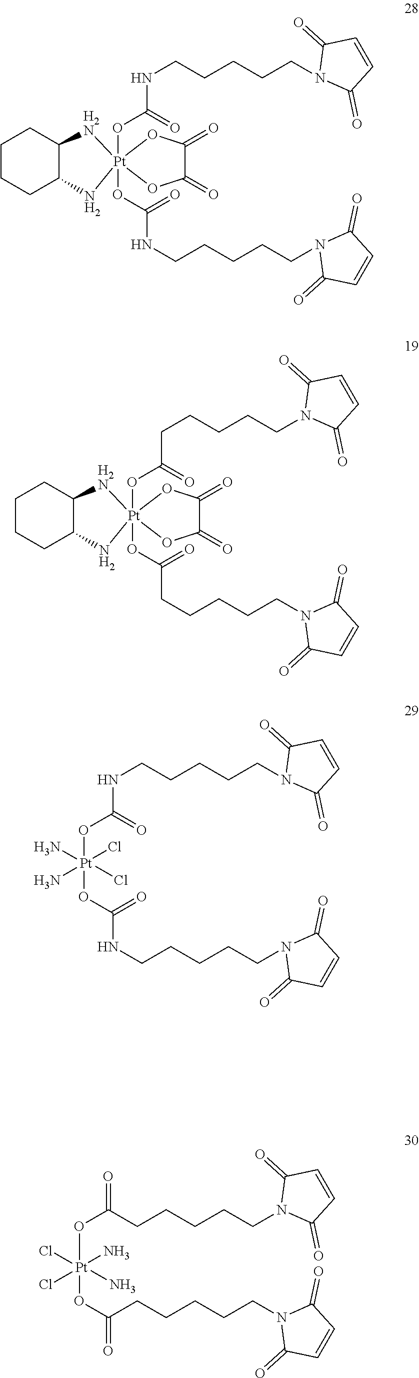

Not willing to be bound to any theory, the second maleimide in bismaleimide compounds has potential to covalently link to other cysteins and lead to cross-linking or toxicity.

### Example 27: Increased Platinum Accumulation and DNA Platination Resulting in Increased Tumor Growth Inhibition than Cisplatin

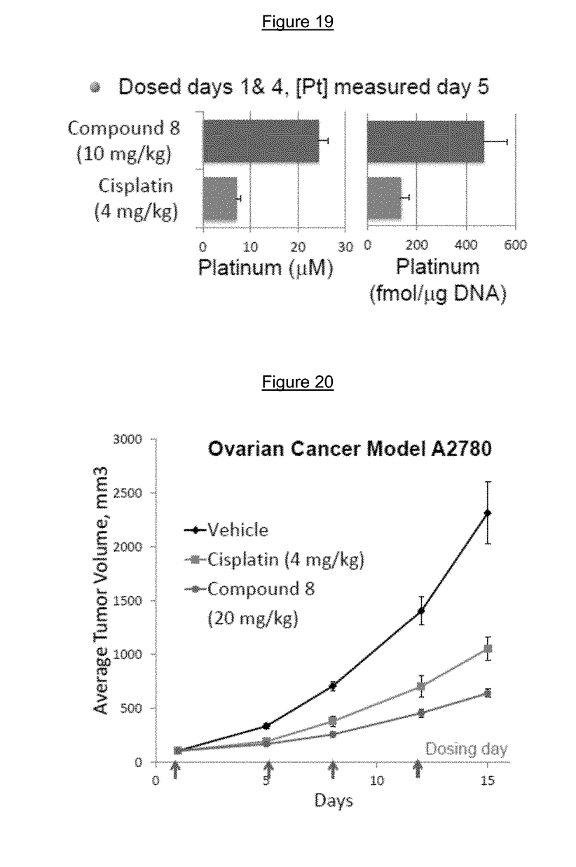

Platinum levels in plasma and tumor 24 hr after a single dose of compound 8 (BTP-114) at 10 mg/kg were measured in lung cancer model NCI-H460. Platinum levels in plasma and tumor at day 5 after two doses of compound 8 (doesed at days 1 and 4) at 10 mg/kg were measured in lung cancer model NCI-H520. Cisplatin was also tested. Results in FIG. 18 and FIG. 19 showed that compound 8 yielded a higher platinum accumulation in both plasma and tumor than cisplatin in both single does and two-dose studies. Increased tumor platinum results in higher levels of DNA platinum adducts.

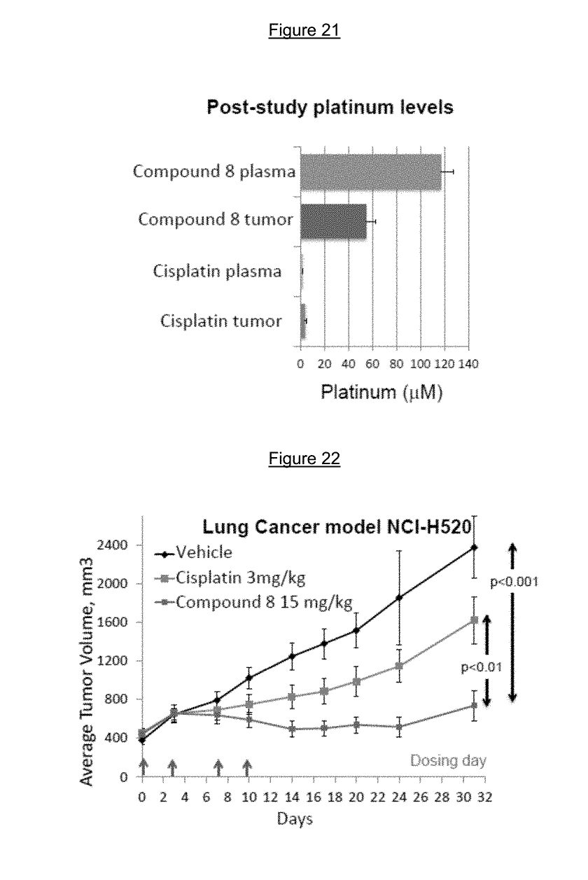

Tumor growth inhibition was measured in ovarian cancer model A2780 for treatment with compound 8 at 20 mg/kg and cisplatin. Results were shown in FIG. 20. Post-study platinum levels were also track and shown in FIG. 21. The concentration of platinum in A2780 xenograph tumor tissue treated with compound 8 was 14 fold higher than cisplatin.

Therefore, Pt(IV)M monomaleimide compound 8 provides higher platinum concentration in tumor tissues than cisplatin and has superior efficacy in tumor inhibition compared to cisplatin.

### Example 28: Pt(IV)M Monomaleimide Compounds Delivers a Sustained Amount of Platinum to Tumors

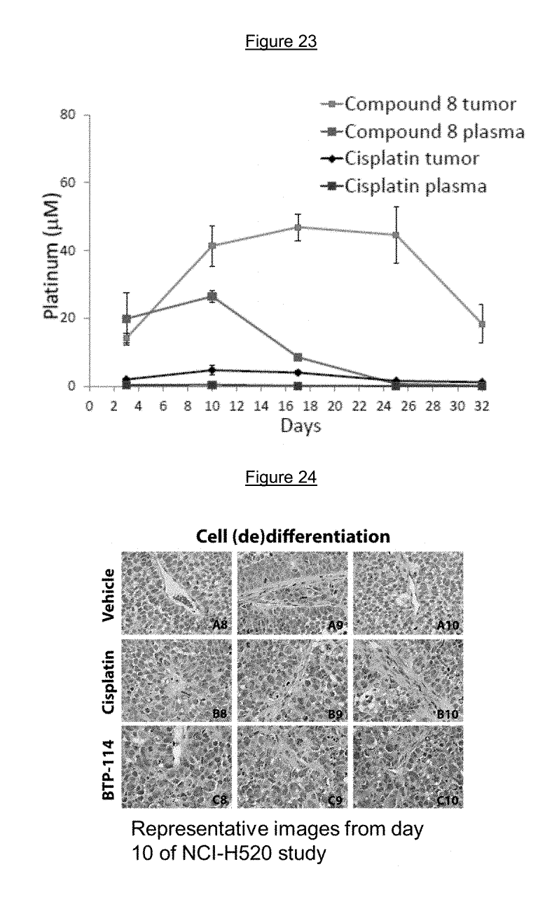

Tumor volumes and platinum levels were tracked over a course of 32 days after treatment of multiple doses of compound 8 (dosed at days 0, 3, 7 and 10) in lung cancer (NSCLC) model NCI-H520. Compound 8 was dosed at 15 mg/kg. Cisplatin was used as a compare and was dosed at 3 mg/kg. The results were shown in FIG. 22 and FIG. 23. Similarly high platinum levels were observed in tumor 16 days after the last dose at day 10. Sustained release of platinum results in superior tumor growth inhibition than cisplatin.

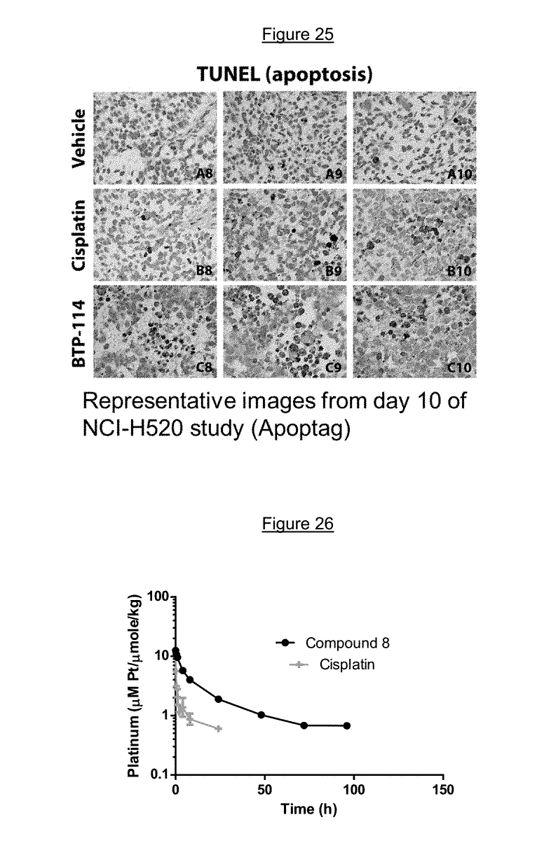

Cell dedifferentiation and apoptosis were studied with immunohistochemical (IHC) and TUNEL stainings. Representative images from day 10 of NCI-H520 model were shown in FIG. 24 and FIG. 25. Tumors treated with compound 8 showed significant and sustained increase in apoptosis and dedifferentiation. These results are consistant with superior efficacy and platinum concentrations of compound 8 compared to cisplatin.

### Example 29: Pharmacokinetics and Distribution of Pt(IV)M Monomaleimide Compounds

Platinum concentrations in rat (n=3) and dog (n=3) were both measured in a course of 100 hours and 400 hours, respectively. Cisplatin was also tested as a compare. Results were shown in FIGS. 26 and 27. Half life of compound 8 in rat and dog was calculated and shown in Table 8 below. Albumin was used as a control.

Platinum exposure of compound 8 is 15-18 fold higher than cisplatin. Half-life of compound 8 paralles albumin. Therefore, Pt(IV)M monomaleimide compounds provide a sustained release of platinum and overcomes the rapid clearance of currently available platinum drug.

TABLE 8
Half-life of compound 8 and albumin in rat and dog

| Half-life | Rat (h) | Dog (h) |
|---|---|---|
| Compound 8 | 44 | 174 |
| Albumin | 55 | 211 |

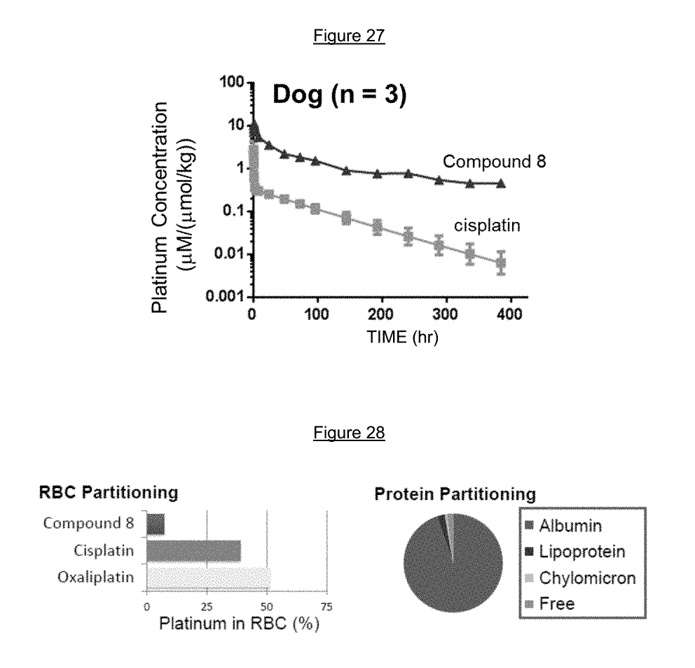

RBC partitioning and protein partitioning were studied. Platinum in RBC and in proteins were measured after treatment with compound 8. Results in FIG. 28 showed that compound 8 is not sequestered in red blood cells and 98% of compound 8 is bound to protein in plasma.

### Example 30: A Brca1/2 Mutant Model is Ultra-Sensitive to Pt(IV)M Monomaleimide Compounds

In vivo MX-1 human breast cancer xenograft studies were carried out with cisplatin and a Pt(IV)M monomaleimide compound. Tumor volumes of MX-1 induced xenografts in mice were measured after multiple intravenous doses of compound 8 at 15 mg/kg at day 1, day 5, day 8 and day 12 (two times per week for two weeks). Compound 8 at 10 mg/kg and cisplatin at 3 mg/kg were also tested with the same dosing days. MX-1 cell line is estrogen receptor negative and Her2 normal. It is generic and BRCA1 and BRCA2 mutant: BRCA1 truncating mutation (3363delGAAA) and BRCA2 mutations (16864A>C, Asn289His, and 22184A>G, Asn991Asp). Average tumor volumes and platinum levels in the tumors were shown in FIG. 29 and Table 9. % T/C in Table 9 is the tumor to control ratio, i.e., the size of tumor divided by the size of tumor control. The lower % T/C is, the higher TGI % is. TGI % at day 15 was shown in Table 10. Tumor disappeared at day 15 after treatment with compound 8. Compound 8 also delivered higher concentrations of platinum to the tumor than cisplatin.

TABLE 9

| | | | | |
|---|---|---|---|---|
| | Mean tumor | | Median tumor | |
| Compound | volume (mm³) | % T/C | volume (mm³) | % T/C |

Tumor volumes at Day 1

| | | | | |
|---|---|---|---|---|
| Vehicle control | 152.7 | | 147.2 | |
| Compound 8 | 153.4 | 100.5% | 150.0 | 101.9% |
| (10 mg/kg) | | | | |
| Compound 8 | 152.0 | 99.6% | 143.7 | 97.6% |
| (15 mg/kg) | | | | |
| Cisplatin (3 mg/kg) | 153.6 | 100.6% | 155.2 | 105.5% |

Tumor volumes at Day 5

| | | | | |
|---|---|---|---|---|
| Vehicle control | 357.2 | | 326.4 | |
| Compound 8 | 308.0 | 86.2% | 284.4 | 87.1% |
| (10 mg/kg) | | | | |
| Compound 8 | 357.1 | 100.0% | 334.1 | 102.3% |
| (15 mg/kg) | | | | |
| Cisplatin (3 mg/kg) | 343.7 | 96.2% | 350.2 | 107.3% |

Tumor volumes at Day 8

| | | | | |
|---|---|---|---|---|
| Vehicle control | 638.8 | | 621.4 | |
| Compound 8 | 272.5 | 42.7% | 275.3 | 44.3% |
| (10 mg/kg) | | | | |
| Compound 8 | 243.3 | 38.1% | 224.6 | 36.1% |
| (15 mg/kg) | | | | |
| Cisplatin (3 mg/kg) | 251.2 | 39.3% | 266.3 | 42.9% |

Tumor volumes at Day 12

| | | | | |
|---|---|---|---|---|
| Vehicle control | 1163.8 | | 1074.8 | |
| Compound 8 | 101.6 | 8.7% | 93.6 | 8.7% |
| (10 mg/kg) | | | | |
| Compound 8 | 81.9 | 7.0% | 80.4 | 7.5% |
| (15 mg/kg) | | | | |
| Cisplatin (3 mg/kg) | 57.6 | 4.9% | 57.6 | 5.4% |

Tumor volumes at Day 15

| | | | | |
|---|---|---|---|---|
| Vehicle control | 1893.1 | | 1942.6 | |
| Compound 8 | 42.9 | 2.3% | 42.2 | 2.2% |
| (10 mg/kg) | | | | |
| Compound 8 | 30.9 | 1.6% | 33.0 | 1.7% |
| (15 mg/kg) | | | | |
| Cisplatin (3 mg/kg) | 26.7 | 1.4% | 29.7 | 1.5% |

TABLE 10
TGI % at Day 15

| | TGI % Day 15 |
|---|---|
| Compound 8 (10 mg/kg) | 97.7 |
| Compound 8 (15 mg/kg) | 98.4 |
| Cisplatin (3 mg/kg) | 98.6 |

### Example 31: Rat Toxicology Studies for Pt(IV)M Monomaleimide Compounds

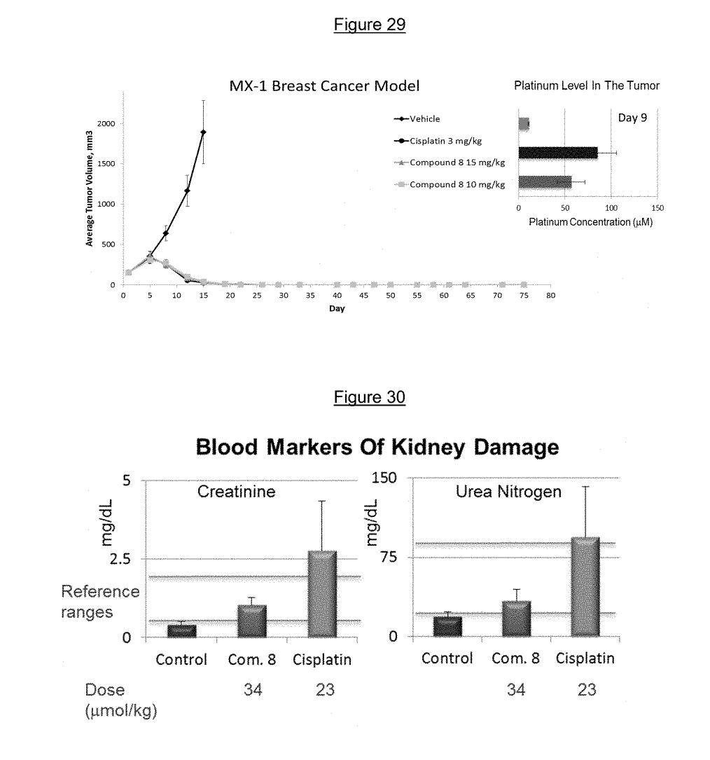

Compound 8 was evaluated in a rat toxicology study. Tubular necrosis values were shown in Table 11. Creatinine and urea nitrogen levels were shown in FIG. 30. Blood markers and histopathology showed an improvement in kidney toxicity compared to cisplatin at a higher dose of platinum. No other histopathology findings of compound 8 were observed.

TABLE 11
Kidney histopathology of cisplatin and compound 8

| | Dose (umol/kg) | Tubular necrosis |
|---|---|---|
| Cisplatin | 23 | 2.3 |
| Compound 8 | 17 | 0 |
| Compound 8 | 34 | 1.3 |

### EQUIVALENTS AND SCOPE

While several embodiments of the present teachings have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present teachings. More generally, those skilled in the art will appreciate that all parameters, dimensions, materials, and configurations described herein are meant to be exemplary and that the actual parameters, dimensions, materials, and/or configurations will depend upon the specific application or applications for which the teachings of the present teachings is/are used. Those skilled in the art will recognize, or be able to ascertain using no more than routine experimentation, many equivalents to the specific embodiments of the present teachings described herein.

It is, therefore, to be understood that the foregoing embodiments are presented by way of example only and that, within the scope of the appended claims and equivalents thereto, the present teachings may be practiced otherwise than as specifically described and claimed. The present teachings are directed to each individual feature and/or method described herein. In addition, any combination of two or more such features and/or methods, if such features and/or methods are not mutually inconsistent, is included within the scope of the present teachings.

The scope of the present invention is not intended to be limited to the above Description, but rather is as set forth in the appended claims.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

1. A composition comprising a Pt(IV)M compound of Formula I: 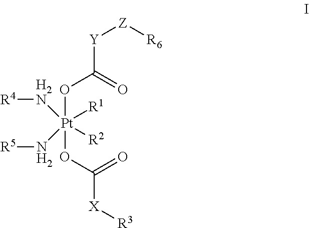
or a pharmaceutically acceptable salt thereof and at least one other active agent, wherein:
X and Y are independently selected from NH, alkyl and aryl;
R¹ and R² each is Cl, or R¹ and R² are joined to form an oxalate;
R³ is hydrogen, alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl-ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl,
R⁴ and R⁵ are each H or together constitute a cyclohexyl ring,
Z is alternatively absent, alkyl, aryl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl-ether, alkoxy, aryloxy, amino, amide, carbamate, alkyl, alkenyl, alkynyl, aryl, arylalkyl, cycloalkyl, heteroaryl, heterocyclyl, or alkylidene hydrazine wherein each of the carboxyl, carbamoyl, ether, alkoxy, aryloxy, amino, amide, carbamate, alkyl, alkenyl, alkynyl, aryl, arylalkyl, cycloalkyl, heteroaryl, heterocyclyl or alkylidene hydrazine is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl,
R⁶ is a suitable reacting group for reacting with a functional group on a protein, engineered protein, antibody, antibody fragment, peptide, agonist, antagonist, aptamer or any moiety which may be capable of recognizing a selected target cell population, and/or derivatives/analogs/mimics thereof, selected from an activated disulfide group, a vinylcarbonyl group, a vinyl acetylene group, an aziridine group, an acetylene group or any of the following groups: 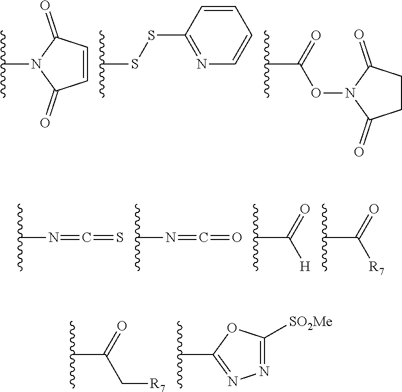
where R⁷ is C1, Br, F, mesylate, tosylate, O-(4-nitrophenyl), O-pentafluorophenyl, and wherein optionally the activated disulfide group, the vinylcarbonyl group, the vinyl acetylene group, the aziridine group, the acetylene group may be substituted.

2. The composition of claim 1, wherein the Pt(IV)M compound and the at least one other active agent may be present as a mixture or may each be present in separate containers.

3. The composition of claim 1, wherein the protein is albumin.

4. The composition of claim 1, wherein R6 is a maleimide group and the compound has Formula II:

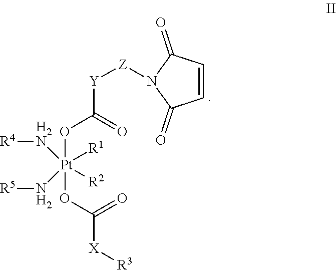

5. The composition of claim 4, wherein the compound has Formula IIa:

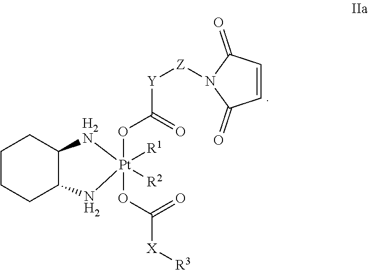

6. The composition of claim 4, wherein the compound has Formula IIb:

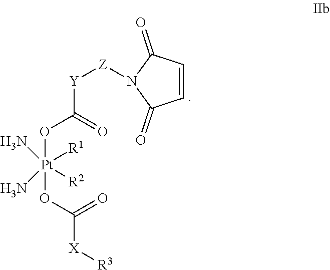

7. The composition of claim 1, wherein R¹ and R² each is C1.

8. The composition of claim 1, wherein R¹ and R² are joined to form an oxalate.

9. The composition of claim 1, wherein R³ is alkyl.

10. The composition of claim 1, wherein R³ is methyl or ethyl.

11. The composition of claim 1, wherein the compound is selected from:

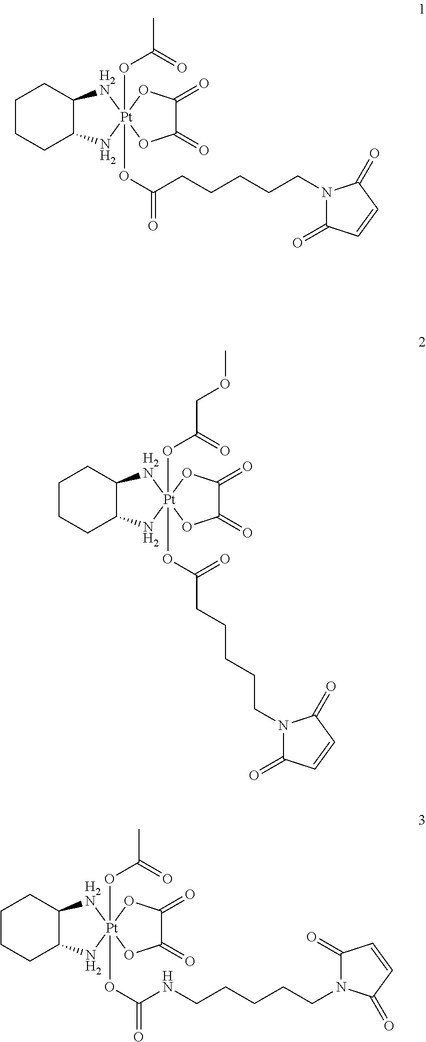

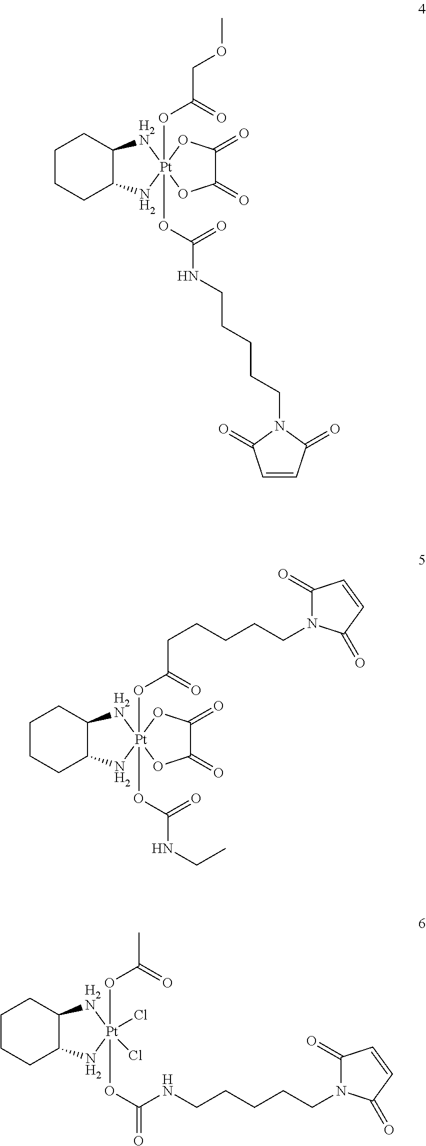

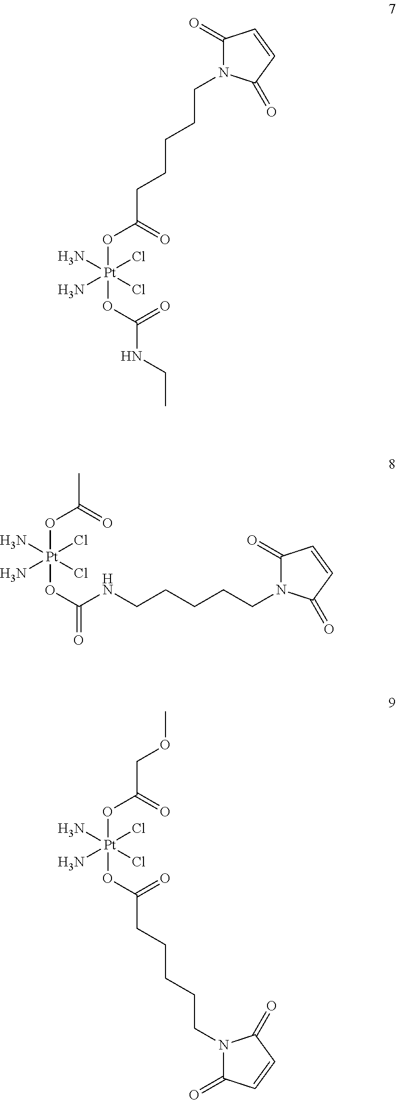

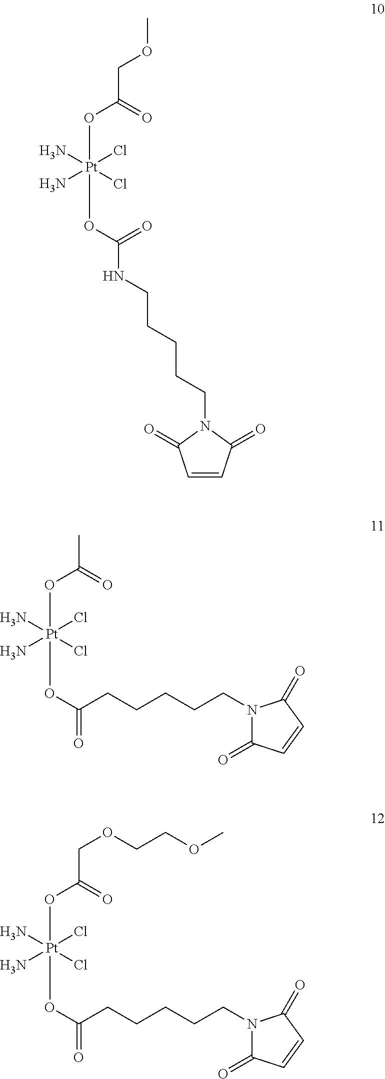

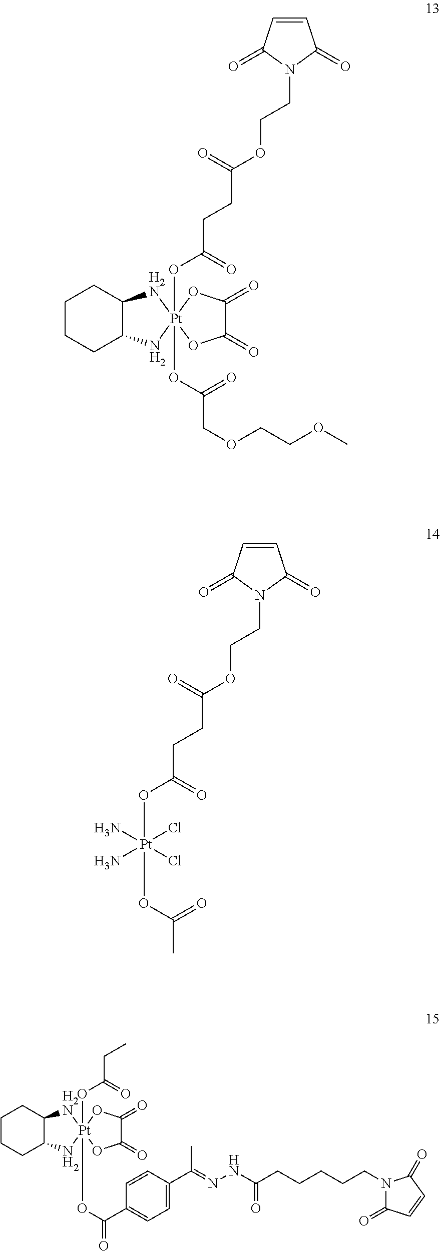

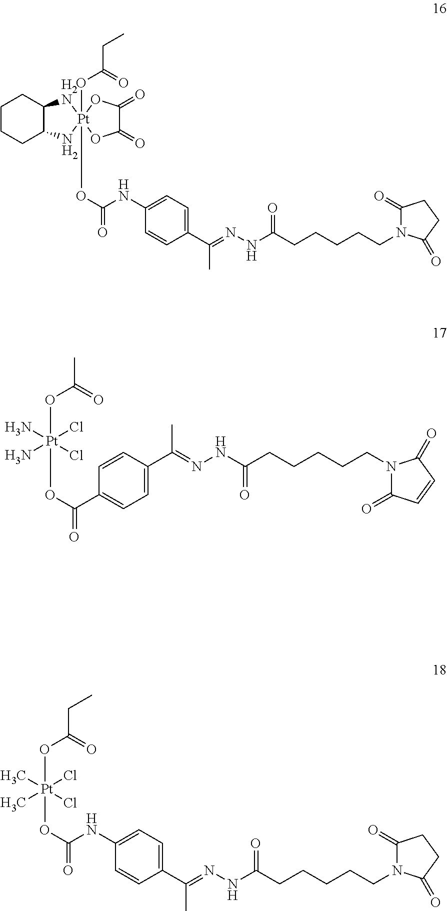

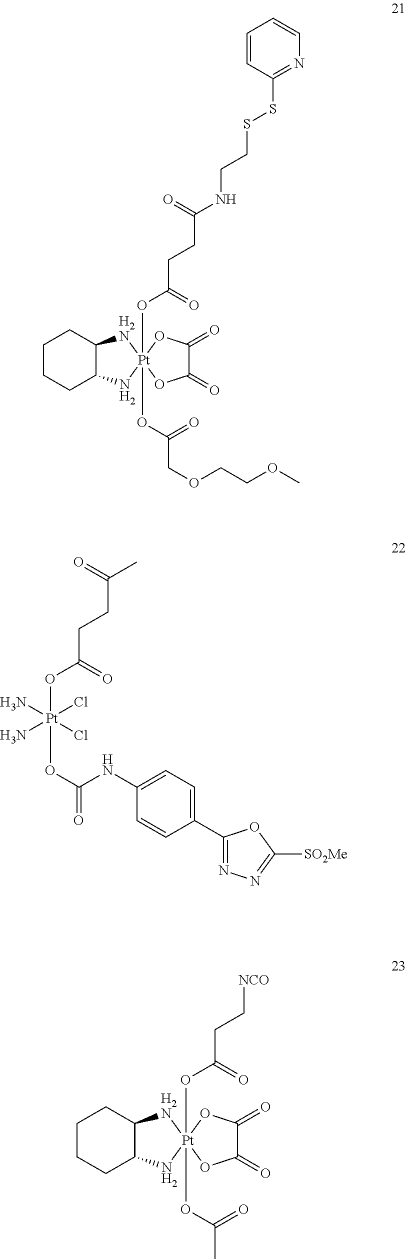

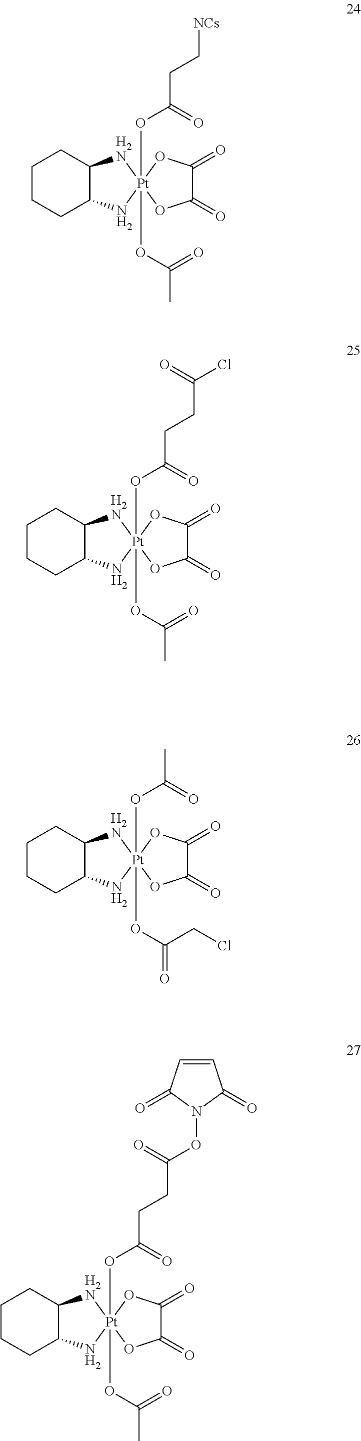

12. The composition of claim 1, wherein the at least one other active agent may be a therapeutic, prophylactic, diagnostic, or nutritional agent.

13. The composition of claim 12, wherein the at least one other active agent is selected from a small molecule, protein, peptide, lipid, glycolipid, glycoprotein, lipoprotein, carbohydrate, sugar, or nucleic acid.

14. The composition of claim 1 further comprising a pharmaceutically acceptable carrier.

15. A method of treating a disease comprising administering a therapeutically effective amount of the composition of claim 1, wherein the disease is selected from cancer and an inflammatory disease.

16. The method of claim 15, wherein the disease is cancer.

17. The method of claim 16, wherein the cancer is selected from lung cancer including small cell lung cancer, non-small cell lung cancer and squamous cell lung cancer, breast cancer, colorectal cancer, colon cancer, ovarian cancer, pancreatic cancer-bladder cancer, prostate cancer, cervical cancer, renal cancer, leukemia, central nervous system cancers, myeloma, melanoma, mesothelioma, stomach cancer, rectal cancer, cancer of the large intestine, cancer of the small intestine, esophageal cancer, uterine cancer, head and neck cancer, endometrial cancer, eye cancer, thyroid cancer, testicular cancer, bile duct cancer, liver cancer, kidney cancer, pituitary cancer, lymphoma, brain cancer, glioma, glioblastoma multiforme, meningioma, medulloblastoma, astrocytoma, neuroblastoma, basal cell carcinoma of the skin, sarcoma, synovial sarcoma, rhabdomyosarcoma, leiomyosarcoma, chondrosarcoma, and fibrosarcoma.

18. The method of claim 15, further comprising conducting a chemotherapy, a surgical treatment, a radiation therapy, an immunotherapy, and/or with any other antineoplastic treatment method.

19. A method of inhibiting proliferation of a cell comprising contacting the cell with an effective amount of the composition of claim 1.

20. The method of claim 19, wherein the cell is a cancer cell.

21. A method of inhibiting the rate of growth of a tumor, the size of a tumor or the volume of a tumor the method comprising contacting the tumor with an effective amount of the composition of claim 1.

22. A method of delivering platinum to a tumor in a subject, the method comprising administering a composition of claim 1 to the subject.

23. A method of treating cancer in a subject, the method comprising:
a. identifying a subject having or at the risk of developing cancer, wherein the subject has been identified as having a KRAS mutation; and
b. treating the subject with the composition of claim 1.

24. A method of reducing tumor volume in a subject, the method comprising:
a. determining if the subject has a KRAS mutation; and
b. treating the subject with the composition of claim 1 if the subject has a KRAS mutation.

25. The method of claim 24, wherein the subject has a tumor comprising cells that express at least one KRAS mutant.

26. The method of claim 24, wherein the tumor of the subject is assayed for the presence of a KRAS mutation.

27. The method of claim 24, wherein a non-tumor tissue is assayed for the presence of a KRAS mutation.

28. The method of claim 27, wherein the non-tumor tissue is plasma DNA.

29. The method of claim 25, wherein the KRAS mutation is homozygous.

30. A Pt(IV)M compound comprising 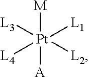
or a pharmaceutically acceptable salt thereof, wherein M is a ligand comprising any suitable group for reacting with a function group on a protein, engineered protein, antibody, antibody fragment, peptide, agonist, antagonist, aptamer or any moiety which may be capable of recognizing a selected target cell population, and/or derivatives/analogs/mimics thereof; A is a ligand comprising at least one active agent; and L₁, L₂, L₃, and L₄ may be any suitable ligand for Pt.

31. The Pt(IV)M compound of claim 30, wherein the protein is albumin and M is a ligand comprising an albumin-binding group.

32. The Pt(IV)M compound of claim 31, wherein M comprises a maleimide group or a derivative/analog thereof.

33. The Pt(IV)M compound of claim 32, wherein the compound has a Formula A of 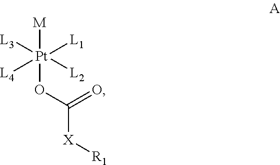
wherein X is selected from NH, alkyl and aryl;
R1 comprises an active agent;
L₁, L₂, L₃ and L₄ are independently any suitable ligand for Pt;
M comprises a suitable reacting group for reacting with a functional group on a protein, engineered protein, antibody, antibody fragment, peptide, agonist, antagonist, aptamer or any moiety which may be capable of recognizing a selected target cell population, and/or derivatives/analogs/mimics thereof, such as but not limited to: 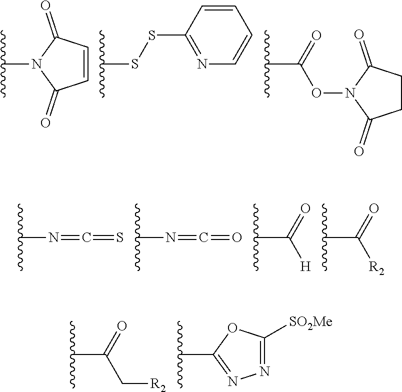
where R2 is Cl, Br, F, mesylate, tosylate, O-(4-nitrophenyl), O-pentafluorophenyl; an activated disulfide group; a vinylcarbonyl group; a vinyl acetylene group; an epoxide; an aziridine group or an acetylene group; wherein the groups may be substituted or not.

34. The Pt(IV)M compound of claim 30, wherein the least one active agent is a therapeutic, prophylactic, diagnostic, or nutritional agent.

35. The Pt(IV)M of claim 30, wherein the at least one other active agent is selected from a small molecule, protein, peptide, lipid, glycolipid, glycoprotein, lipoprotein, carbohydrate, sugar, or nucleic acid.
